(19) United States
(12) Patent Application Publication
(10) Pub. No.: US 2006/0121489 A1
(43) Pub. Date: Jun. 8, 2006

(54) High throughput screening of aptamer libraries for specific binding to proteins on viruses and other pathogens

(75) Inventors: David G. Gorenstein, Houston, TX (US); Bruce A. Luxon, Galveston, TX (US); Allan Barrett, Galveston, TX (US); Michael Holbrook, Oklahoma City, OK (US); Suzanne Bassett, Galveston, TX (US); Anoma Somasunderam, Galveston, TX (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 10/851,947

(22) Filed: May 20, 2004

Related U.S. Application Data
(60) Provisional application No. 60/472,897, filed on May 23, 2003.

(51) Int. Cl. C12Q 1/68

(52) U.S. Cl. 435/6

(57) ABSTRACT

The present invention includes composition and methods for making and using a combinatorial library to identify thioaptamers that bind to targets on or about pathogens. Compositions, kits and methods are also provided for the identification of pathogens, e.g., viral, bacterial or other proteins related infectious disease, as well as, vaccines and vaccine adjuvants are provided that modify host immune responses.

### TECHNICAL FIELD OF THE INVENTION

The present invention relates in general to the field of aptamer libraries, and more particularly, to enhancing availability and use of aptamers for screening, including high-throughput screening and kits, of target molecules on pathogens and cells.

### BACKGROUND OF THE INVENTION

This application claims priority to U.S. Provisional Patent Application Ser. No. 60/472,897, filed May 23, 2003. This work was supported by the following United States Government grants DARPA (9624-107 FP), NIH (A127744) and NIEHS (ES06676). Without limiting the scope of the invention, its background is described in connection with oligonucleotide agents and with methods for the isolation and generation thereof.

Virtually all organisms have nuclease enzymes that degrade rapidly foreign DNA as an important in vivo defense mechanism. The use, therefore, of normal oligonucleotides as diagnostic or therapeutic agents in the presence of most bodily fluids or tissue samples is generally precluded. It has been shown, however, that phosphoromonothioate or phosphorodithioate modifications of the DNA backbone in oligonucleotides can impart both nuclease resistance and enhance the affinity for target molecules.

Recent world events have heightened the awareness of possible bioterrorist threats. Hemorrhagic fever viruses (category A bioweapon agents) have reportedly been weaponized by the former Soviet Union and the United States (Borio et al., 2002; Hawley & Eitzen, 2001). Despite the awareness of the potential of Viral Hemorrhagic Fever viruses (Lassa, Junin), Encephalitic viruses (West Nile, VEE) and other agents both as bioweapons and as emerging viral diseases, few therapeutic options are available to those infected. Apart from supportive therapy, the only drug for treating Arenavirus infections is Ribavirin and it is only partially effective (McCormick et al, 1986a; Shulman, 1984; Enria et al., 1987) while there are no efficacious drugs to treat victims of West Nile infections (Peterson and Marfin, 2002). There is an urgent need to expand the current therapeutic armamentarium, which is hindered, at least in part, by a lack of in-depth knowledge concerning the mechanisms of Arenaviral pathogenesis (Peters & Zaki, 2002).

Arenavirus pathogenesis stems from host immune response dysregulation and endothelial dysfunction (Peters & Zaki, 2002; Ignatyev et al., 2000; McCormick & Fisber-Hoch, 2002; Walker et al., 1982; McCormick et al., 1986b; Marta et al., 1999). West Nile pathogenesis is associated with the inability of host immune response to limit virus replication to levels below that required for viral invasion of the CNS (Solomon and Vaughn, 2002).

Lassa fever, a human arenavirus hemorrhagic fever virus endemic in West Africa, affects up to 300,000 people annually and is responsible for up to 3000 deaths (McCormick, et al., 1987). Lassa Fever virus is difficult to study due to its hazardous nature (a BSL4 agent). Junin Virus is the causative agent of Argentine hemorrhagic fever (AHF). The annual incidence varies between 100-4000 cases/yr. AHF has a case fatality rate of 15-30% and is also a BSL4 agent. A well-established animal model that resembles Lassa Fever, using the non-pathogenic New World Arenavirus, Pichinde virus (Jahrling et al., 1981) has been used to study this class of pathogens. Serial passage of Pichinde virus in guinea pigs was used to develop a virulent variant that produces a disease in guinea pigs that mimics human Lassa Fever in many important respects including: viremia correlates with disease outcome (Johnson et al., 1987; Aronson et al., 1994), a relative paucity of pathologic findings in lethally infected animals (Walker, et al., 1982; Connolly, et al., 1993), terminal vascular leak syndrome (Katz & Starr, 1990) and distribution of viral antigens within the host (Connolly et al., 1993; Shieh et al., 1997; Aronson, unpublished data). Macrophage responses to the attenuated Pichinde virus, P2, with the virulent Pichinde variant, P18 as well as reassortants of the two variants (Zhang et al., 1999; Zhang et al., 2001; Fennewald et al., 2002) may be used to compare and modify the immune response to viral infection.

West Nile virus (Category B virus) is a mosquito-borne flavivirus that is a neuropathogen in humans, equines and avians (Solomon and Vaughn, 2002; Petersen and Marfin, 2002). Humans become infected by the bite of an infected mosquito. The viruses are then thought to replicate in the skin before being transported to the local lymph nodes. West Nile may then spread via the blood to other organs including the liver, spleen, heart and kidney and eventually the brain. West Nile virus may spread to the CNS via either hematogenous spread or via the olfactory mucosa where there is no blood-brain barrier. West Nile is an emerging pathogen in the US, spreading across the country since it was first identified in New York in 1999. As of Oc. 3, 2002, the CDC has reported 2530 cases of West Nile virus infection with 125 deaths in 32 states. West Nile is also responsible for major outbreaks in other countries including Tunisia, Romania, Algeria, Russia and Israel among others. Case fatality rates range from 4-29%. Age is a risk factor in the development of severe West Nile disease with many patients exhibiting substantial morbidity. Presently, treatment for West Nile is limited to supportive intervention. There is no evidence that either interferon or Ribavirin treatment is efficacious (Petersen and Marfin, 2002).

Arenavirus Hemorrhagic Fevers, such as Lassa fever, Junin, Argentine hemorrhagic fever, Bolivian hemorrhagic fever and Venezuelan hemorrhagic fever, have several features in common with sepsis and the systemic inflammatory response syndrome, including fulminant clinical course, fever, shock, capillary leak syndrome, decreased myocardial contractility, abnormalities of coagulation and platelet function, and elevated serum levels of TNFα (Aronson et al., 1994; Cummins, 1990). Arenaviruses are non-cytopathic viruses with a tropism for macrophages and other reticuloendothelial cells (Cummins, 1990; Peters et al., 1987); the pathogenesis of these diseases is believed to involve excessive production of pro-inflammatory cytokines (Aronson et al., 1995; Peters et al., 1987). Unpublished data (Bausch et al., CDC) show cytokines to be massively activated in human Lassa fever, and also confirm that Lassa virus can directly induce cytokine secretion by infecting human macrophages in vitro (Mahanty et al., CDC, unpublished). Alternatively, there is evidence that a swift elaboration of pro-inflammatory cytokines and early engagement of the (innate) immune response may help protect of the infected host from lethal disease in various hemorrhagic fever syndromes (Peters et al., 1987).

One example of art in this area is published U.S. patent application Serial No. 20020061542, filed by David L. Rimm, et al., entitled, “Method For The Detection, Identification, Enumeration And Confirmation Of Virally Infected Cells And Other Epitopically Defined Cells In Whole Blood,” which is a continuation-in-part of U.S. Pat. No. 6,197,523, which describes a method for detection of epitopically-defined cancer cells in whole blood. The patent application describes a method for analyzing blood for the presence of target cells such as lymphocytes, monocytes, granulocytes, nucleated blood cells or the like that have already been infected by a virus. These virall-infected cells must already express surface epitopes that indicate intracellular infection by viruses (e.g. HIV, CMV, HCV) or other infectious agents that are known to be absent in normal blood cells. Therefore, the method determines the presence of a virally-infected blood cell, and not the presence of a free virus and is confined to infected-blood analysis.

### SUMMARY OF THE INVENTION

The present invention demonstrates the use of “thioaptamers™” that are specific for target proteins or other molecules of pathogens. For example, the thioaptamers of the present invention may be used to detect, isolate, concentrate, characterize or even prevent disease by, e.g., a viral, bacterial or other pathogen. The present invention also includes kits and methods for screening pathogens and even transformed cells. Furthermore, the present invention provides for novel therapeutic interventions for the treatment of, e.g, hemorrhagic fevers, encephalitic viruses and other viral infections, resulting from their use as bioweapons or as emerging diseases. For example, modified thioaptamers were used to identify and characterize biopathogens. The thioaptamers may also be isolated, selected, improved and characterized to protect against pathogenic infection. The thioaptamers of the invention may be selected to, e.g., inhibit the binding sites of pathogens for cellular entry, colonization and/or division and release from the host.

The present invention is based on the recognition that thiomodified aptamers may be designed, isolated and used to manipulate, e.g., transcription factors such as NFκB and AP-1 to interdict the pathogenetic sequence, or even boost early protective innate immune responses. The present invention extends these observations and the methods and systems disclosed herein to the area of infectious disease and cancer. To demonstrate the feasibility of using the modified thioaptamers disclosed herein at physiological concentrations, animal model systems were used that models both severe fatal disease and self-limited infection with mild disease. The present invention also uses the modified thio-aptamers to bind to target proteins of pathogens. Using the thioaptamers of the present invention is may be possible to modify responses to diseases involving pathogenic or disfunctional inflammatory responses such as cancer, heart disease, inflammatory bowel disease, rheumatoid arthritis and lupus.

Therefore, in one embodiment, the present invention is a method for isolating pathogen-specific aptamers, which includes the steps of: contacting a pathogen sample that includes a target with an aptamer library and determining the sequence of the one or more aptamers that bind specifically to the target in the pathogen sample. The aptamer library may be immobilized on a substrate, e.g., a bead, a semiconductor, a metal, a film, a chip, a filter and/or glass, and the like. The one or more aptamers in the library may be thio-modified and in some cases may be partially thio-modified aptamers. The library of aptamers may be immobilized on one or more beads that may be, e.g., a one-bead, one-aptamer library or even a library of libraries. The sample may be, e.g., whole cell lysates, concentrates, supernatants or mixtures thereof. The samples may include, e.g., viruses, prokaryotes and eukaryotes or portions thereof. One example of a sample may be a sample suspected of including a bioterror virus, bacteria or toxin.

The method of the present invention may be used to bind, isolate, identify, characterize or otherwise evaluate or concentrate one or more surface molecules of a pathogen, which may be, e.g., a cell or viral surface protein, a lipid, a carbohydrate, an envelope protein or an envelope glycoprotein. The types or sources of samples may be, e.g., an environmental, biologic, aqueous, organic or even an air sample. One specific type of sample may be one or more environmental samples that are being evaluated, e.g., for the presence or absence of a bioterror agent, such as a virus, bacteria, toxin or fractions thereof, e.g., a toxin or active subunits thereof. Identification of the pathogen may be by, e.g., performing a surface enhanced laser desorption ionization mass spectroscopy analysis of the protein from the sample that bound the aptamer, which may be a SELDI/MALDI-TOF analysis of the protein. Alternatively, the method may also include the step of isolating the one or more beads that bind specifically to the pathogen using flow cytometry.

Examples of bioterror agents that may be evaluated include pathogen samples that includes a bioterror agent, e.g., Bacillus, Yersinia, Francisella, Vibrio, Brucella, Clostridium. In another example, the bioterror agent may be, e.g., a flavivirus, a hepadnavirus, a coronavirus, a hantavirus, a smallpox viruse, a hemorrhagic fever virus, and/or a neuropathologic virus. Toxins or their subunits for determination may be Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins, or mixtures thereof. In one particular embodiment, the aptamers and thioaptamers of the present invention may be specific to a Biosafety Level 4 (BSL 4) pathogen.

One particular embodiment of the present invention includes a system adapted for high-throughput screening (HTS) by automating the step of contacting one or more samples to one or more aptamers. For example, the HTS system may be adapted for remote screening of one or more pathogens, in particular when the unknown sample is suspected of being including BSL 3 or BSL 4 pathogens. The remote sensing system may include one or more transceivers that communicate with a user that is located remotely and may include communications via a radio frequency, microwave, ultrasound, infrared or other communication system using equipment and protocols well known to the artisan skilled in network and/or satellite telecommunications.

For use with the present invention the thioaptamers and the thioaptamer library may include in each oligonucleotide (ODN) on or more S—ODN, S₂—ODN, or mixtures thereof. The ODN and the substrate may be attached via a linker molecule. An example of a linker for use with the present invention to attached one or more aptamers to a bead may be, e.g., a hexaethyleneglycol linker, although the skilled artisan will know of may other chemistries that may be used to link biological molecules, such as ODNs to substrates. In some cases the substrate will generally direct the user to the best type of linker. The linker may be added at the beginning of the attachment process (e.g., before a nascent chain of nucleotides or even one or more completed or partially completed oligonucleotides is added) of thereafter, that is, when the ODNs are synthesized and then added to the bead. While any number of targets may bind the aptamers and thioaptamers of the present invention, in one particular embodiment, the aptamers may include one or more motifs associated with, e.g., nuclear transcription factor protein binding site sequences.

When using thioaptamers, these may be, e.g., isosteric, isopolar and/or achiral, have the same length and/or even include one or more detectable markers, e.g., an enzyme, an antibody, a linker, a radioisotope, an electron dense particle, a magnetic particle or a chromophore. For example, when using any one of these detactable labels these may be attached to the 5′-end and/or 3′-end, one or both strands, or even internally within the aptamer or thioaptamer. Alternatively, it may a component of the sample that contains the detectable marker that is detectable after binding to the aptamer or thioaptamer or both the sample and the aptamer or thioaptamer may be labeled. Alternatively, a detectable marker may be an inherent property of the molecule that binds to the aptamer that is detected, e.g., absorption at certain wavelengths, binding to certain chemicals or involvement in a reaction as a co-factor, a reactant or a catalyst. Aptamers and thioaptamers of the present invention may be DNA, RNA or PNAs, may be single or double-stranded further comprise the complementary strand to the aptamer and/or may be modified in one or both strands. The thioaptamers may include both modified and unmodified nucleotides, e.g., dATP(αS), dTTP(αS), dCTP(αS), dGTP(αS), dATP(αS2), dTTP(αS2), dCTP(αS2) and dGTP(αS2).

The method of the present invention may further include the step of amplifying an aptamer library enzymatically using a mix of four nucleotides, wherein at least a portion of at least one and no more than three of the nucleotides in the mix is thiophosphate-modified, to form a partially thiophosphate-modified oligonucleotide combinatorial library. In one example, no more than three adjacent phosphate sites of an individual aptamer in the aptamer library are replaced with phosphorothioate groups. The aptamer library may be created by a split and pool combinatorial synthesis chemistry. In one embodiment, the aptamers of the present invention are isolated nucleic acids having SEQ ID NOS. 30 through 107, and combinations thereof. Another embodiment of the present invention is an aptamer library for use in the method.

In yet another embodiment, the present invention is a pathogen-specific aptamer that is partially thio-modified and that specifically binds to a portion of a pathogen. These pathogen-specific aptamer may be selected from SEQ ID NOS.: 30 to 107. The aptamers may be bind to pathogens that are bioterror agents, e.g., bacteria such as Bacillus, Yersinia, Francisella, Vibrio, Brucella, Clostridium; viruses such as a flavivirus, a hepadnavirus, a coronavirus, hantavirus, smallpox viruses, a hemorrhagic fever virus, and a neuropathologic virus; or toxins such as Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins, mixtures thereof or active subunits thereof. The aptamers and/or thioaptamers may bind specifically to a pathogen that is Biosafety Level 4, e.g, a Hepatitis C virus, Yellow Fever virus, Dengue Virus, Japanese Encephalitis virus, West Nile virus, St. Louis encephalitis virus, Murray Valley encephalitis, tick-borne encephalitis, Omsk hemorrhagic fever virus and Langat virus.

Yet another method of the present invention may be used for isolating a pathogen and may include the steps of: contacting a library of thioaptamer beads with a labeled pathogen sample; and sorting the thioaptamer-beads that bind specifically to the labeled pathogen by flow cytometry. The library of thioaptamer beads may be a one-bead, one-aptamer library or a “library of libraries” library. The library of thioaptamer beads may be further defined as a “library of libraries” library that includes between about 100 to about 1012 beads with each bead comprising about 105 different thioaptamers. The method may also include the step of labeling the pathogen sample with a detectable marker specific to a known surface protein of the pathogen and/or washing uncomplexed sample material off the thioaptamer beads to improve the signal-to-noise ratio prior to the sorting step.

A method for identifying thioaptamers that bind to a target protein that includes the steps of: deproteinating with urea to remove bound protein from one or more thioaptamer beads that have bound specifically to the target protein and sequencing the aptamer to obtain the sequence of the aptamer on the one or more beads that bound the target protein. Yet another method of the present invention is identifying thioaptamers that bind to a target protein that includes the steps of: (a) deproteinating thioaptamers bound to a protein target with urea to remove bound protein from one or more thioaptamer beads that have bound specifically to the target protein, (b) subjecting the deproteinated beads to polymerase chain reaction (PCR) to produce a PCR product, and (c) sequencing the PCR product to obtain the sequence of the aptamer on the one or more beads that bound the target protein. The target protein may be a portion of a pathogen, e.g., a toxin, a portion of a virus, a viral surface protein, a viral envelope protein, a viral glycoprotein, a viral structural protein or portions thereof, or even an isolated protein, which may be in solution, affixed to a filter or even in a gel. Types of samples for determination may be, e.g., an environmental, an air or a biological sample. The method may also include the step of repeating steps (a)-(c). The target protein may be from a pathogen, a target cell or even a toxin or portions thereof.

Another method of the present invention may be used to determine which thioaptamers bind to a target protein that includes the steps of: (a) deproteinating thioaptamers bound to a protein target with urea to remove bound protein one or more thio-aptamer beads that have bound specifically to the target protein, (b) subjecting the deproteinated beads to PCR to produce a PCR product; (c) sequencing the PCR product to obtain the sequence of the aptamer on the one or more beads that bound the target protein; and (d) contacting a one bead-one aptamer library that includes all the aptamers the thio-aptamer beads of step (a) to identify one or more thio-aptamers that bind specifically to the target protein. Alternatively, the method for isolating a pathogen that includes the step of preparing a pathogen-specific aptamer array to screen one or more samples for the presence of a pathogen that binds specifically to the pathogen-specific aptamer array. The pathogen-specific aptamer array may include one or more isolated nucleic acid aptamers that bind specifically to a pathogen on an array, wherein a pathogen that binds to the isolated nucleic acid aptamers on the array is detected to identify the presence of the pathogen in a sample.

Yet another embodiment of the present invention is a pathogen-specific aptamer screening system that includes one or more beads that include an immobilized pathogen-specific aptamer, wherein a pathogen that binds specifically to the immobilized pathogen-specific aptamer is detected upon binding to the immobilized pathogen-specific aptamer. The pathogen may be in a sample suspected of having one or more pathogens. The one or more beads are generally contacted with a sample suspected of including one or more pathogens and the beads are sorted based on the pathogen binding to the aptamer, e.g., sorted manually, magnetically and/or by an automated bead sorting apparatus, e.g., a flow cytometer. For detection, the sample suspected of including one or more pathogens may be labeled with a detectable label, e.g., a fluorescent and/or a radiolabel. The system may include a labeled sample suspected of having one or more pathogens, wherein the pathogens are contacted with the beads; and a bead isolation system for separating the beads that that have bound a pathogen from those that have not.

In yet another embodiment, the system includes evaluating a labeled sample suspected of having one or more pathogens, wherein the pathogens are contacted with the beads; a bead isolation system for separating the beads that that have bound a pathogen from those that have not; and the step of determining the identity of the pathogen bound to a sorted bead by mass spectrometry analysis, e.g., SELDI-MS. MALDI-TOF, direct sequencing and the like. Yet another embodiment of the system includes a labeled sample suspected of including one or more pathogens, wherein the pathogens are contacted with the beads, a bead isolation system for separating the beads that that have bound a pathogen from those that have not; and identification of the pathogen bound to a sorted bead by cloning and sequencing of the aptamer. Alternatively, the system may include identification of the pathogen bound by sequencing of the pathogen.

Yet another embodiment is a system and method for concentrating a pathogen out of a sample that includes the steps of: immobilizing a pathogen-specific aptamer on a substrate and exposing a sample suspected of having the pathogen to the immobilized aptamers. The sample may be, e.g., an environmental sample and/or a biological sample, e.g., a water, a soil, a waste and an air sample or mixtures thereof. After concentration, the pathogen may be identified by, e.g., ELISA or any other detection method. The method may further include the step of determining the identity of the pathogen bound to the pathogen-specific aptamer by SELDI-mass spectrometry analysis and/or identifying the pathogen bound to the pathogen-specific aptamer by cloning and sequencing of the aptamer. Alternatively, but not mutually exclusive, the system and method may include the steps of: deproteinating the pathogen bound to the pathogen-specific aptamer and identifying the pathogen bound by, e.g., cloning and sequencing of the pathogen. Binding of the target molecule of a pathogen may be detected using, e.g., surface plasmon resonance, a capacitance coupled device or even detection of the aptamer, thioaptamer and/or the pathogen on a substrate, e.g., a semiconductor, a semiconductor array and/or a multi-well plate. Sequencing may occur before or after amplification.

Yet another method for identifying a pathogen may include the steps of: immobilizing a pathogen-specific aptamer on a substrate, exposing a sample suspected of including at least a portion of the pathogen to the immobilized aptamers and identifying the aptamer that bound the portion of the pathogen, wherein the aptamer is specific to at least a portion of the pathogen.

Another embodiment is a filter for concentrating a pathogen that includes a pathogen-specific aptamer immobilized on a substrate, wherein a sample suspected of having the pathogen is exposed to the immobilized pathogen-specific aptamers to concentrate the pathogen. Examples of substrates for use with the present invention include, e.g., a paper filter, a silicon-based material, a capacitance-coupled device and/or a three-dimensional matrix, which may also be a hydrophobic material and/or a hydrophilic material. To concentrate the pathogen, a pathogen-specific partially thio-modified aptamer is immobilized on a substrate and the sample suspected of having a pathogen is exposed to the immobilized pathogen-specific aptamers to concentrate the pathogen. The pathogen-specific aptamer may be selected from SEQ ID NOS.: 30 to 107.

Yet another embodiment of the present invention is a cancer-specific aptamer and screening system that includes one or more beads that include an immobilized cancer-specific aptamer, wherein at least a portion of a cancer cell that binds specifically to the immobilized cancer-specific aptamer is detected. The one or more beads are contacted with a sample suspected of having one or more cancer cells and the cancer-specific aptamer is isolated. For example, to prevent non-specific binding (that is binding to non-cancer cells), one or more normal cells or normal cell extracts may be bound to the aptamer library prior to binding to the cancer cells or extracts thereof. To isolate cancer cells the method disclosed herein includes contacting one or more cancer-specific thioaptamer beads with a sample suspected of having one or more labeled cancer cells followed by sorting the beads based on detection of the labeled cancer cell and/or a labeled aptamer. For sorting it may be useful to use an automated bead sorting system, e.g, a flow cytometer that may detect, e.g., a fluorescent, metallic, magnetic or even a radio label. After contacting a sample suspected of having one or more cancer cells with the beads and separation using a bead isolation system for separating the beads that have bound a portion of the cancer cells from those that have not, the present system may also include one or more of the following: a determination of the identify of the portion of the cancer cells bound to a sorted bead by mass spectrometric (SELDI) analysis, the identification of the cancer cells bound to a sorted bead by cloning and sequencing of the aptamer and/or deproteination of the cancer cells on the one or more beads followed by identification of the aptamer bound by cloning and sequencing of the aptamer.

Yet another embodiment of the present invention is a thioaptamer that is a cancer-specific thioaptamer, wherein the aptamer binds to a specific cancer cell aptamer epitope. The cancer-specific thioaptamer may further include a label, e.g., a radiolabel, a fluorescent label, a toxin, an aptazyme or aptazyme sequence, an energy-absorbing molecule or even an ionophore. The thioaptamer may be provided alone or in combination with, e.g. a potentiator such as procodazole, triprolidine, propionic acid, monensin, an anti-sense inhibitor of the RAD51 gene, bromodeoxyuridine, dipyridamole, indomethacin, a monoclonal antibody, an anti-transferrin receptor immunotoxin, metoclopramide, 7-thia-8-oxoguanosine, N-solanesyl-N,N′-bis(3,4-dimethoxybenzyl)ethylenediamine, N-[4[(4-fluorphenyl)sulfonyl]phenyl]acetamide, leucovorin, heparin, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative having potentiator activity, dimethyl sulfoxide and mixtures thereof.

For use in therapeutic uses, a formulation with a cancer-specific thioaptamer may further include a liposome, and/or a pharmaceutically acceptable salt. The formulation may be lyophilized, in injectable form and may even include one or more thio-modified nucleotides, or even partially dithio-modified nucleotides.

Another example is a pharmaceutical composition that includes a therapeutically effective amount of a thioaptamer specific for a target molecule, which may be a pathogen or pathogen-infected cell, e.g., a virally-infected cell may be targeted with an aptamer having SEQ ID NOS.: 30 to 107, or mixtures thereof. In one specific example, the aptamer is specific for a flavivirus-infected cell and may have the sequence 5′-GGC CTG TAC ACG TGT ACA CC-3′ (SEQ ID NO.: 1).

The formulations of the present invention may be used in a method of treatment with a thioaptamer that includes the step of identifying a patient in need of thioaptamer therapy and providing a therapeutically effective amount of the thioaptamer to the patient. For example, the patient may be suspected of being infected with a pathogen. If the thioaptamer is being used to treat a pathogen infected cell, the method of treating a target cell with a thioaptamer will include the step of contacting a target cell with a thioaptamer, wherein the thioaptamer binds specifically to a thioaptamer target within the target cell to modify the physiology of the target cell, e.g., to affect the physiology of the cellular defense mechanism and/or of the replication of the pathogen itself. The thioaptamer may further include an agent that leads to the death of the target cell by any number of known mechanisms, e.g., loss of cellular integrity to triggering apoptosis. The target cell may even be a cancer cell. For use in directed cellular killing, the agent may be: Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins, a radiologic agent, mixtures thereof or active subunits thereof. When used to target a cancer cell, the thioaptamer may target a prostate, breast, colon, lung, pancreatic, throat, liver, ovarian, skin or lymphoid cancer cell.

The present invention also includes kits for use of the compositions, methods and systems of the present invention. For example, one kit may be used for detecting a pathogen, the kit including a pathogen-specific aptamer and instructions for use of the pathogen-specific aptamer to detect a binding target. The binding target may be a protein, a glycoprotein, a lipid, a carbohydrate, a nucleic acid or combinations thereof from, e.g., a pathogen such as a virus, a prokaryote or a eukaryote. The binding target may be a portion of the pathogen or even be a protein produced by a host (e.g., a host cell) in response to infection by the pathogen, whether or not infectious disease occurs or is apparent. The aptamer may be a thioaptamer and may even be a concatenated aptamer that may include one or more thioaptamers. In one embodiment, the aptamer is immobilized on a substrate. The kit may further include a pathogen-specific antibody with, e.g., a detectable label. Alternatively, the kit may include a second pathogen-specific agent that includes a detectable label, in one case the detection is of the pathogen itself or of the first or second binding protein. The pathogen-specific aptamer may be selected from SEQ ID NOS.: 30 to 107.

Yet another kit may be used for concentrating a pathogen from a sample, the kit including: one or more immobilized pathogen-specific thioaptamers; and instructions for use of the one or more immobilized pathogen-specific thioaptamers. Alternatively, the kit may use an immobilized pathogen-specific aptamer on a substrate; and instructions for use of the immobilized pathogen-specific aptamer to concentrate the pathogen, wherein the pathogen-specific aptamer may bind to, e.g., a protein, a glycoprotein, a lipid, a carbohydrate, a nucleic acid or combinations thereof of the pathogen. The pathogen-specific aptamer may be selected from SEQ ID NOS.: 30 to 107. Examples of substrates for use with the kit include a paper, a hydrophobic filter, a hydrophilic filter, a semiconductor, a metal, a bead, a glass, a plastic plate, a multi-well plate, combinations thereof or other materials as will be known to the skilled artisan.

Yet another kit of the present invention may be used for identifying a target-specific aptamer and may include a thioaptamer bead library; and instructions for isolating one or more of the thioaptamer beads from the thioaptamer bead library after contacting the thioaptamer beads to an thioaptamer binding target. A kit is also disclosed for treating an infection that includes a vial that includes one or more pathogen-specific thioaptamers in a pharmaceutically acceptable form; and instructions for use of the pathogen-specific thioaptamers to treat the infection. A kit is also disclosed for treating a cancer that includes: a vial with one or more cancer cell-specific thioaptamers in a pharmaceutically acceptable form; and instructions for use of the cancer-specific thioaptamers to treat a cancer. As discussed hereinabove, the thioaptamers for use in treating an infection or even a cancer may include one or more agents that are used for direct or indirect cell killing and or potentiation. In yet further embodiments, the thioaptamers of the present invention may even be used for cosmetic therapy, wherein unwanted cells are killed or target proteins are disabled by a cell-specific thioaptamer that is conjugated to an agent that causes cell death, e.g., a toxin subunit or a small molecule, e.g., a potentiator molecule.

The present invention provides a number of advantages due to the use of modified thioaptamers and combinatorial selection methods. The present invention provides very high affinity—nM to sum-nM (≧monoclonal IgMs and >non-substituted aptamers), target-specific aptamers, demonstrating single protein target binding within cellular extracts. The modified thioaptamers have greater resistance to cellular or serum nuclease degradation than normal backbone aptamers, or proteases towards antibodies. Due to the increased nuclease resistance, the aptamers disclosed herein may be packaged to have indefinite shelf-life, ease of storage as lyophilized powders at room temperatures, unlike unmodified RNA or antibodies and are relatively inexpensive to produce. Furthermore, the methods and compositions disclosed herein allow for high reproducibility in quality control, unlike diasteromeric mixtures for non-stereospecifically produced monothiophosphate aptamers, or protein production of antibodies. Finally, the use of bead-based thioaptamer libraries or library of libraries provides large combinatorial libraries readily selected by multicolor flow cytometry at very high speeds (10⁸/hr).

The system and method may also include the step of separating the protein into fragments prior to separation by liquid chromatography followed by mass spectrometry. In an alternative method, the step of identifying the protein by mass spectrometry (MS) may be, e.g., time-of-flight (TOF) MS. In one example, prior to the step of identifying the protein, the protein may be extracted and then separated by liquid chromatography. The identification of the protein may be by surface enhanced laser desorption ionization (SELDI) or matrix assisted laser desorption ionization (MALDI) prior to MS. The thioaptamers may be attached to beads or a substrate, e.g., a semiconductor substrate. Semiconductor substrates may be used as arrays that permit detection of protein:thioaptamer binding and may further include detectors that are integral with the substrate (e.g., capacitance coupled devices) or even surface metal for surface plasmon resonance (SPR) detection. The thioaptamer library may even be a microarray on a substrate that does not include an integral detected, e.g., a glass slide on which a thioaptamer library has been disposed using, e.g., photolithography or digital optical chemistry. The location of protein binding on such a microarray may be detected using well known protein detection methods, e.g., fluorescence. The protein for use with the invention may be protein from a crude extract or even partially purified or isolated, e.g., one or more proteins isolated from a gel.

The system and method disclosed herein may further include the use of binding the thioaptamers to beads and sorting the beads to isolate and identify proteins that have specifically bound to the thioaptamers. For example, when using a thioaptamer library of beads, the beads may be sorted based on protein binding, e.g., based on fluorescence labeling of the aptamer and/or the protein using a flow-cytometer. The protein may be from a cell extract, which may even be a cell extract from a virally infected or diseased cell. Generally, the thioaptamers are attached to beads and the beads are substantially protein-free. When using a one-bead, one-thioaptamer (ODN) library or even a library of libraries the thioaptamers may be one or more beads that include an [S]—ODN and/or [S2]-ODN combinatorial libraries. The ODNs may be single or double stranded and may include thio-modifications to one or both of the strands.

In one embodiment of the present invention the thioaptamet library includes, or is designed to include, sequence motifs for high affinity with pathogen proteins. In operation, the system and method may also include the step of comparing a first and a second incubation of one or more beads to a first and a second sample, respectively, wherein differences in binding are used to detect proteins that expressed differentially, e.g., proteins from a virally-infected (or diseased) cell or even a cancer cell. In an alternative embodiment, the method may also include the steps of binding the one or more thioaptamers to one or more beads, incubating the one or more thioaptamer beads with a cell extract from a cell wherein proteins from the cell extract are labeled with a first dye; incubating the one or more thioaptamers beads with a cell extract from a diseased-cell wherein proteins from the diseased-cell extract are labeled with a second dye, incubating the one or more thioaptamers beads with a cell extract from a diseased-cell pre-treated with thioaptamers or other drugs, wherein the proteins of the diseased-cell but drug-treated, are labeled with a third dye; and performing a three-color flow cytometry that measured the relative levels of the first, second and third dyes.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows survival curves following Pichinde P18 infection in guinea pigs treated with the NF-κB aptamer, XBY-6, the scrambled control, B92, or vehicle, MT, of animals infected by injection of 1000 pfu of Pichinde P18 at day 0, treatment included intraperitoneal injections at days 0, 1 and 2;

FIG. 2 is a graph that shows survival curves of guinea pigs with thioaptamers for infection by arenavirus; and

FIG. 3 is a graph that shows survival curves following West Nile Virus infection in guinea pigs treated with the NF-κB aptamer XBY-6, the AP-1 aptamer XBY-S2, or the liposome vehicle of animals infected by injection with lethal doses of West Nile Virus.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “synthesizing” of a random combinatorial library refers to chemical methods known in the art of generating a desired sequence of nucleotides including where the desired sequence is random. Typically in the art, such sequences are produced in automated DNA synthesizers programmed to the desired sequence. Such programming can include combinations of defined sequences and random nucleotides.

“Random combinatorial oligonucleotide library” means a large number of oligonucleotides of different sequence where the insertion of a given base at given place in the sequence is random. “PCR primer nucleotide sequence” refers to a defined sequence of nucleotides forming an oligonucleotide which is used to anneal to a homologous or closely related sequence in order form the double strand required to initiate elongation using a polymerase enzyme. “Amplifying” means duplicating a sequence one or more times. Relative to a library, amplifying refers to en masse duplication of at least a majority of individual members of the library.

As used herein, “thiophosphate” or “phosphorothioate” are used interchangeably to refer analogues of DNA or RNA having sulphur in place of one or more of the non bridging oxygens bound to the phosphorus. Monothiophosphates or phosphoromonothioates [αS] have only one sulfur and are thus chiral around the phosphorus center. Dithiophosphates are substituted at both oxygens and are thus achiral. Phosphoromonothioate nucleotides are commercially available or can be synthesized by several different methods known in the art. Chemistry for synthesis of the phosphorodithioates has been developed by one of the present inventors as set forth in U.S. Pat. No. 5,218,088, issued to Gorenstein, D. G. and Farschtschi, N., issued Jun. 8, 1993, for a Process for Preparing Dithiophosphate Oligonucleotide Analogs via Nucleoside Thiophosphoramidite Intermediates, relevant portions incorporated herein by reference.

“Modified” is used herein to describe oligonucleotides or libraries in which one or more of the four constituent nucleotide bases of an oligonucleotide are analogues or esters of nucleotides normally comprising DNA or RNA backbones and wherein such modification confers increased nuclease resistance. Thiophosphate nucleotides are an example of modified nucleotides. “Phosphodiester oligonucleotide” means a chemically normal (unmodified) RNA or DNA oligonucleotide. Amplifying “enzymatically” refers to duplication of the oligonucleotide using a nucleotide polymerase enzyme such as DNA or RNA polymerase. Where amplification employs repetitive cycles of duplication such as using the “polymerase chain reaction”, the polymerase may be, e.g., a heat stable polymerase, e.g., of Thermus aquaticus or other such polymerases, whether heat stable or not.

“Contacting” in the context of target selection means incubating a oligonucleotide library with target molecules. “Target molecule” means any molecule to which specific aptamer selection is desired. “Target protein” means any peptide or protein molecule to which a specific aptamer selection is desired. “Essentially homologous” means containing at least either the identified sequence or the identified sequence with one nucleotide substitution. “Isolating” in the context of target selection means separation of oligonucleotide/target complexes, preferably DNA/protein complexes, under conditions in which weak binding oligonucleotides are eliminated.

By “split synthesis” it is meant that each unique member of the combinatorial library is attached to a separate support bead on a two (or more) column DNA synthesizer, a different thiophosphoramidite or phosphoramidite is first added onto both identical supports (at the appropriate sequence position) on each column. After the normal cycle of oxidation (or sulfurization) and blocking (which introduces the phosphate, monothiophosphate or dithiophosphate linkage at this position), the support beads are removed from the columns, mixed together and the mixture reintroduced into both columns. Synthesis may proceed with further iterations of mixing or with distinct nucleotide addition.

Aptamers may be defined as nucleic acid molecules that have been selected from random or unmodified oligonucleotides (“ODN”) libraries by their ability to bind to specific targets or “ligands.” An iterative process of in vitro selection may be used to enrich the library for species with high affinity to the target. The iterative process involves repetitive cycles of incubation of the library with a desired target, separation of free oligonucleotides from those bound to the target and amplification of the bound ODN subset using the polymerase chain reaction (“PCR”). The penultimate result is a sub-population of sequences having high affinity for the target. The sub-population may then be subcloned to sample and preserve the selected DNA sequences. These “lead compounds” are studied in further detail to elucidate the mechanism of interaction with the target.

“Detectable labels” are compounds and/or elements that can be detected due to their specific functional properties and/or chemical characteristics, the use of which allows the agent to which they are attached to be detected, and/or further quantified if desired, such as, e.g., an enzyme, an antibody, a linker, a radioisotope, an electron dense particle, a magnetic particle or a chromophore. There are many types of detectable labels, including fluorescent labels, which are easily handled, inexpensive and nontoxic.

The present inventors recognized that it is not possible to simply replace thiophosphates in a sequence that was selected for binding with a normal phosphate ester backbone oligonucleotide. Simple substitution was not practicable because the thiophosphates can significantly decrease (or increase) the specificity and/or affinity of the selected ligand for the target. It was also recognized that thiosubstitution leads to a dramatic change in the structure of the aptamer and hence alters its overall binding affinity. The sequences that were thioselected according to the present methodology, using as examples of DNA binding proteins both NF-IL6 and NF-κB, were different from those obtained by normal phosphate ester combinatorial selection.

The present invention takes advantage of the “stickiness” of thio- and dithio-phosphate ODN agents to enhance the affinity and specificity to a target molecule. In a significant improvement over existing technology, the method of selection concurrently controls and optimizes the total number of thiolated phosphates to decrease non-specific binding to non-target proteins and to enhance only the specific favorable interactions with the target. The present invention permits control over phosphates that are to be thio-substituted in a specific DNA sequence, thereby permitting the selective development of aptamers that have the combined attributes of affinity, specificity and nuclease resistance.

In one embodiment of the present invention, a method of post-selection aptamer modification is provided in which the therapeutic potential of the aptamer is improved by selective substitution of modified nucleotides into the aptamer oligonucleotide sequence. An isolated and purified target binding aptamer is identified and the nucleotide base sequence determined. Modified achiral nucleotides are substituted for one or more selected nucleotides in the sequence. In one embodiment, the substitution is obtained by chemical synthesis using dithiophosphate nucleotides. The resulting aptamers have the same nucleotide base sequence as the original aptamer but, by virtue of the inclusion of modified nucleotides into selected locations in the sequences, improved nuclease resistance and affinity is obtained.

RNA and DNA oligonucleotides (ODNs) can act as “aptamers,” (i.e., as direct in vivo inhibitors selected from combinatorial libraries) for a number of proteins, including viral proteins such as HIV RT (Burke, et al., 1996; Chen & Gold, 1994; Green, et al., 1995; Schneider, et al., 1995) and transcription factors such as human NF-κB (Bielinska, et al., 1990; Lebruska & Maher, 1999; Lin, et al., 1998; Morishita, et al., 1997; Sharma, et al., 1996). Decoy ODNs were developed to inhibit expression from CRE and AP-1 directed transcription in vivo and inhibit growth of cancer cells in vitro and in vivo (Park, et al., 1999). These studies and others (Boccaccio, et al., 1998; Cho-Chung, 1998; Eleouet, et al., 1998; Jin & Howe, 1997; Mann, 1998; Morishita, et al., 1995; Morishita, et al., 1998; Osborne, et al., 1997; Tomita, et al., 1997) have demonstrated the potential of using specific decoy and aptamer ODNs to bind to various proteins, serve as therapeutic or diagnostic reagents, and to dissect the specific role of particular transcription factors in regulating the expression of various genes. In contrast to antisense agents, duplex aptamers appear to exhibit few if any non-specific effects.

Among a large variety of modifications, S—ODN and S₂—ODN render the agents more nuclease resistant. The first antisense therapeutic drug uses a modified S—ODN (CIBA Vision, A Novartis Company). The S₂—ODNs also show significant promise, however, the effect of substitution of more nuclease-resistant thiophosphates cannot be predicted, since the sulfur substitution can lead to significantly decreased (or increased) binding to a specific protein (Milligan, J. F. and Uhlenbeck, O. C. (1989) and King et al., 2002 as well as structural perturbations (Volk, et al., 2002) and thus it is not possible to predict the effect of backbone substitution on a combinatorially selected aptamer. Hence, the present inventors recognized that selection should be carried out simultaneously for both phosphate ester backbone substitution and base sequence.

Phosphorodithioate analogs have been synthesized to produce an important class of sulfur-containing oligonucleotides, the dithiophosphate S₂—ODNs. These dithioates include an internucleotide phosphodiester group with sulfur substituted for both nonlinking phosphoryl oxygens, so they are both isosteric and isopolar with the normal phosphodiester link, and are also highly nuclease resistant. One group showed highly effective protection of the dithioate against degradation by endogenous nucleases after 58% backbone modification. Significantly, the S₂—ODNs, in contrast to the phosphoramidite-synthesized monothiophosphate (S—ODNs), are achiral about the dithiophosphate center, so problems associated with diastereomeric mixtures (Lebedev & Wickstrom, 1996) are completely avoided. The S₂—ODNs and the S—ODNs, are taken up efficiently by cells, especially if encapsulated in liposomes.

Thiophosphate aptamers are capable of specifically and non-specifically binding to proteins. Importantly, it has been observed by the present inventors that sulfurization of the phosphoryl oxygens of oligonucleotides often leads to their enhanced binding to numerous proteins (Gorenstein, 1994). The dithioate agents, for instance, appear to inhibit viral polymerases at much lower concentrations than do the monothiophosphates, which in turn are better than the normal phosphates, with K_{d}'s for single strand aptamers in the nM to sub-nM range for HIV-1 RT (Marshall & Caruthers, 1993) and NF-κB (Yang, et al., 2002, King, et al., 2002). For HIV-1 RT, dithioates bind 28-600 times more tightly than the normal aptamer oligonucleotide or the S-analogue. Sequence is also important, as demonstrated by the observation that a 14-nt dithioate based on the 3′ terminal end of human tRNA^{Lys} (CTGTTCGGGCGCCA) (SEQ ID NO.: 2) complementary to the HIV primer binding site is a more effective inhibitor (ID₅₀=4.3 nM) than simply dithioate dC₁₄ (ID₅₀=62 nM) by an order of magnitude (Marshall & Caruthers, 1993).

Oligonucleotides with high monothio- or dithiophosphate backbone substitutions appear to be “stickier” towards proteins than normal phosphate esters, an effect often attributed to “non-specific interactions.” One explanation for the higher affinity of the thiosubstituted DNAs is the poor cation coordination of the polyanionic backbone (Cho, et al., 1993, Volk, et al., 2002) sulfur, being a soft anion, does not coordinate as well to hard cations like Na⁺, unlike the hard phosphate oxyanion. The thiosubstituted phosphate esters then act as “bare” anions, and since energy is not required to strip the cations from the backbone, these agents appear to bind even more tightly to proteins.

As used herein, the terms “thio-modified aptamer” and “thioaptamer” are used interchangeably to describe oligonucleotides (ODNs) (or libraries of thioaptamers) in which one or more of the four constituent nucleotide bases of an oligonucleotide are analogues or esters of nucleotides that normally form the DNA or RNA backbones and wherein such modification confers increased nuclease resistance. For example, the modified nucleotide aptamer can include one or more phosphorothioate or phosphordithioate linkages selected from dATP(αS), dTTP(αS), dCTP(αS), dGTP(αS), dATP(αS₂), dTTP(αS₂), dCTP(αS₂) and dGTP(αS₂). In another example, no more than three adjacent phosphate sites of the modified nucleotide aptamer are replaced with phosphorothioate groups. In yet another example, at least a portion of non-adjacent dA, dC, dG, or dT phosphate sites of the modified nucleotide aptamer are replaced with phosphorothioate groups. In another example of a thioaptamer, all of the non-adjacent dA, dC, dG, or dT phosphate sites of the modified nucleotide aptamer are replaced with phosphorothioate groups; all of the non-adjacent dA, dC, dG, and dT phosphate sites of the modified nucleotide aptamer are replaced with phosphorothioate groups; or substantially all non-adjacent phosphate sites of the modified nucleotide aptamer are replaced with phosphorothioate groups. In still another embodiment of the present invention, no more than three adjacent phosphate sites of the modified nucleotide aptamer are replaced with phosphorodithioate groups. The thioaptamers may be obtained by adding bases enzymatically using a mix of four nucleotides, wherein one or more of the nucleotides is a mix of unmodified and thiophosphate-modified nucleotides, to form a partially thiophosphate-modified thioaptamer library. In another example of “thioaptamers” these are made by adding bases to an oligonucleotide wherein a portion of the phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides.

Dosage forms. A dosage unit for use of the aptamers and partially thioaptamers of the present invention, may be a single compound or mixtures thereof with other compounds, e.g., a potentiator. The compounds may be mixed together, form ionic or even covalent bonds. The aptamers and partially thioaptamers of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the aptamers and partially thioaptamers of the present invention to a patient in need of therapy that includes the aptamers and partially thioaptamers. The aptamers and partially thioaptamers may also be administered as any one of known salt forms.

Aptamers and partially thioaptamers is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the aptamers and partially thioaptamers may be formulated to provide, e.g., maximum and/or consistent dosing for the particular form for oral, rectal, topical, intravenous injection or parenteral administration. While the aptamers and partially thioaptamers may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions incorporated herein by reference.

For example, the aptamers and partially thioaptamers may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol,mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The aptamers and partially thioaptamers may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The aptamers and partially thioaptamers may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the aptamers and partially thioaptamers may be coupled one or more biodegradable polymers to achieve controlled release of the aptamers and partially thioaptamers, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the aptamers and partially thioaptamers and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions or even for delivery via a suppository. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

Intranasal and Nasal. For direct delivery to the nasal passages, sinuses, mouth, throat, esophagous, tachea, lungs and alveoli, the aptamers and partially thioaptamers may also be delivered as an intranasal form via use of a suitable intranasal vehicle. For dermal and transdermal delivery, the aptamers and partially thioaptamers may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of aptamers and partially thioaptamers may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

Effervescent tablets. To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration. Parenteral and intravenous forms may also include minerals and other materials to make them compatible with the type of injection or delivery system chosen.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

Mini-tabs. For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of pathogenic infection or even a cancer. The kit will generally include one or more containers containing a pharmaceutical composition with a therapeutically effective amount of the aptamers and/or partially thioaptamers disclosed herein. Such kits may further include one or more of various conventional pharmaceutical kit components, e.g., containers with one or more pharmaceutically acceptable diluents, as will be readily apparent to those skilled in the art. Printed instructions, either as inserts or as labels, indicating quantities of the components to be administered, guidelines for mixture and/or administration, may also be included in the kit.

The aptamers and partially thioaptamers and, optionally, one or more potentiators may be mixed with a pharmaceutically acceptable carrier. The carrier may be a solid or liquid and the type is generally chosen based on the type of administration being used. The active agent may be coadministered in the form of a tablet, capsule, liposome, as an agglomerated powder, in a liquid form or as a suppository.

Vaccines. The present invention includes vaccines for both active and passive immunization. Immunogenic compositions, suitable for use as a vaccine, include the modified thioaptamers of the present invention. The thioaptamers are prepared in a manner disclosed herein. The vaccines disclosed herein are not the antigenic material, that is, they are not intended to cause an immune response, but rather, are include either alone or in combination with an antigen to “drive” or modify an immune response by altering the activity of nuclear binding proteins, including, e.g.: NF-ATs, AP-1s, NF-IL6, NF-κB, HIV reverse transcriptase, Venezuelan Equine Encephalitis nucleocapsid (using an RNA thioaptamer), HepC IRES nucleic acid, protein(s) involved in CpG-induced “innate immunity,” and the like. As known to those in the immunological arts, the type of immunity, e.g., innate and/or adaptive, that is activated (or deactivated) is a critical step in the immune response. As such, the thioaptamers may be under some circumstances acting as an adjuvant but in others will actually be a direct participant in the immune response alone, that is, without addition of an antigen. The thioaptamers may even be used to prime the immune system prior a challenge.

In operation, the thioaptamer will generally be extensively dialyzed to remove undesired small molecular weight molecules and/or lyophilized for more ready formulation into a desired vehicle. The preparation of vaccines that include normal antigens are generally well understood in the art, as exemplified by U.S. Pat. Nos. 4,608,251; 4,601,903; 4,599,231; 4,599,230; 4,596,792; and 4.578,770, relevant portions of these incorporated herein by reference. Typically, such vaccines are prepared as injectables. Either as liquid solutions or suspensions: solid forms suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants which enhance the effectiveness of the vaccines.

In vaccine form the thioaptamer may be administered, e.g., parenterally, by injection, for example, subcutaneously, intraperitoneally, intranasally or into the lungs or even intramuscularly. Additional formulations that are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of 0.5% to 10%, or even 1-2%. Oral formulations include such normally employed excipients as, for example, pharmaceutical grades of mannitol, lactose, starch, magnesium stearate, sodium saccharine, cellulose, magnesium carbonate, mixtures thereof and the like. These compositions take the form of solutions, suspensions, tablets, pills, capsules, sustained release formulations or powders and contain 10-95% of active ingredient, preferably 25-70%.

The thioaptamers may be administered directly to the aerodigestive system (the pulmonary system and/or digestive tract) of a patient by an inhaled aerosol. Delivery of drugs or other active ingredients directly to a patient's lungs provides numerous advantages including: providing an extensive surface area for drug absorption; direct delivery of therapeutic agents to the disease site in the case of regional drug therapy; reducing the possibility of drug degradation in the patient's intestinal tract (a risk associated with oral administration); and eliminating the need for repeated subcutaneous injections. Furthermore, delivery of the thioaptamers to the pulmonary system via aerosol inhalation may be used to deliver drugs systemically, as well as for targeted local drug delivery for treatment of respiratory ailments such as pathogenic infections (viral, bacterial and fungal) or even lung cancer or asthma. Aerosol devices for use with the present invention in the clinical context include metered dose inhalers, dry powder inhalers, nebulizers and the like.

The thioaptamers may be formulated into the vaccine as neutral or salt forms. Pharmaceutically acceptable salts include those that are formed with inorganic acid, e.g., sodium, potassium, ammonium, calcium, or ferric hydroxides, and such organic bases as isopropylamine, trimethylamine, 2-ethylamino ethanol, histidine, procaine, mixtures thereof and the like. The vaccines are administered in a manner compatible with the dosage formulation, and in such amount as will be therapeutically effective. The quantity to be administered depends on the subject to be treated, including, e.g., the capacity of the individual's immune system to activate an innate immune response, synthesize antibodies or mount an effective cytotoxic T cell response, and the degree of protection desired. Precise amounts of active ingredient required to be administered depend on the judgment of the practitioner, however, suitable dosage ranges are of the order of a few to several hundred micrograms active ingredient per vaccination. Suitable regimes for initial administration and booster shots are also variable, but are typified by an initial administration followed by subsequent inoculations or other administrations. The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving an additional or complementary adjuvant effect for the thioaptamer may include, e.g., aluminum hydroxide or phosphate (alum), commonly used as 0.05 to 0.1 percent solution in phosphate buffered saline, admixture with synthetic polymers of sugars (Carbopol) used as 0.25 percent solution. When provided with a antigenic protein, the thioaptamer may be aggregated with the antigen and other components of the vaccine by heat treatment with temperatures ranging between 70° to 101° C. for 30 second to 2 minute periods. Examples of aggregation include reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six vaccinations, more usually not exceeding four vaccinations and one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescers, and the like. These techniques are well known and may be found in a wide variety of patents, such as U.S. Pat. Nos. 3,791,932; 4,174,384 and 3,949,064, as illustrative of these types of assays.

The thioaptamers may be used as part of a vaccine to regulate the development of Th1 or Th2 subsets in a subject or patient. In addition to in vivo modulation, the thioaptamers nay be used ex vivo to modify cells in vitro that are then administered to the subject. More particularly, the thioaptamers disclosed herein may be used to modulate the activity of a transcription factor (e.g., AP-1, NF-κB or NF-AT family members) that regulate innate or adaptive immune responses. In one example the thioaptamer modulates the development of Th1 or Th2 cells in the subject is modulated.

The thioaptamer vaccine may include more that one thioaptamer in order to modulate the activity of additional transcription factors that contribute to regulating the expression of Th1- or Th2-associated cytokines. In one embodiment, a stimulatory method includes a first thioaptamer that modulated the activity of an AP-1 protein and a second agent that modulates the activity of an NF-AT protein. The second agent may be a thioaptamer or even an antigen.

The thioaptamer and the methods disclosed herein may be used to manipulate Th1:Th2 ratios in a variety of clinical situations. For example, a thioaptamer may be provided that inhibits Th2 activation, which may be useful in allergic diseases, malignancies and infectious diseases. Conversely, the thioaptamer may be used to enhance Th2 activation for treatment of autoimmune diseases and/or to improve organ transplantation.

Even in specific protein-nucleic acid contacts, sulfurization of the internucleotide linkages can lead to enhanced binding (Marshall & Caruthers, 1993; Milligan & Uhlenbeck, 1989) (or to decreased affinity). The enhanced binding is very important, since most of the direct contacts between DNA-binding proteins and their binding sites are to the phosphate groups (Otwinowski, et al., 1988) (Chen, et al., 1998; Ghosh, et al., 1995; Muller, et al., 1995). The present invention takes advantage of this chemical “stickiness” to enhance the specificity and affinity of thio- and dithiophosphate agents for a protein target. It was necessary, however, to optimize the total number of thioated phosphates to decrease non-specific binding to non-target proteins and thus enhance only the specific favorable interactions with the target protein. Also, thiosubstitution can also perturb the structure of the duplex (Cho, et al., 1993) (Volk, et al, 2002) although monothiophosphates substituted in the DNA strand of DNA/RNA hybrids do not appear to have dramatically altered duplex structures (Bachelin, et al., 1998; Gonzalez, et al., 1995). The present invention uses sequence-based, structure-based and combinatorial methods to identify both sequences and thiophosphate substitution patterns to develop thioaptamers that retained the highest specificity and affinity in binding to target proteins. The use of partial thiophosphate substitution resulted in aptamer that were more stable in vivo.

In vitro combinatorial selection of thiophosphate aptamers may be used with the present invention. A recent advance in combinatorial chemistry has been the ability to construct and screen large random sequence nucleic acid libraries for affinity to proteins or other targets (Ekland, et al., 1995; Gold, et al., 1997; Tian, et al., 1995). The aptamer nucleic acid libraries are usually selected by incubating the target (protein, nucleic acid or small molecule) with the library and then separating the non-binding species from the bound. The bound fractions may then be amplified using the polymerase chain reaction (PCR) and subsequently reincubated with the target in a second round of screening. These iterations are repeated until the library is enhanced for sequences with high affinity for the target. However, agents selected from combinatorial RNA and DNA libraries have previously always had normal phosphate ester backbones, and so would generally be unsuitable as drugs or diagnostics agents that are exposed to serum or cell supernatants because of their nuclease susceptibility. The effect of substitution of nuclease-resistant thiophosphates cannot be predicted, since the sulfur substitution can lead to significantly decreased (or increased) binding to a specific protein (Milligan & Uhlenbeck, 1989).

The present invention have described the combinatorial selection of phosphorothioate oligonucleotide aptamers from random or high-sequence-diversity libraries, based on tight binding to the target (e.g. a protein or nucleic acid) of interest, relevant portions of which are incorporated herein by reference. An in vitro selection approach for RNA thioaptamers has also been described Ellington and co-workers (Jhaveri et al., 1998).

One approach used by the inventors is a hybrid monothiophosphate backbone. Competition assay for binding CK-1 42-mer aptamers were conducted. In standard competitive binding assays, ³²P-IgkB promoter element ODN duplex was incubated with recombinant p50 or p65 and competitor oligonucleotide. The reactions were then run on a nondenaturing polyacrylamide gel, and the amount of radioactivity bound to protein and shifted in the gel was quantitated by direct counting.

A combinatorial library was created by PCR, using an appropriate dNTP(αS) in the Taq polymerization step. A combinatorial thiophosphate duplex and single stranded (ss) libraries was screened successfully for binding to a number of different protein and nucleic acid targets, including NF-IL6, NF-κB, HIV reverse transcriptase, Venezuelan Equine Encephalitis nucleocapsid (using an RNA thioaptamer), HepC IRES nucleic acid, and others, including a protein involved in CpG-induced “innate immunity.” Briefly, a filter binding method was used that was modified to minimize non-specific binding of the S—ODNs to the nitrocellulose filters. A column method may also be used in which the target is covalently attached to a column support for separation as well. The duplex, ssDNA and/or ssRNA S—ODN's are eluted from the filter under high salt and protein denaturing conditions. Subsequent ethanol precipitation and for the duplex DNA S—ODNs, another Taq polymerase PCR thiophosphate amplification provided product pools for additional rounds of selection (for RNA thioaptamers RT and T7 polymerase were used). To increase the binding stringency of the remaining pool of S—ODNs in the library and select higher-affinity members, the KCl concentration was increased and the amount of protein in subsequent rounds was reduced as the iteration number increased. After cloning, the remaining members of the library were sequenced, which allowed for “thioselect”™ simultaneously for both higher affinity and more nuclease-resistant, “thioaptamer”™ agents. The thioselect method has been used to isolate a tight-binding thioaptamer for 7 of 7 targets tested.

NF-κB thioaptamers were created using thioselect for both in vitro thioselection as well as rational design of thioaptamers against NF-κB (Gorenstein et al., 1999 a, b; 2001, 2002; King et al., 2002). Sharma, et al. demonstrated previously effective aptamer inhibition of NF-κB activity. They further achieved inhibition of NF-κB in cell culture using S—ODN duplex decoys with NF-κB binding consensus-like sequence (GGGGACTTCC) (SEQ ID NO.: 3). The present inventors used the “CK-1” 42-mer duplex oligonucleotide identified by Sharma et al. (note: both the present inventors and Sharma et al.'s S—ODN duplex was chemically synthesized by sulfur oxidation with phosphoramidite chemistry and thus contains in principle 2⁸² or 10²⁴ different stereoisomers). The wild-type CK-1 duplex sequence contains 3 tandem repeats of a 14-mer NF-κB consensus-like sequence (5′-CCA GGA GAT TCC ACC CAG GAG ATT CCA CCC AGG AGA TTC CAC 3′) (SEQ ID NO.: 4).

S—ODN CK-1 monothioate aptamers were made because it was unlikely that the phosphodiester form is appropriate for therapeutics or diagnostics because of its short half-life in cells, cell extracts and serum. The phosphorothioate and dithioate internucleoside modifications are therefore needed. Using recombinant protein homodimers of p50, p65, and c-Rel, the present inventors confirmed that the CK-1 sequence could bind to and compete for binding to p65 homodimer, but not p50/p50, in standard electrophoretic mobility shift assays (EMSA)(data not shown). In contrast to the fully substituted phosphorothioate, the CK-1 aptamer inhibited p65/p65 and p50/p50 equally; confirming that S—ODNs with large numbers of phosphorothioate linkages are “sticky” and tend to bind proteins non-specifically. The present inventors also found that if the number of phosphorothioate linkages is decreased to only 2-4, specificity can be restored, but binding is not enhanced. Therefore, the original publications described only the specificity of the phosphodiester oligonucleotides and did not address the problem of altered specificity of the phosphorothioates.

Changing from purified recombinant proteins to cell culture and extracts, the situation is further complicated by the presence of the other cellular components, besides the presence of other naturally occurring NF-κB homo- and heterodimers. When the present inventors attempted to repeat the binding inhibition studies of others using cell extracts, unexpected difficulties were encountered. It was found that the diester form of the CK-1 aptamer does not compete effectively for NF-κB binding in cell extracts derived from two different cell lines: the 70Z pre-B cell line and the RAW 264.7 mouse macrophage-like line. The heterodimers in these cells either do not bind the CK-1 sequence tightly enough, or it is bound by other cellular components. Published reports describing CK-1 did not present data using cell extracts, perhaps due to similar difficulties (Sharma et al., 1996). Therefore, even sequences with good binding and specificity in the diester form, when fully thiophosphate-substituted, lose their sequence specificity. Thus, this stickiness makes the characterization of fully thioated aptamers in vitro not necessarily predictive of their activities in vivo.

TABLE 1

DNA Sequences from p50 Selection

Number
of

| | |
|---|---|
| Group 1 Sequences (n = 16) | Clones |

| | | |
|---|---|---|
| CTG TGT TCT TGT GCC GTG TCC C | 6/22 | (SEQ ID NO.:5) |
| CTG TGT TCT TGT GTC GTG TCC C | 4/22 | (SEQ ID NO.:6) |
| CTG TGT TCT TGT GTC GTG CCC C | 3/22 | (SEQ ID NO.:7) |
| CCG TGT TCT TGT GCC GTG TCC C | 2/22 | (SEQ ID NO.:8) |
| CCG TGT TCT TGT GTC GTG TCC C | 1/22 | (SEQ ID NO.:9) |

TABLE 2

DNA Sequences from p65 Selection

Number of

Clones

| | | |
|---|---|---|
| Group 1 Sequences (n = 8) | | |
| CGG GGT GTT GTC CTG TGC TCT CC | 7/16 | (SEQ ID NO.:10) |
| CGG GGT GTT CTC CTG TGC TCT CC | 1/16 | (SEQ ID NO.:11) |
| Group 2 Sequences (n = 4) | | |
| CGG GGT GGT GTG GCG AGG CGG CC | 2/16 | (SEQ ID NO.:12) |
| CGG GGT GGT GCG GCG AGG CGG CC | 1/16 | (SEQ ID NO.:13) |
| CGG GGT GTG CTG CTG CGG GCG GC | 1/16 | (SEQ ID NO.:14) |
| CGG GGT GTG CTG CTG CGG GCG GC | 1/16 | (SEQ ID NO.:15) |

Thioselection against NF-κB (p50:p50, p65:p65). As described in King et al. (2002) a unique thiophosphate duplex library was screened for binding to the p50 homodimer. Thioselection was repeated through 15 rounds to enrich for sequences that bind to p50 with high affinity. DNA sequences of multiple clones were analyzed from the initial, 2nd, 6^{th}, 10^{th} and 15^{th} round libraries. A striking convergence of the DNA sequences was observed by round 15. Of the 22 clones analyzed, 16 had a highly similar sequence (Table 1). A thioaptamer representing this sequence was generated by PCR amplification using a biotinylated reverse primer. Binding studies were conducted using a chemiluminescent EMSA, which uses a biotinylated thioaptamer. The biotinylated thioaptamer binds tightly to p50; the sequences are different from those obtained for in vitro combinatorial selection against p65 homodimers (Table 2). The chemically synthesized phosphorothioate aptamers are a diastereomeric mixture of both Rp and Sp configurations. The thioaptamers bind and compete for the same NF-κB site as the known promoter element IgkB (K_{d}=78.9±1.9 nM for a Rel A-selected thioaptamer, and 19.6±1.25 nM for a p50-selected thioaptamer). The normal phosphate ester backbone version of the Rel A selected aptamer binds Rel A with a K_{d} of 249.1±1.8 nM. The p50 dimer-selected chiral thioaptamer binds to p50 with affinities below 5 nM under conditions where no binding to p65 is observed. Similarly, the p65 dimer-selected chiral thioaptamer binds to p65 dimers with affinities below 5 nM under conditions where no binding to p50 is observed.

These EMSA binding studies demonstrated that the enhanced affinity can be attributed to the presence of sulfur. Collectively, these results further demonstrate the feasibility of the thioaptamer selection technology as a method for producing specific, high-affinity ligands to proteins. It was also demonstrated that the chemically synthesized (mixed diasteromer) thioaptamers bind tightly in cell nuclear extracts to both the p50:p65 heterodimer and p50:p50 homodimer. However, the enzymatically synthesized, chiral thioaptamer selected against the p50 homodimer only binds to p50:p50 in nuclear extracts (Fennawald et al, unpublished; King, et al., 2002; Gorenstein, patents pending, 1999 a, b, 2001). Remarkably, for the p50 homodimer the selection sequence appears to contain a pseudo-palindrome, suggesting that 2 dimers may be binding to the 22-mer sequence:

(SEQ ID NO.:16)

CTGTG PyT (CT) T G* T (G) TPy GTGTC CC

Dithiophosphate Aptamers Binding to Proteins. S₂—ODN CK-14 dithioate aptamers were also isolated. The CK-14 14-mer duplex was also synthesized with some strategically placed dithioate linkages (both of the non-bridging oxygens are replaced by sulfurs). As noted by the present inventors, strategic dithioate linkage ODNs have exhibit significant differences, as they have altered binding specificity, and lack the extreme “stickiness” of the fully thioated aptamer. With an increasing number of dithioate substitutions in the same sequence, binding by the S₂—ODN increases dramatically (data not shown). One of the tightest-binding dithioaptamer (XBY-6) contains 6 dithioate linkages on the two strands. Significantly, the XBY-6 aptamer also binds to a single NF-κB dimer in cell extracts (data not shown), while the standard phosphodiester ODN shows no NF-κB-specific binding in extracts. Thus, the present inventors succeeded in synthesizing a thioate backbone modification which for the first time increases the specific binding of the oligonucleotide to NF-κB above that to other cellular proteins (Yang, et al., 1999). In standard competitive binding assays, the ³²P-IgkB promoter element ODN was incubated with recombinant p65 and varying amounts of XBY decoy competitor. The relative binding ability of the unlabeled ODNs was determined by the concentration needed to compete effectively with the standard labeled ODN. XBY1 through 6 correspond to CK-14 aptamers with with 1 though 6 dithiophosphate substitutions, respectively (Yang, et al., 1999).

ODN aptamer was incubated with 70Z/3 cell nuclear extract in the presence or absence of anti-p50 antibody. Protein-bound ODN duplex was separated on a standard gel. XBY-6 shifts one complex in nuclear extracts from a 70Z/3 pre-B cell line. By using specific antibodies to supershift the complex, p50 was identified as one component of the complex, which may be a complex that include a p50 or p105 dimer, or a p50 (or p105)-containing heterodimer. Since XBY-6 binds more tightly to p50/p50 than p65/p65, the shifted band is likely to represent the p50 homodimer. The band did not co-migrate with either the p50/p50 or p50/p65 bands, but the change in the altered chemical structure changes the mobility of the ODN. Only one major band is seen, however, even though the lysate contains at least two major distinguishable NF-κB complexes (p50 homodimers and p50/p65 heterodimers).

These results demonstrate the use of aptamers having altered binding specificity and affinity by substituting only a limited number of internucleoside linkages, that is, a portion of the internucleoside linkages. The partially-modified aptamer was used to distinguish among various NF-κB dimers within the cell. The IgκB standard ODN does not show such specificity. Therefore, this modified thioaptamer may be used to bind to a single NF-κB dimer within cell supernatants and even inactivate target dimers within whole cells and animals. It was also found that when guinea pigs were injected with LPS to induce inflammatory response and XBY-6, an increase in the levels of TNF-α was observed above that when the animals were injected with LPS alone. In animal macrophage extract studies, it was found that XBY-6 eliminated a single p50 (or p105) dimer band on EMSAs. Since the p50 homodimer appears to be a transcriptional inhibitor of the immune response, these data demonstrate the ability to target a single protein within live animals, and the feasibility of altering the binding specificity by substituting only a limited number of internucleoside linkages (Gorenstein, et al. patents pending, 1999 a, b; 2001, 2002). Using the modified thioaptamer a 1:1 binding stoichiometry of p65 to the 22 mer binding site known as IgκB with a K_{d} near 4 nM. For one dithiophosphate aptamer, XBY-6, a binding affinity to p65 homodimer of 1.4 nM vs. sub-nM to p50 was demonstrated.

Various thioaptamers have been made and isolated using the present invention that can distinguish among various NF-κB dimers within the cell. One of these decoys was able to bind to a single NF-κB dimer in cell extracts or within a cell in either cell culture or animal studies. These results point to the importance of using modified thiophosphate combinatorial selection methods to identify minimally substituted thioated oligonucleotides with high affinity, high binding specificity and increased nuclease resistance in vitro and in vivo.

Phosphorodithioate and phosphorothioate aptamers via split synthesis combinatorial selection. The identification of specific S—ODN and S₂—ODN thioaptamers that bind proteins based upon in vitro combinatorial selection methods is limited to substrates only accepted by polymerases required for reamplification of selected libraries by the polymerase chain reaction (PCR). Another disadvantage of using the polymerization of substituted nucleoside 5′-triphosphates into ODN aptamers are the restrictions on the choice of P-chirality by the enzymatic stereospecificity. For example, it is known that [Sₚ]-diastereoisomers of dNTP(αS) in Taq-catalyzed polymerization solely yield [Rₚ]-phosphorothioate stereoisomers (Eckstein, 1985). Therefore, using current methods it is not possible to select [Sₚ]-phosphorothioate stereoisomers along with achiral S₂—ODN analogous since both [Rₚ]-diastereoisomers of dNTP(αS) and nucleoside dNTP(αS₂) are not substrates of polymerases. Additionally, these in vitro combinatorial selection methods require many iterative cycles of selection and reamplification of the bound remaining members of the library by the PCR, which are quite time consuming, although automation of this in vitro selection is possible.

What is needed are methods that permit the isolation of, e.g., individual aptamer:protein complexes without the need for repeated iterative cycles of selection and reamplification of likely binding targets. Also needed are systems that permit the creation, isolation, sequencing and characterization of making [Sₚ]-phosphorothioate stereoisomers along with achiral S₂—ODN analogs. To overcome these limitations of the in vitro combinatorial selection methods, the present inventors developed a one-bead, one-compound library made by using a split synthesis method to create an alternative to in vitro combinatorial selection methods. One-bead library systems have been used for organic molecules (Felder, (1999)), peptides (Lam, et al., 1991, 1995; Lam, 1995), and oligosaccharide libraries (Zhu and Boom, 1998; Liang, et al., 1996; Hilaire and Meldal, 2000). A one-bead one-oligonucleotide (one-ODN) (e.g., O—ODN, S—ODN, S₂—ODN, both DNA or RNA) may be used in conjunction with combinatorial library selection methodology used to identifying a specific oligonucleotide aptamer that binds to specific proteins or other molecules (Yang, et al., 2002; Gorenstein, et al., U.S. patent applied).

Furthermore, the method may use S₂—ODN reagents with sulfurs replacing both of the non-bridging phosphate oxygens that are isosteric and isopolar with the normal phosphorodiester and are particularly advantageous for binding and screening. Importantly, S₂—ODNs are achiral about the dithiophosphate center, which eliminated problems associated with diastereomeric mixtures generally obtained for the chemically synthesized S—ODN. The split synthesis approach disclosed herein has been used for the construction of O—ODN, S—ODN, S₂—ODN and RNA bead-based aptamer and thioaptamer libraries (Gorenstein, et al., US patents pending, 1999 a, b, 2001, 2002; awarded, 2002; Yang, et al., 2002). In this procedure each unique member of the combinatorial library is attached to a separate support bead. Targets that bind tightly to only a few of the 10⁴-10⁸ different support beads can be selected by binding the target protein to the beads and then identifying which beads have bound target by immunostaining techniques or direct staining of the target or SELDI MS (see below). The present methodology permits rapid screening and identification of modified thioaptamers that bind to proteins such as NF-κB using a novel PCR-based identification tag of the selected bead.

To introduce many copies of a single, chemically pure S—ODN thioaptamer onto each bead, a “mix and separate” split synthesis method was used. A two-column DNA synthesizer was used simultaneously for construction of the library. The normal phosphate backbone linkages were carried out using standard phosphoramidite monomers via oxidation in column 1, while the phosphorothioate linkages were carried out using standard phosphoramidite monomers via sulfurization in column 2. Dithioate are introduced by using thiophosphoramites with sulfur oxidation. Two sequences of the same length are programmed for each column and are designed such that the bases are different at every equal position not only for diversifying base compositions but also for coding a phosphate, phosphoromonothioate/dithioate.

For example, on an Expedite 8909 DNA synthesizer with dual columns, onto column 1 a phosphoramidite (for example: C) is coupled to the bead and after completion of oxidation, the resulting product is nucleotide (C) with a phosphotriester linkage. On column 2 a nucleoside phosphorothioate is introduced with a different base (T for example). The two columns are mixed and resplit and in the second cycle, additional phosphoramidites or phosphorothioamidites are introduced, followed by oxidation and sulfurization reactions individually in column 1 and column 2. After additional coupling steps and after split/pool synthesis is carried out, the end products comprise a combinatorial library of thioaptamers with varying monothioate, dithioate or normal phosphate ester linkages at varying positions along the ODN strand. On completion of the automated synthesis, the column is removed from the synthesizer and dried with argon. The bead bound fully protected ODNs are treated with 1 mL of concentrated ammonia for 1 h at room temperature, incubated in a 55° C. oven for 15-16 h, removed from the oven and cooled to room temperature. Importantly, after deprotection, with this coupling scheme with a non-cleavable hexaethyleneglycol linkers. Linker attaching the first phosphoramidite (15 or 70 μm beads provided by ChemGenes), the thioaptamers are still covalently attached to the beads after complete deprotection. Thus, each bead contains a single sequence with a specified backbone modification that is identified by the base.

For example, this scheme was used to synthesize libraries of 4096 (2¹²) different thioaptamers attached to beads, each bead containing a unique thioaptamer. This library consisted of a 22-nucleotide “random” sequence (12 split/pool steps) flanked by 15 nucleotide defined primer regions at the 5′ and 3′ ends (Yang et al., 2002). A phosphorothioate linkage was introduced on every other base in column 2, following the “split and pool” approach. The single-stranded 52-mer S—ODN random library was converted to double-stranded DNA by Klenow DNA polymerase 1 (Promega) reaction in the presence of DNA polymerase buffer, dNTP mix and downstream primer. Therefore, the one strand of the duplex potentially contained S—ODN modifications and the other complementary strand were composed of ODN. A duplex DNA library in which both strands contain S—ODN modifications could also be generated using a Klenow reaction with no more than three dNTP (α)S.

The dsDNA thioaptamer library beads were screened for the ability to bind the NF-κB p50/p50 dimer labeled with the Alexa Fluor 488 dye (Molecular Probes). After initial binding of protein, the beads were thoroughly washed with PBS with 0.1% Tween 20 to minimize nonspecific binding. Typically, a few positive beads were intensely stained when viewed by fluorescence, while the majority of the beads remained unstained as (data not shown). With the aid of a micropipette coupled to a micromanipulator, the intensely stained beads were retrieved. Only highly positive beads from several thousand were found using this method. As described below, multicolor flow cytometry and cell/bead sorting was used to automate the selection process to select the tightest binding thioaptamer-protein complexes.

Sequencing may also be obtained directly from the bead. Each individually selected bead was washed thoroughly with 8 M urea (pH 7.2) to remove the protein and was directly used for the “one-bead one-PCR” amplification using the 5′ and 3′ end primers. The PCR product was cloned using the TA Cloning procedure (Invitrogen) and sequenced on an ABI Prism 310 Genetic Analyzer (Applied Biosystems). The four thioaptamers listed in Table 3 were obtained from the library. For verification of these results, the S—ODN, 5′-CtGTGAGtCGACTgAtGaCGGt-3′ (SEQ ID NO.: 17) (small letters represent location of 3′-monothiophosphates), was independently synthesized on the non-cleavable linker bead support, hybridized with its complementary ODN and then mixed again with the p50/p50 protein labeled with the Alexa Fluor 488 dye. The fluorescence intensity of all of the beads viewed under the fluorescence microscope was qualitatively similar to the intensity of the selected bead containing this sequence within the combinatorial library. These results demonstrate that the primer regions do not contribute to the binding of p50/50. Furthermore, it was found that not only normal monothio-ODN on the beads but also dithio-modified bead-bound sequences could be sequenced directly from the dithiophosphate combinatorial library. Thus, the split synthesis has been used to create a “one-bead-one sequence” ODN and that PCR can be used to identify an S—ODN bound to a bead (Yang et al., 2002; Gorenstein et al, US & Foreign Patents pending, 1999 a, b, 2001, 2002).

Bead-based thioaptamer library screen. Aliquots of S—ODN beads bound to NF-κB p50/p50 homodimer protein labeled with the Alexa Fluor 488 dye viewed under light microscopy. The same beads viewed under fluorescence microscopy, in which a positive green bead stained with Alexa Fluor 488 dye were easily identified in a background of many hundreds of nonreactive beads. Single positive bead can easily be retrieved with a handheld micropipette under fluorescence microscopy.

Although the beads were screened against a target protein labeled with a fluorescent dye, the beads have also been screened directly against cell extracts as well. The binding of the NF-κB to a specific sequence can be detected using a primary anti-NF-κB antibody such as anti-P50 (Rabbit IgG antibody, Santa Cruz Biotechnology, Inc.) followed by a secondary antibody conjugated with Alexa Fluor 488 (goat anti-rabbit IgG from Molecular Probes). Beads that included the XBY-6 oligonucleotide were screened against WI-38 VA13, an SV40 virus-transformed human fibroblastic cell line extract by similar fluorescent microscopy.

Other bead-based thioaptamer libraries. Combinatorial thioaptamer bead libraries of over 10⁶ different sequences have also been readily prepared. The present inventors have synthesized successfully a monothio RNA library (2¹⁵=32768). Thus, standard phosphoramidite (DNA and RNA) chemistry was used for the thioaptamer RNA library. A 0.5 M 1H-tetrazole in acetonitrile was used as DNA activator. A 0.5 M solution of DCI (dicyanoimidazole) in acetonitrile was used as RNA activator. The libraries were prepared on a 1 μmole scale of polystyrene beads (66-70 μm). The downstream and upstream primers, 5′-d(GGATCCGGTGGTCTG)-3′ (SEQ ID NO.: 18) and 5′-d(CCTACTCGCGAATTC)-3′ (SEQ ID NO.: 19) were synthesized in parallel on a two-column DNA synthesizer (Expedite 8909, Applied Biosystems). Following the 5′-primer, the sequences programmed on the synthesizer for the combinatorial mono RNA library were 5′-r(GA*UC*CU*GA*AA*CU*GU*UU*UA*AG*GU*UG*GC*CG*AU*C)-3′ (SEQ ID NO.: 20) on column 1 and 5′-r(cU*aG*gA*cU*uG*gC*aC*aA*cC*gU*cA*cA*cU*gC*uA*u)-3′ (SEQ ID NO.: 21) on column 2. The 3′-primer sequence completed the 61-mer programmed on the synthesizer. A “split and pool” occurred at each position indicated by an asterisk in order to synthesize the combinatorial region for the monothio RNA. The lower case letter indicates a 3′-thioate linkage, the upper case letter indicates a 3′-phosphate linkage. The coupling yield was typically upwards of 98.5% as determined by the dimethoxytrityl cation assay (DNA couplings are typically >99%/nt). Sulfurization chemistry utilized the Beaucage reagent. The fully protected monothio RNA combinatorial library with the non-cleavable linker beads were treated with 4 ml of a mixture of 3:1 (v/v) (28%) NH₃:EtOH at 39° C. for 21 hrs. The beads were centrifuged, the supernatant was removed and the solid support was washed with double-distilled water. After lyophilization the solid support was treated with 2 ml of triethylamine trihydrofluoride (TEA-3HF) for 20 hrs at room temperature. Again, the beads were centrifuged, the supernatant was removed and the solid support was washed with double-distilled water. RT PCR and TA cloning confirmed the successful synthesis of the ssRNA thioaptamer library.

TABLE 3

Sequences of thioaptamers selected from split
synthesis (small letters indicate
thiophosphate 3′ to base).

| | |
|---|---|
| 5′-tGTGcAGGGACTgAtGaCGGt-3′ | (SEQ ID NO.:22) |
| 5′-CtGTGCatCGAaGTTtGCAtTt-3′ | (SEQ ID NO.:17) |
| 5′-AtGcAcAtCtCaGgAtGaCGGt-3′ | (SEQ ID NO.:23) |
| 5′-AGTTGcAGGtCaGgACCCAtTt-3′ | (SEQ ID NO.:24) |

Flow cytometry sorting of thioaptamer bead-based library. The present inventors have also demonstrated the successful application of high throughput/multi-color flow cytometry and bead sorting to screen aptamer bead libraries for those beads which bind to, e.g., a target protein (Gorenstein, et al., patent pending, 2002). Modifications were made to a custom-built flow cytometer to make it more amenable to bead identification and isolation. For example, bead fluorescence and forward scatter were the two parameters chosen for real-time characterization of each aptamer bead passing the first sort point of the custom-built flow cytometer/sorter. Other scanning and sorting parameters may be used to select, isolate, view, designate, characeterize, etc. the beads through a flow cytometer.

In operation, “positive” beads (contain thioaptamer-bound target protein, the target protein was fluorescent-labelled with Alexa 488 dye) were easily sorted from negative beads. Flow cytometry may be used to replace, e.g., visual fluorescence microscope identification of beads containing bound target protein and the need to isolate the individual “positive” beads with the micromanipulator described previously. The flow-sorted “positive” beads can then be subjected to, e.g., one-bead PCR to identify the thioaptamer that binds the target protein.

TABLE 4
Population Statistics for bead sorting,
WinList analyses (all data were color-compensated)

| Sample | Total | Region | % Gate |
|---|---|---|---|
| CONTROL.FCS | | | |
| R1: Autofluorescent Beads | 10000 | 9530 | 95.3 |
| R2: p50 Alexa 488 Positive Beads | 10000 | 35 | 0.35 |
| R3: p65 PE Positive Beads | 20000 | 3488 | 17.44 |
| R1: Autofl. Beads & Carrier Beads | 1000000 | 963321 | 96.33 |
| R2: p50 Alexa 488 Positive Beads | 1000000 | 354 | 0.04 |
| R3: p65 PE Positive Beads | 1000000 | 935 | 0.09 |

Fluorescence sorting was also used to demonstrate the use of the one-bead, one-ODN:protein system using dual color sorting. The IgκB dsDNA consensus sequences were immobilized onto 15-20 polystyrene microspheres. The DNA bound beads were then incubated with purified p50 and p65 proteins, respectively. DNA transcription factor complexes were detected with primary antibodies specific for the p50 and p65 proteins followed by an additional incubation with Alexa 488-conjugated secondary antibody for p50 and PE-conjugated secondary antibody for p65. The beads were viewed by fluorescent microscopy and then analyzed on the MCU's HiReCS system. A Control Fluorescent Cell Sort (CONTROL.FCS) shows the autofluorescent microspheres in the negative control sample where the beads were unbound. The majority of the beads in the “debris” population were the 0.8 micron carrier beads that were used to bring up the volume of the samples since the beads were at a very low dilution.

Innate Immunity Toll-Like Receptor Signaling. In another embodiment of this invention, the present inventors developed thioaptamers that enhance the innate immune response by targeting the Toll-like receptor (TLR) family in mammals, which is a family of transmembrane proteins characterized by multiple copies of leucine rich repeats in the extracellular domain and IL-1 receptor motif in the cytoplasmic domain (Akira, et al., 2001; Medzhitov, 2001). The TRL family is a phylogenetically conserved mediator of innate immunity that is essential for microbial recognition. Ten human homologs of TLRs (TLR1-10) have been described. By using a BLAST search, Hemmi et al., 2000, have identified and subsequently isolated a cDNA coding for TLR9. Gene knockout experiments suggest that TRL9 acts as a receptor for unmethylated CpG dinucleotides in the bacterial DNA. Human and mouse TLR9 share an overall amino-acid identity of 75.5%. TLR9 is highly expressed in spleen (Krieg, 2002).

The immunostimulatory properties of bacterial DNA appears to be related to short six base sequences called CpG motifs that have the general structure of two 5′ purines, an unmethylated CpG motif, and two 3′ pyrimidines (Krieg, 2002). Though such sequences rarely appear in mammalian DNA due to CpG suppression and methylation of cytosine nucleotides, they are relatively abundant in bacterial DNA, occurring at the expected frequency (1 in 16) and in unmethylated form. Indeed, studies have found ODNs containing these sequence motifs to be strongly immunostimulatory, resulting in the activation of B cells, NK cells, and antigen-presenting cells, and in the induction of a variety of cytokines including interleukin-12 (IL-12), IL-6, and tumor necrosis factor-α. CpG ODNs have also been found to be effective as adjuvants in inducing antigen-specific T-helper-1-like responses, and have been the focus of much interest for their inclusion in anti-tumor vaccinations and use in other therapeutic applications (Klinman, et al., 1999; Krieg, et al, 1999). Adjuvants enhance nonspecifically the immune response to an antigen. For example, pathogenic Arenaviruses appear to block or modify immunoregulatory cell signaling pathways (Peters & Zaki, 2002, Solomon and Vaughn, 2002; Fennewald, et al., 2002). Using the present invention it was possible to disrupt Arenavirus and Flavivirus cell signals that contribute to immune evasion and pathogenesis. Using thioaptamers it was demonstrated that the thio-modified aptamers of the present invention could be used to counteract viral induced cellular perturbations and protect the infected host.

Viral Strategies to manage the host. During the co-evolution of viruses and their hosts, viruses have developed ingenious strategies to counteract the host defenses that normally control viral replication and spread. Similarly, viral strategies modify the cellular environment to promote viral macromolecular synthesis and viral replication. This highly ordered interation often has the unfortunate consequence of inducing disease in the host. Viruses have evolved mechanisms to interfere with major histocompatibility complex antigen presentation, block apoptosis; disrupt complement cascades and modulate multiple cytokine networks (Lalani & McFadden, 1999; Ploegh, 1998). Viruses have targeted cell-signaling pathways involved in cytokine and chemokine signaling, the regulation of apoptosis, and the cell cycle. Studies have revealed a number of instances of direct viral intervention in the receptor and receptor proximal signaling, as well as direct interaction with signaling kinase cascades and transcription factors (McFadden, et al., 1998; Ploegh, 1998; Hiscott, 2001; Hiscott, et al., 2001). Most examples have come from large DNA viruses with sufficient coding capacity to encode viral homologs of cellular proteins. These viruses use molecular mimicry to exploit the cellular environment to promote viral replication and antagonize the immune response to sustain their survival in an immunocompetent host (Cameron, et al., 1999; Willer, et al., 1999; Hiscott, et al., 2001). Influencing key transcription factors that regulate pro or anti-inflammatory cytokines is an efficient means by which viruses could cripple multiple immune responses (Powell, et al., 1996; Tait, et al., 2000). The strategies employed by the smaller, less genetically complex viruses are equally elegant, and often even more of an enigma.

Pichinde infection of guinea pigs is particularly suited to studies on the immunomodulation by virus infection. There are two virus variants with minimal genomic differences but profoundly different effects on the animal. Infection by the P2 variant of virus results in mild illness from which the animal recovers. Infection by the P18 variant results in death. These two virus variants were used to distinguish an effective immune response against the P2 virus, from an ineffective response against the P18 virus.

Using the aptamers of the present invention, the differential effect of virus infection was identified as including a profound effect on the transcription factors NF-κB and RBP-Jκ. Data generated by the present inventors (Fennewald, et al., 2002) showed differential alterations in the transcription factors NF-κB and RBP-Jκ in P2 and P18 virus-infected guinea pig peritoneal macrophages. The P2 variant shows less NF-κB present and a higher mobility RBP-Jκ complex. This observation was used in an animal model of arenavirus disease in which two virus variants differentially affect target cell signaling pathways. NF-κB and AP-1(CREB) family members are key regulators of the immune response and transcription factors involved interferon response to virus infection all are differentially induced in pathogenic Pichinde infections. Using the aptamers of the present invention infected hosts virulence was reduced by modulating virus induced alterations in cellular signal transduction.

Many of the signaling pathways and transcription factors activated during immune system activation lead to the synthesis of the inflammatory cytokines. Certain pathways require the expression of various cytokines. The effect of the virus variants (and polyl/C) on the induction of cytokines was determined. Infection with P2 and P18 also alter the expression of this and other inflammatory cytokines. In particular, P2 and P18 induced equally cytokines such as IL-6; which are moderately different in their induction of TNF-α and substantially different in IL-12 induction (data not shown). Thus, differences in signaling and inflammatory responses are associated with immune activation by P2 virus and poor activation by the P18 virus. For example, IL-12 is especially important in directing the anti-viral immune response to the effective Th1 cytotoxic T cell response (Seow, 1998). In addition to supporting the association with the immune response, this data can be used to direct the transcription factors to target. For example, IL-6 induction is similar for both virus variants.

The thioaptamer will generally bind specifically to a protein, e.g., a transcription factor and may also include one or more of the aptamers of SEQ ID NOS.: 2, 3, 4, 5, 6, 7, 8 and 9, e.g.:

| | | |
|---|---|---|
| XBY-6: | 5′-CCAGGAGAT_{S2}T_{S2}CCAC-3′ | SEQ ID NO.:25 |
| | 3′-GG_{S2}TCC_{S2}TC_{S2}TAAGG_{S2}TG-5′ | |
| XBY-S2: | 5′-CCAGT_{S2}GACT_{S2}CAGT_{S2}G-3′ | SEQ ID NO.:26 |
| | 3′-GG_{S2}TCAC_{S2}TGAG_{S2}TCAC-5′ | |
| XBY-S1: | 5′-T_{S2}T_{S2}GCGCGCAACAT_{S2}G-3′ | SEQ ID NO.:27 |
| | 3′-AACGCGCG_{S2}T_{S2}TG_{S2}TAC-5′ | |
| XBY-C2: | 5′-CCAGTGACTCAGTG-3′ | SEQ ID NO.:28 |
| | 3′-GGTCACTGAGTCAC-5′ | |
| XBY-C1: | 5′-TTGCGCGCAACATG-3′ | SEQ ID NO.:29 |
| | 3′-AACGCGCGTTGTAC-5′ | |
| | 5′-tGTGcAGGGACTgAtGaCGGt-3′, | SEQ ID NO.:21 |
| | 5′-CtGTGCatCGAaGTTtGCAtTt-3′, | SEQ ID NO.:17 |
| | 5′-AtGcAcAtCtCaGgAtGaCGGt-3′, | SEQ ID NO.:22 |
| | 5′-AGTTGcAGGtCaGgACCCAtTt- 3′, | SEQ ID NO.:23 |

wherein the lowercase letters represent the thiophosphate 3′ to the base. In one examples, the method of treatment may be directed to a neuropathologic viral infection and include the steps of identifying a patient suspected of being infected with a neuropathologic virus; and providing the patient with a therapeutic amount of a partially thioaptamer specific for transcription factor involved in immune cell activation. A thioaptamer for use in the method of treatment may be XBY-S2.

To target transcription factors key in regulating TNFα and IL12 and other key mediators of the immune response two thioaptamers were produced, XBY-6 targeting NF-κB p50 homodimers and XBY-S2 targeting AP-1, both with six dithio residues. XBY-S2 was demonstrated to bind specifically to AP-1 proteins in pre-B cell nuclear extracts (70Z/3) and to human recombinant c-jun protein dimers (AP-1). Supershift analyses indicate that XBY-S2 binds to several members of the AP-1 protein family including JunD, CREB and possibly ATF2, and c-Jun. The XBY-6 thioaptamer binds specifically to the NF-κB p50 (or p105) homodimer. Macrophage cultures were treated with XBY-S2 and XBY-6 and nuclear extracts were produced to assay the effects of these thioaptamers on the DNA binding activities of the transcription factors to which they are targeted. Next, macrophage cultures were treated with liposomes, and liposome containing the indicated thioaptamers overnight and nuclear extracts produced and assayed using the indicated oligonucleotides. The XBY-S2 thioaptamer efficiently eliminated transcription factor binding to the AP-1 oligonucleotide. In contrast, treatment with XBY-6 resulted in an increase in the NF-κB DNA binding activity.

In order to determine the consequence of the elimination of AP-1 DNA binding activity by XBY-S2, stimulated macrophage cultures were incubated with the thioaptamer with Polyl/C and measured the elaboration of TNFα and IL-6 into culture media. The expression of both TNFα and IL-6 are increased in response to polyl/C (data not shown). Pretreatment of cultures with XBY-S2 thioaptamer increases the amount of both cytokines produced in response to poly I/C. These results indicate that elimination of AP-1 from cells by the XBY-S2 decoy thioaptamer increases the production of cytokines.

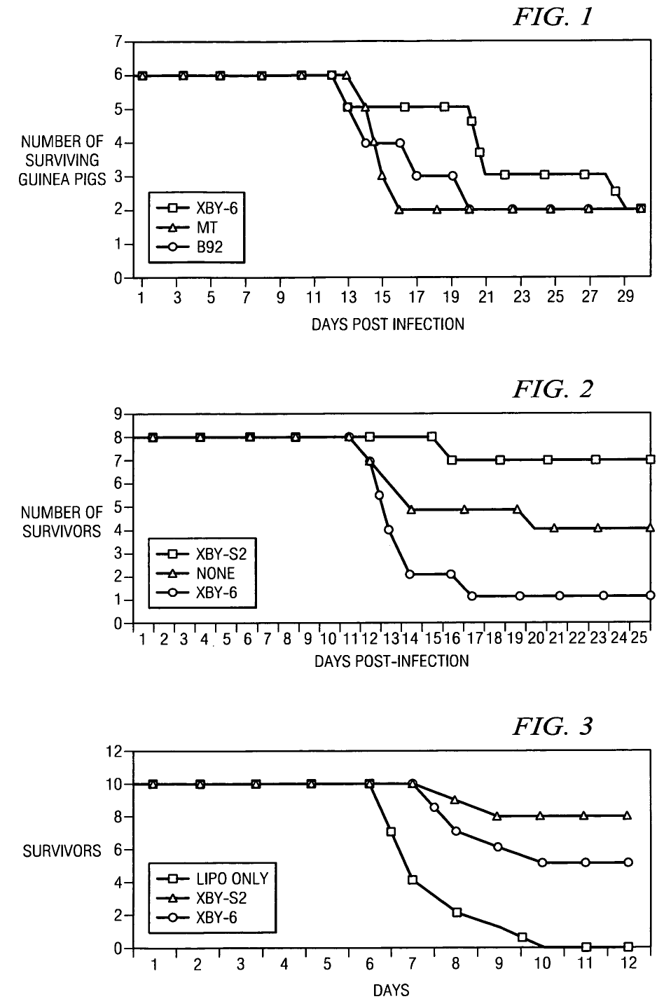

It has been suggested that arenaviral and West Nile pathogenesis is the result of viral perturbation of the immune response resulting in the inappropriate expression of cytokines. Therefore, modulation of cell signaling by appropriate thioaptamers could reverse the inappropriate gene expression and help to alleviate the symptoms and perhaps prevent host death. Guinea pigs were treated with the XBY-6 thioaptamer targeting NF-κB p50 homodimers at days 0, 1, and 2 day relative to time of infection with a lethal dose of Pichinde virus. FIG. 1 is a graph that demonstrates that the thioaptamer XBY-S2, created, isolated and characterized according to the teachings of the present invention prolongs the survival of Arenavirus infected animals. A thioaptamer of the same base content but scrambled in sequence and containing CpG islands did not prolong survival (B92; FIG. 1). Using the XBY-S2 thioaptamer, 50-80% protection of mice from a lethal West Nile virus infection was demonstrated (Tables 5 and 6) as well as prolongation of Pichinde virus survival similar to XBY-6 (data not shown).

TABLE 5
Female 3-4 week-old NIH Swiss mice were given aptamers at
one day before and 90 minutes before administration of
10 LD₅₀ WN virus strain USA99b by intraperitoneal injection.

| Group | # surviving [%] | AST (days ± SD) |
|---|---|---|
| PBS only | 0/5 [0] | 7.2 ± 0.4 |
| Liposomes only | 0/5 [0] | 8.0 ± 0.7 |
| XBY-S2 | 4/5 [80] | 9 |
| XBY-6 | 4/5 [80] | 11 |

Based on the preliminary results obtained with XBY-6 thioaptamer and Pichinde virus, it was determined if XBY-6 or XBY-S2 would have any antiviral activity against flaviviruses. West Nile virus was selected as a model system due to its high virulence in the mouse model. Mice were challenged with a low dose of virus (i.e., 30 pfu≈10 LD₅₀). The thioaptamers (10 μg) were delivered IP in Tfx50 liposomes and administered in two doses (one day before and 90 minutes before virus challenge). Control mice given PBS or liposomes succumbed to WN virus infection, while 80% of thioaptamer XBY-S2 treated animals survived challenge and remained healthy (Table 5). It was noted that both thioaptamers had antiviral activity. These results suggested that while the mechanism of protection may involve binding of XBY-6 to NF-κB or XBY-S2 to AP-1.

In previous studies with West Nile virus the present inventors had observed hat animals had a brief viremia that peaked on day 3 post infection prior to viral brain invasion. As such, three animals from each test group were sacrificed on days 3 and 6 post infection to determine viremias and virus infectivity levels in the brain. Accordingly, the protocol from the first study was repeated with increased group sizes of 16 mice (of which 6 would be sampled) and increasing the virus challenge to 100 LD₅₀ virus. As shown in Table 6, the initial results were reproducible. Both control groups (PBS and liposomes) succumbed to challenge with WN virus while the thioaptamer-treated mice survived and remained healthy. The proportion of mice treated with XBY-S2 thioaptamer who survived challenge was the same in both studies (80%) while XBY-6 treatment protected 50% of mice in the second study as compared to 80% of mice in the first study. These differences were not statistically significant given the small sample sizes.

To obtain fundamental information on the mechanism of protection, viremias and brain infectivity titers were measured in three mice sampled from each group on days 3 and 6 post infection (Table 7). As expected, viremias and brain infectivity titers in the control (PBS and liposome) groups detected on day 3 prior to invasion of the brain and virus detectable in the brains on day 6 post infection. The thioaptamer treated mice had reduced or undetectable viremias on day 3 post infection and no detectable virus infectivity in brains on day 6 post infection. These data indicate that the thioaptamer causes a reduction in the extraneuronal replication of the virus (as seen in the reduced viremias) and that there is insufficient virus to invade the central nervous system and cause encephalitic disease. The difference between virulent neuroinvasive strains of WN virus and poorly neuroinvasive attenuated WN strains may be explained by these results. Two mechanisms seem possible, although the invention is in no way limited by hypothesis: 1) first, the thioapiamer induces an immune response against WN virus; or 2) the thioaptamer blocks the WN virus replication. The thioaptamer may be inducing localized interferon (or other mediators of the innate immune response) that inhibits replication of the virus since the thioaptamer includes double-stranded DNA while double-stranded RNA is known to be an efficient inducer of interferon.

TABLE 6
Study 2: Female 3-4 week-old NIH Swiss mice were given
aptamers at one day before and 90 minutes before administration
of 100 LD₅₀ WN virus strain USA99b by the ip route.

| Group | # surviving [%] | AST (days ± SD) |
|---|---|---|
| PBS only | 0/10 [0] | 8.3 ± 0.8 |
| Liposomes only | 0/10 [0] | 7.7 ± 1.1 |
| XBY-S2 | 8/10 [80] | 8.5 ± 0.7 |
| XBY-6 | 5/10 [80] | 8.0 ± 0.7 |

To investigate the activity of the modified thioaptamers and the antiviral mechanism of action of the thioaptamers, the susceptibility of thioaptamer-protected mice virus to challenge was tested. Thioaptamer-treated mice from the second study who survived WN virus infection were challenged at 21 days post-infection with 100LD₅₀ of WN virus. All mice, including mock-infected controls from study 2 succumbed to virus challenge. This result indicates that there was insufficient virus replication in thioaptamer-treated mice to induce an adaptive immune response. This would suggest that the mechanism of action of the thioaptamer is either innate immunity or direct antiviral activity of the thioaptamer.

TABLE 7
Viremia and brain infectivity titers for Study 2

| | Day 3 | | Day 6 | |
|---|---|---|---|---|
| | Serum titer | Brain titer | Serum titer | Brain titer |
| Sample | (pfu/mL) | (pfu/brain) | (pfu/mL) | (pfu/brain) |
| XBY-6 #1 | 30,000 | —* | — | — |
| XBY-6 #2 | — | — | — | — |
| XBY-6 #3 | 700 | — | — | — |
| XBY-S2 #1 | 100 | — | — | — |
| XBY-S2 #2 | — | — | — | — |
| XBY-S2 #3 | — | — | — | — |
| Lipo #1 | 2,000 | — | — | 500,000 |
| Lipo #2 | 2,500 | — | — | 6,500,000 |
| Lipo #3 | 15,000 | — | — | 3,500 |
| PBS #1 | 25,000 | — | 100 | 5,500,000 |
| PBS #2 | 20,000 | — | — | 180,000,000 |
| PBS #3 | 4,500 | — | — | 2,500,000 |

*— indicates no virus detected; limits of detection were 50 pfu/mL of serum and 25 pfu/brain

Whether thioaptamers exhibited direct antiviral activity in cell culture was also determined. The direct antiviral activity of the thioaptamer was investigated in cell culture. Using six-well dishes containing Vero cells, duplicate wells were treated with one of the following samples:

| | |
|---|---|
| 1. Liposomes + xbyc2 (10 μg/well) | 2. Liposomes + xbysl (10 μg/well) |
| 3. Liposomes + XBY-S2 (5 μg/well) | 4. Liposomes + XBY-S2 |
| 4. Liposomes only | (10 μg/well) |
| | 5. Buffer only |

Wells were incubated for 12 hours with the samples above and then challenged with WN virus at a multiplicity of infection (MOI) of 0.1. Samples were harvested from each well at 0, 14, 24, 34 and 48 hours. No cytopathic effect was seen until 48 hours post virus infection. Each well was assayed at each time point by hemaggluttination (HA) assay to detect the presence of virus particles. All samples showed no detectable HA (i.e., ≦4 HAU) except for the samples at 48 hours post virus infection when all wells had 32-64 HAUs. These results demonstrate that the thioaptamers have no direct antiviral activity.

One potential explanation for the antiviral activity of thioaptamers is induction of interferon. This hypothesis was investigated by taking groups of four 3-4 week-old female NIH Swiss and treat them with either 10 ug of XBY-S2 in liposomes, liposomes only, or buffer only on day 0 and day 1 post infection, followed by sacrificing mice on day 2 post infection. Serum samples were diluted 1 in 3 and run in ELISAs to detect mouse interferon-α/β, interferon-γ or TNF-α. None of these cytokines was detected in the serum of any of the 12 mice sampled suggesting that interferon was not involved in the antiviral activity induced by thioaptamer XBY-S2.

Tables 5 and 6 demonstrate that the survival of P18 virus infected animals can be prolonged using thioaptamers and thioaptamers can protect the majority of the animals infected with West Nile virus. These results demonstrate that modified thioaptamers alter the outcome of in vivo viral infections by Category A and B agents by the manipulation of transcription factors involved in the immune response. FIG. 1 is a graph that shows survival curves following Pichinde P18 infection in guinea pigs treated with the NF-κB aptamer, XBY-6, the scrambled control, B92, or vehicle, MT, of animals infected by injection of 1000 pfu of Pichinde P18 at day 0, treatment consisted of intraperitoneal injections at days 0, 1 and 2.

FIGS. 2 and 3 are graphs that shows survival curves of guinea pigs with thioaptamers for infection by arenavirus. FIG. 3 is a graph that shows survival curves following West Nile Virus infection in guinea pigs treated with the NF-κB aptamer XBY-6, the AP-1 aptamer XBY-S2, or the liposome vehicle of animals infected by injection with lethal doses of West Nile Virus.

The present inventors have developed thioaptamers that bind specifically to domain III of the envelope protein of flavivurses using the in vitro combinatorial selection methods described herein. Domain III of the envelope protein mediates binding of flaviviruses to host cells (Bhardwaj, et al., 2001, Holbrook and Barrett, unpublished). The epitopes within domain III have been mapped (DW Beasley and AD Barrett, 2002). Using the methods described herein the inventors have also developed thioaptamers to Bio-Safety Level 4 (BSL4) viruses, including the Omsk hermorrhagic fever virus. The partially modified thioaptamers described may be immobilized onto a substrate, e.g., beads or other surfaces, to allow for rapid screening of samples that are suspected of having one or more specific target viruses.

The present invention includes a substrate bound partially thio-modified aptamer, e.g., a bead-bound or array-bound thioaptamers, where the aptamers are selected for high affinity/high selectivity binding to surface protein of a human pathogen, as “bait” to fish out “prey” comprising any epitopically-defined pathogen (virus, bacteria, etc.) or cell (e.g., tumor cell), as described in detail hereinbelow. In conjunction with the identification of target protein specific thioaptamers, detecting, screening, concentration and/or isolation of pathogens that are bound to a specific thioaptamer may be performed using, e.g., flow cytometric sorting to isolate bound pathogens or cells. For example, the purpose of detecting the interaction between the specific thioaptamer and the target protein on a pathogen or pathogen infected cell (or even a transformed cell) may be the presence or absence of the pathogen in a biological or environmental sample. The present invention may also be used to concentrate and/or remove a pathogen from a biological or environmental sample (e.g., water supply, air supply). Key pathogens will be bioterror agents such as anthrax, smallpox, yersinia pestis, tularemia, botulinum toxins, hemorrhagic fever viruses.

Examples of target pathogens include, e.g., toxins toxin selected from the group consisting of Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins.

Target viruses for use with the present invention as protein binding targets and/or antigens include, e.g., Hepatitis B virus, Hepatitis C virus, Yellow Fever virus, Dengue Virus, Japanese Encephalitis virus and West Nile virus. Target proteins also include, e.g., one or more proteins from bioterror agents such as Bacillus, Yersinia, Vibrio, Francisella, Brucella, Clostridium, smallpox viruses, hemorrhagic fever viruses, and neuropathologic viruses. Target proteins may also be cell host proteins that the pathogen(s) use, are require for, or are involved with pathogenicity, infection and/or replication of the pathogen. Examples of hemorrhagic fever viruses include viruses from different viral families. Four viral families are generally implicated in hemorrhagic fever infections, including: (1) Arenaviridae (Lassa, Junin, Machupo, Guanarito, and Sabia viruses, which are the causative agents of Lassa fever and Argentine, Bolivian, Venezuelan, and Brazilian hemorrhagic fevers, respectively); (2) Filoviridae (Ebola and Marburg); (3) Flaviviridae (yellow fever, Omsk hemorrhagic fever, and Kyasanur Forest disease viruses); (4) Bunyaviridae (Rift Valley fever (RFV), Congo-Crimean hemorrhagic fever, and Hemorrhagic fever with renal syndrome viruses. Another virus family for targeting includes Hantaviruses. Target neuropathologic viruses, such as arboviruses and flaviviruses, may also be target antigens. Examples of neuropathologic viruses include, e.g., St. Louis encephalitis, Western equine encephalitis, Eastern equine encephalitis, California encephalitis serogroup (e.g., LaCrosse, Jamestown Canyon, Snowshoe Hare, Trivittatus, Keystone, and California encephalitis viruses), Powassan encephalitis, Venezuelan equine virus, Argentine equine encephalitis virus, Cache Valley virus and West Nile virus. Neuropathologic viruses fall into various viral families and are characterized by symptoms that include: fever of variable severity associated with neurologic symptoms ranging from headache to aseptic meningitis or encephalitis, headache, confusion or other alteration of the senses, nausea and vomiting. Signs may include fever, meningismus, cranial nerve palsies, paresis or paralysis, sensory deficits, altered reflexes, convulsions, abnormal movements and coma of varying degree.

The thioaptamers of the present invention may also be used to target cells for destruction, for example, when the thioaptamer is conjugated with an agent that targets the cell for death. Agents for direct conjugation with a target-specific thioaptamer may include, e.g., a label, a radiolabel, a fluorescent label, a toxin, an aptazyme, an miRNA, an siRNA, an energy-absorbing molecule, a potentiator or even an ionophore. In fact, the agent for cell death may even be a thio-aptamer conjugated Aflatoxin, Botulinum toxin, Clostridium toxin, Conotoxin, Ricin, Saxitoxin, Shiga toxin, Staphylococcus aureus toxin, Tetrodotoxin, Verotoxin, Microcystin (Cyanginosin), Abrin, Cholera toxin, Tetanus toxin, Trichothecene mycotoxin, Modeccin, Volkensin, Viscum Album Lectin 1, Streptococcal toxin, Pseudomonas A toxin, Diphtheria toxin, Listeria monocytogenes toxin, Bacillus anthracis toxic complex, Francisella tularensis toxin, whooping cough pertussis toxin, Yersinia pestis toxic complex, Yersinia enterocolytica enterotoxin, Brucella toxin, and Pasteurella toxins or active subunits thereof.

In come cases the conjugated thioaptamers may also be provided with a potentiator of cell death, e.g., a potentiator such as procodazole, triprolidine, propionic acid, monensin, an anti-sense inhibitor of the RAD51 gene, bromodeoxyuridine, dipyridamole, indomethacin, a monoclonal antibody, an anti-transferrin receptor immunotoxin, metoclopramide, 7-thia-8-oxoguanosine, N-solanesyl-N,N′-bis(3,4-dimethoxybenzyl)ethylenediamine, N-[4[(4-fluorphenyl)sulfonyl]phenyl]acetamide, leucovorin, heparin, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative having potentiator activity, dimethyl sulfoxide and mixtures thereof.

Dosage forms. A dosage unit for use of the aptamers and partially thioaptamers of the present invention, may be a single compound or mixtures thereof with other compounds, e.g., a potentiator. The compounds may be mixed together, form ionic or even covalent bonds. The aptamers and partially thioaptamers of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the aptamers and partially thioaptamers of the present invention to a patient in need of therapy that includes the aptamers and partially thioaptamers. The aptamers and partially thioaptamers may also be administered as any one of known salt forms.

Aptamers and partially thioaptamers is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the aptamers and partially thioaptamers may be formulated to provide, e.g., maximum and/or consistent dosing for the particular form for oral, rectal, topical, intravenous injection or parenteral administration. While the aptamers and partially thioaptamers may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.), and the like, relevant portions incorporated herein by reference.

For example, the aptamers and partially thioaptamers may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol,mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The aptamers and partially thioaptamers may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The aptamers and partially thioaptamers may also be coupled to one or more soluble, biodegradable, bloacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the aptamers and partially thioaptamers may be coupled one or more biodegradable polymers to achieve controlled release of the aptamers and partially thioaptamers, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the aptamers and partially thioaptamers and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions or even for delivery via a suppository. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

Intranasal and Nasal. For direct delivery to the nasal passages, sinuses, mouth, throat, esophagous, tachea, lungs and alveoli, the aptamers and partially thioaptamers may also be delivered as an intranasal form via use of a suitable intranasal vehicle. For dermal and transdermal delivery, the aptamers and partially thioaptamers may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of aptamers and partially thioaptamers may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

Effervescent tablets. To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration. Parenteral and intravenous forms may also include minerals and other materials to make them compatible with the type of injection or delivery system chosen.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

Mini-tabs. For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of pathogenic infection or even a cancer. The kit will generally include one or more containers containing a pharmaceutical composition with a therapeutically effective amount of the aptamers and/or partially thioaptamers disclosed herein. Such kits may further include one or more of various conventional pharmaceutical kit components, e.g., containers with one or more pharmaceutically acceptable diluents, as will be readily apparent to those skilled in the art. Printed instructions, either as inserts or as labels, indicating quantities of the components to be administered, guidelines for mixture and/or administration, may also be included in the kit.

The aptamers and partially thioaptamers and, optionally, one or more potentiators may be mixed with a pharmaceutically acceptable carrier. The carrier may be a solid or liquid and the type is generally chosen based on the type of administration being used. The active agent may be coadministered in the form of a: tablet, capsule, liposome, as an agglomerated powder, in a liquid form or as a suppository.

Combinatorial Selection on Flaviviruses. The family Flavidiridae includes three genera: Flavivirus, Pestivirus and Hepacivirus. These viruses are enveloped and contain a single-stranded, positive sense RNA genome. Viruses among the different genera are related distantly but share conserved nonstructural protein motifs and a similar gene order. Flaviviruses are the causative agents of numerous human diseases and are categorized into, e.g., tick-borne and mosquito-borne viruses (Kuno et al., 1998). The tick-borne encephalitis (TBE) serocomplex includes Omsk hemorrhagic fever (OHF) virus, Central European tick-borne encephalitis (CBE) virus, Russian-Spring Summer encephalitis (RSSE) virus and Kyasanur forest disease (KFD) virus. These pathogenic viruses require bio-safety level 4 (BSL4) handling and have significant biological warfare potential. Langat virus (LGT) is a mammalian tick-borne flavivirus that is nonpathogenic. LGT virus has serological cross-reactivity and genetic homology with the more pathogenic members of the TBE serocomplex. The LGT virus is used as a model system for the BSL-4 TBE pathogens. The mosquito-borne viruses include West Nile (WN) Virus. WN virus is a member of the Japanese encephalitis (JE) serocomplex. Human infections with WN virus may result in potentially fatal encephalitis.

Mature Flavivirus virions are approximately 50 nm in diameter and are composed of three structural proteins including a nucleocapsid protein (C), a small transmembrane protein (M) and a major surface envelope glycoprotein (E) (Chambers, et al., 1990). The M and E proteins form a heteromeric complex on the virion surface (Heinz, et al., 1994). The flavivirus E glycoprotein is the major surface protein and is the primary antigen that induces protective immunity (Crill, et al., Heinz, et al., 1990). Additionally, the E protein mediates receptor binding and is essential for membrane fusion (Rey, et al., 1995; Roehrig, 1997; Roehrig, et al., 1998). The 500 residue E protein contains two transmembrane segments at the carboxy-terminal end (Chambers, et al., 1990). At physiological pH, the E protein forms homodimers on the surface of mature virions (Heinz, et al., 1991). At a pH below 6.5, however, an irreversible conformational change occurs in which the E proteins are rearranged to trimers (Allison et al., 1995). This conformational change is probably necessary for fusion of viral and endosomal membranes following uptake by receptor-mediated endocytosis. The E protein consists of three structural domains. Domain I appears to be the molecular hinge region involved in low-pH-triggered conformational changes (Roehrig, et al., 1998). Domain II, or the dimerization domain, is involved in virus-mediated membrane fusion (Rey, et al., 1995; Roehrig, et al., 1998). Domain III elicits virus-neutralizing antibodies and is an immunoglobulin-like structure that encodes a receptor-binding motif (Rey, et al., 1995; Roehrig, 1997; Roehrig, et al., 1998). Comparisons of E proteins from variant viruses with wild-type viruses have identified several residues in domain III that appear to be involved with receptor recognition (Rey, et al., 1995, Dunster, et al., 1999; Mandl, et al., 2000; Ni, et al., 2000; Ryman, et al., 1998; Lee, et al., 2000; Beasley and Barrett, 2002).

X-ray crystallography of a soluble dimeric fragment isolated from Tick-borne Encephalitis (TBE) virions, referred to as the ectodomain, was used to determine the structure of the flavivirus E protein to 2A resolution (Rey, et al., 1995). TBE is the only flavivirus for which the E protein structure has been elucidated, however, based on sequence homology and the conservation of all six disulfide bridges, the E proteins of all 70 different flaviviruses likely share similar basic architecture.

The E protein of flavivirus exists as a homodimer (Rey, et al., 1995; Heinz, et al., 2001). The E protein subunits are arranged in a head-to-tail orientation. In the mature virion, the E protein lies parallel to the viral membrane. A 50 amino acid long region reaches out from each of the distal ends of the dimer to connect the ectodomain of each subunit. An N-linked carbohydrate side chain is attached to each monomer on the upper surfaces of the ectodomain. Antigenic determinants also exist on these upper surfaces. The three distinct domains (I, II and III) within each of the monomers have mostly β-strand secondary structure and are connected by potential hinge regions (Rey, et al., 1995).

The molecular organization of TBE was investigated by Ferlenghi and coworkers by studying virus-like particles using cryo-EM (Ferlenghi, et al., 2001). The subviral particles were generated by co-expressing the prM and E proteins in mammalian cells. These studies suggest that the E protein dimers form a network of specific lateral interactions in which domain II of one E dimer contacts domains I and III of a bordering dimer. As a result of these interactions, a highly ordered outer shell forms with icosahedral symmetry. The structure was determined to a resolution of 19A. The 30 nm subviral particle is approximately two-thirds the size of the virion and contains 30 copies of the E dimer arranged in an icosahedral lattice with a triangulation number of 1 (T=1). Based on biochemical, functional and immunochemical data, the arrangement of the E dimers in virions is similar to that of the subviral particles (Ferlenghi et al., 2001; Corver et al., 2000; Schalich et al., 1996). When the information from the T=1 subviral particle is extrapolated to a whole virion particle (50 nm), the virion surface appears to be arranged with 90 dimers in a T=3 icosahedral lattice (Ferlenghi et al., 2001).

Domain III Flavivirus Selection—Combinatorial selection methods were used to select single-stranded (ss) DNA aptamers that bind to the envelope domain III regions of Langat Virus, Omsk Hemorrhagic Fever Virus and West Nile Virus. Since domain III is thought to bind to the host cell receptor, aptamers targeting domain III may inhibit viral entry. Monothiophosphate aptamers, or thioaptamers, were selected since the backbone modification renders these agents more resistant to digestion by cellular nucleases.

Libraries and isolation of single-stranded DNA—A library of monothiophosphate oligonucleotides containing a random region of 30 bases flanked by primer regions was converted to double-stranded (ds) DNA in a standard Klenow reaction. The dsDNA template was amplified by PCR in the presence of monothiophosphate-substituted adenosine (αS ATP), CTP, TTP and GTP. The 5′ end of the forward primer contained 3 consecutive biotin labels. The primers were removed from the PCR products using Microcon-YM30 spin columns. The resulting PCR product was attached to Magnetic Porous Glass-Strepatavidin (MPG-Streptavidin, CPG Inc.) beads with gentle rocking for 15 minutes at room temperature. Unbound DNA was washed from the beads. A melting solution of 0.1 M NaOH was added to the beads for 10 minutes at room temperature to denature the dsDNA. A Magnetic Particle Separator (CPG Inc.) was used to separate the biotinylated strand, attached to the magnetic beads, from the unbiotinylated ss DNA library in the supernatant. The ssDNA library was neutralized, and the purity was confirmed by PCR. The pure ssDNA library is amplified by the forward but not the reverse primer.

Combinatorial selection of ssDNA with domain III of Flavivirus E proteins—The selection was performed by incubating the ssDNA library with the domain III protein for 1 hour at room temperature in binding buffer (50 mM Tris base (pH 8.5), 1 mM DTT and 25 mM KCl). Initially, equimolar amounts of the ssDNA library and domain III (approximately 0.5-1.0 μM) were mixed in a total volume of 50 μl. A negative control experiment was also performed without the addition of domain III. Increasing amounts of KCl (up to 250 mM) and decreasing ratios of protein:library were used in the selections to increase the stringency of binding. For the OHFV and LGT domain III selection, the binding mixtures were incubated on a Micropure-EZ centrifugal filter device (Millipore Corp.) for 10 minutes at room temperature and washed with binding buffer. The ssDNA-domain III complex was eluted from the top of the filter with 8M urea. Microcon YM-30 spin columns were used to wash out the urea and concentrated the ssDNA. The ssDNA was amplified by PCR and the resulting library was used in the next round of selection. These iterations are repeated until convergence of the sequences is observed.

Results—The sequences from the Omsk Hemorrhagic Fever and Langat virus domain III selections are shown in Tables 8 and 9, respectively.

TABLE 8

Omsk Hemorrhagic Fever Virus Domain III Sequences

Sequence

| | | |
|---|---|---|
| Initial | | |
| Round | | |
| i-1 | GAG CTG AGT CCC ACC TTT TCT GCG CCG CTC | SEQ ID NO.:30 |
| i-2 | AAC GAA TCG TTT GTG AAC GAC AAA CTT TTC | SEQ ID NO.:31 |
| i-3 | TGC GAC CAC ACC TGT AGT CAT GGC TTA AAT | SEQ ID NO.:32 |
| i-4 | CTA TAT CAG CTA TCA ACA GGA AAA ACT GAG | SEQ ID NO.:33 |
| Round 4 | | |
| 4-6 | GCT GTA TAC CCA TCC ATG CGA TGC CCC TAC | SEQ ID NO.:34 |
| 4-8 | GCC TGT CGT GTG TAT ATG CAC ATT CAC CTG | SEQ ID NO.:35 |
| 4-11 | CGC CCA AAC TTG CAC AGC TAC ACG AAT AGC | SEQ ID NO.:36 |
| 4-12 | AGT TGG CCA CAG CAA AAA CTN CCC TCG CGG | SEQ ID NO.:37 |
| 4-13 | GGC GGT TCT GTC CAT GCC CAT ACT CCC CAC | SEQ ID NO.:38 |
| 4-14 | CCT CCT TCC ACC TCT AGG CGG ATA GAG GGT | SEQ ID NO.:39 |
| 4-15 | GT CTG AAT TTA CGC CTT CTA TCC GCT CCC | SEQ ID NO.:40 |
| 4-16 | ATG TTG CAG CTC CGA TTC AAT GCC GCA CAC | SEQ ID NO.:41 |
| Round 8 | | |
| 8-7 (2)* | GGC CTG TAT ACA CGT GTA CAC CCA CAC AGC | SEQ ID NO.:42 |
| 8-5 (2) | GGC GTG TAC ACC | SEQ ID NO.:43 |
| 8-6 (2) | GC CTG TAC ACC | SEQ ID NO.:44 |
| 8-9 (2) | GGC CTG TAC AC GT GTA CAC C | SEQ ID NO.:45 |
| 8-11 (1) | GGC CTG TAC ACG | SEQ ID NO.:46 |
| Round 10 | | |
| 10-4 (3) | GGC CTG TAC ACG TGT ACA CC | SEQ ID NO.:47 |
| 10-3 (2) | GGC GGC TGT ACA TGT ACA CC | SEQ ID NO.:48 |
| 10-11 (3) | GGC CTG TAC ACC | SEQ ID NO.:49 |
| 10-18 (2) | GGC CCG TAC ACG TGT ACA CC | SEQ ID NO.:50 |
| Round 12 | | |
| 12-2 (7) | GGC CTG TAC ACG TGT ACA CC | SEQ ID NO.:51 |
| 12-13 (1) | GGC CCG TAC ACG TGT ACA CC | SEQ ID NO.:52 |
| 12-11 (1) | GGC CT | |

*Number of clones with identical sequence; the thio-modifications are 5′ to the A's
Notice that the final selected sequence GGC CTG TAC ACG TGT ACA CC SEQ ID NO.:52 is a near perfect palindrome-perhaps forming a duplex or hairpin thioaptamer to the protein.

TABLE 9

Langat Domain III Sequences**

Sequences

| | | |
|---|---|---|
| Round 4 | | |
| 4-2 | ACC GTG CAG ATG GAG TGG GAA TCC AGT ACC | SEQ ID NO.:53 |
| 4-3 | TTG TTG TCC ATA ACG TCG CAA CAC AAG CAC | SEQ ID NO.:54 |
| 4-4 | CGG TTG GGG AGC GCA ATC AAG ATG GGA CGC | SEQ ID NO.:55 |
| 4-5 | GCG TGC ATC TAC ACA TAC CTG CAC TAT GCA | SEQ ID NO.:56 |
| 4-6 | GGA ATG CGN GNG NGN ACN GGA TNN TNC CAC CAN | SEQ ID NO.:57 |
| 4-10 | TGC GCN CGN GGA CCC CCT CCC AAN CTT CAN T | SEQ ID NO.:58 |
| 4-15 | NNN CGN NCN TGG AGG GGT NAA GNG NGA CNC | SEQ ID NO.:59 |
| 4-18 | GTG GTT GAA TCT ACG CGA CAA CTC TGT CCC | SEQ ID NO.:60 |
| 4-19 | GTG AAC ACA AGT TGG ATG TAG GGC GCC AGG | SEQ ID NO.:61 |
| 4-20 | TCC AGC GCA ATA CCT ACC TAG GCA GAT CAC | SEQ ID NO.:62 |
| Round 8 | | |
| 8-18 | TGC CCA CAT GCA GTA GTA CAA TCC CGA CCC | SEQ ID NO.:63 |
| 8-17 | TCC GCA ATT CAG CAT TCC CAT GCA TAC AGG | SEQ ID NO.:64 |
| 8-16 | CTG TAC GCC CAG AGC AGA CAT TAC CCG CAC | SEQ ID NO.:65 |
| 8-15 | GCG TGT ACG TTC AAC CCA AGC ACT GGA CAC | SEQ ID NO.:66 |
| 8-13 | GCC TTC CAC AAC AGC GGA ATG CAC AAG CAC | SEQ ID NO.:67 |
| 8-11 | GGT TGA CAT TGC TGT TCC ATC CAG TGA ACG | SEQ ID NO.:68 |
| 8-9 | CCC ACC GAC GTT TCC TTG GTG TAC CAG CAC | SEQ ID NO.:69 |
| 8-8 | TGG TNT CCC NAC CAC TGN ACA CCT TGA AGC | SEQ ID NO.:70 |
| 8-7 | GGG GCG CGC GTT GAC ACT GTA CAC TGC ACC | SEQ ID NO.:71 |
| 8-6 | CCG CGT CAT ATA GCC TCA TCA AGT GGA CCG | SEQ ID NO.:72 |
| 8-5 | GTG GTA GAC CAC ACT AGA CCA CAT CCT AAC | SEQ ID NO.:73 |
| 8-4 | GCC TGC AGC TCC AGT GTG CAC ACT CAC CAC | SEQ ID NO.:74 |
| 8-3 | NNN NGC AGC ANN NCA ATN CAN NNC CCC CCN | SEQ ID NO.:75 |
| Round 10 | | |
| 10-14 | TCC AGT CTG CAC C | SEQ ID NO.:76 |
| 10-13 | GTG NAN NCN TGG TGN CAN CTC CAA CAC NNN | SEQ ID NO.:77 |
| 10-12 | GCT GTC CAC ACC AC | SEQ ID NO.:78 |
| 10-11 | GTG TGC CTA CNC TGC AAC ACG TG | SEQ ID NO.:79 |
| 10-9 | GCG TGC ACA CTA CAA GTC CAC ACG TCA | SEQ ID NO.:80 |
| 10-7 | GCT GTC CTG TGT ACA CGG TGC AAT GCA AC | SEQ ID NO.:81 |
| 10-6 | GCC GTC CAT ACA GTT TAA CGC GAA CCC AGC | SEQ ID NO.:82 |
| 10-3 | TCG TGT CCA TGC CCG AAC AGA CCA CAA CAG | SEQ ID NO.:83 |
| 10-1 | TGC TGC CAA CAC AAC CAG ACT CCC CAG GCG | SEQ ID NO.:84 |
| Round 11 | | |
| 11-1 | GCG TGT TGT TGT ACA GTC CCC AGT TGT ACC | SEQ ID NO.:85 |
| 11-3 | GCG TGT GCA CAG TCC AAC C | SEQ ID NO.:86 |
| 11-14 | GCG CTG TAT ATC CAT GTG TGG TCC TAA CGC | SEQ ID NO.:87 |
| 11-12 | GTG TAT CGT GTG GTG CAG CC | SEQ ID NO.:88 |
| 11-11 | TGC ATC CAT ACC ACG ATG TAC NNA ACA AC | SEQ ID NO.:89 |
| 11-10 | TGC TCC AAC GTC TCC ATC CAA CGC GAG AGG | SEQ ID NO.:90 |
| 11-9 | GCG TGC CGA CAT TCA CAG TGT TCA NGG AAC | SEQ ID NO.:91 |
| 11-8 | GCT GTA CAC CTG GAC TGG ACA CCA GCA C | SEQ ID NO.:92 |
| Round 13 | | |
| 13-1 | GCG TGT GCA CAG TCC AAC A | SEQ ID NO.:93 |
| 13-3 | GCC TCC ACA CGC ACA G | SEQ ID NO.:94 |
| 13-4 | GCG TGT CCA CGT GCA CAC CCC ACC GC | SEQ ID NO.:95 |
| 13-5 | GCG TGT GCA CAG TCC AAC C | SEQ ID NO.:96 |
| 13-6 | GCC TGC ACA CCT GTA CAC CGC GGC | SEQ ID NO.:97 |
| 13-9 | GCG TGT GCA CAC TGT ACT GTA CAC CCC AGG | SEQ ID NO.:98 |
| 13-8 | TCT ATC CAC GGC | SEQ ID NO.:99 |
| 13-10 | GCC TGT ATA CAC TCC CAG C | SEQ ID NO.:100 |
| 13-11 | GCG TGT GCA CAG TCC AAC C | SEQ ID NO.:101 |
| 13-14 | TGG TGC CCT ACT GCA TGC CGC CCC CAC ACG | SEQ ID NO.:102 |
| 13-15 | CGC TGT GCA CAG TCC AAC C | SEQ ID NO.:103 |
| 13-17 | GCG TGT GCA CAG TCC CCA C | SEQ ID NO.:104 |
| 13-16 | GCG TTG CAT GTG ANC NCC CAG TAC CAC AGG | SEQ ID NO.:105 |
| 13-18 | GCG TGC ACT TGG ACA CCT CCA CAT CGA CCC | SEQ ID NO.:106 |
| 13-19 | NTT GTG CTG GNG CAG NGN ACA TGN ACA CGC | SEQ ID NO.:107 |

**Thio-modifications are 5′ to the A's

Protein Identification. SELDI MS Detection of NF-κB bound to Thioaptamer Surfaces and Beads. The present inventors have demonstrated that thioaptamers bind both purified, recombinant NF-κB p50 and nuclear extracts on either beads (or Ciphergen PBSII ProteinChip surfaces). SELDI MS of p50 binding to various ProteinChips and beads was conducted. Ciphergen's SELDI mass spectrometric methods were used to detect recombinant p50 with using epoxy-activated ProteinChip Arrays. Duplex aptamers with a 5′-amino terminus linked to a 12 carbon chain were synthesized. These duplex aptamers were the dithioate 14-mers XBY-6 (C12-XBY-6), the normal phosphate backbone 22-mer NF-κB binding site with the C12 5′-amino linker (C12-IgκB) or a non-specific, non-covalently linked duplex (polydIdC) as a control. These aptamers were spotted individually onto spots of a preactivated ProteinChip Array (PS20) in 2 μl of 25 mM NaHCO₃ (pH 9) and incubated overnight at room temperature and high humidity. Following incubation, excess aptamer was removed by washing 2 times in 5 μl 25 mM PBS, 0.1% Triton X-100 (pH 7.2) and the surface was blocked to limit non-specific binding with 1 μl of 100 μM bovine serum albumin for 4 hrs. After blocking, excess BSA was washed away as above. Next, 4.3 pmol recombinant p50 was spiked into 100 pmol BSA in 5 μl of optimized EMSA buffer containing 20 mM DTT, 0.01 μM polydIdC and incubated on each of the aptamer/thioaptamer surfaces for 2 hrs at room temperature and high humidity. Following incubation, each spot was washed with 5 μl of 50 mM Tris buffer (pH 7.2), 0.1% CHAPS, 1 M urea, 0.5 M NaCl, followed by a water wash to remove all non-specific binding components. 0.8 μl Sinapinic acid (saturated solution in 50% acetonitrile, 0.5% trifluoroacetic acid) was added to each spot, dried and the array analyzed in the mass reader. The p50 (MW˜46,200) on either the XBY-6 or IgκB bound surfaces was detected, but not the control (data not shown). In other spectra with more stringent washing, the XBY-6 spot, but not the IgκB spot, was shown to retain the bound p50 (spectra not shown), confirming the tighter binding of p50 to XBY-6 (sub-nM) relative to IgκB (KD 4 nM).

The XBY-6 thioaptamer can also capture recombinant p50 (MW˜46,200) on gel beads to which the 5′amino-C12 linked XBY-6 is coupled to 20 ul (1:1) AminoLink® Plus Coupling gel (Pierce, Immunoprecipitation kit, cat # 45335). In this study, 3 μg of C12-XBY-6 was coupled overnight at 4° C. following the kit protocol. After quenching the gel, 6 μg of p50 in 1× EMSA buffer with polydIdC was added to the gel and incubated for 2 hrs with shaking at room temperature. The gel was washed to remove nonspecifically bound proteins, followed by one quick rinse with water. Protein bound to the gel was extracted with 5 μl of organic solvent (50% AcN and 0.01% TFA) with shaking for 20 min. All of the extracts were spotted onto NP20 ProteinChips, dried, followed by addition of saturated SPA and read on the Ciphergen PBSII MS. After extraction, 1 μl of the gel was loaded onto NP20 chip (data not shown). Proteins still bound to the gel was analyzed using saturated SPA on the PBSII. Once again it was found that p50 can be identified by SELDI, both in the extract and retained directly on the beads.

Nuclear extracts were captured onto Ciphergen's PS20 ProteinChip Arrays: either 0.5 μg of C12-XBY-6, 0.25 pm of C12-IgκB or 0.5 μg of poly dIdC were incubated on PS20 chip overnight. The chips were blocked with 7 mg/ml BSA in PBS/0.1% Tween-20. Following blocking, 49 μg of nuclear extract in optimized EMSA buffer were incubated on each spot for 2 hr with shaking. Each spot was washed with PBS/0.1% Triton three times, followed by one quick wash with water. Proteins bound on each spot were analyzed using saturated SPA on the PBSII. These results indicate that a protein was bound with a MW˜105,591, which may represent p105, the precursor to p50 or the p50/p50 homodimer.

Bead-based phosphorodithioate and phosphorothioate thioaptamer combinatorial libraries and high throughput sorting against targeted proteins. The one-bead, one-aptamer split synthesis method disclosed herein was used to identify a specific ODN aptamer that targets proteins or other biomolecules. In combination with the split and pool synthesis combinatorial chemistry method for creating a combinatorial library of oligonucleotide agents (either phosphate, monothiophosphate or dithiophosphate); both monothiophosphate and dithiophosphate combinatorial libraries attached to individual support beads were shown to produce aptamers that demonstrate target-specific binding. Proteins that bind tightly to only a few of the 10 4108 different support beads may be selected by binding either purified proteins, nuclear or cytoplasmic extracts or pools of proteins to the beads and then identifying which beads have bound target protein by immunostaining, fluorescent staining techniques or MS (SELDI). Thus, the methods and compositions created and isolated thereby allow for rapid screening, isolation and identification of specific thioaptamers that bind to proteins such as NF-κB and AP-1 using the PCR-based identification tag of the selected bead disclosed herein.

Preparation and composition of the thioaptamer libraries and libraries of libraries. Depending on the nature of the targeted protein, thioaptamer combinatorial libraries were created that cover appropriate sequence space relative to the targeted protein. For transcription factors duplex thioaptamers were create that have a significant population of sequences similar to the consensus sequence. In the case of the in vitro combinatorial selection approach disclosed herein, the complexity of the library can be as large as 10¹⁴ different sequences and thus can cover all sequence space for a small (<22 nt) duplex. For the bead-based thioaptamer libraries, complexity is limited to the number of different beads—10⁶-10⁸, depending on their size.

To increase the complexity of the libraries one may also use a novel iterative approach in which a bead-based library of libraries of thioaptamers is made in which as many as 10⁶ different thioaptamers are attached to a single bead and thus have a total complexity of as many as 10¹²-10¹⁴ sequences in the library of library. For example, a library of libraries was prepared on a 1 μmole scale of polystyrene beads (60-70 μm). The downstream and upstream primers, 5′-d(GGATCCGGTGGTCTG)-3′ SEQ ID NO.: 108and 5′-d(CCTACTCGCGAATTC)-3′ SEQ ID NO.: 109 were synthesized in parallel on a two-column DNA synthesizer (Expedite 8909, Applied Biosystems). Following the 5′-primer, the sequences programmed on the synthesizer for the combinatorial library were 5′-AT*GN*GA*AT*TT*NC*CA 3′ SEQ ID NO.: 110 on column 1 and 5′-GG*AG*NG*CN*CA*GG*AC-3′ SEQ ID NO.: 111 on column 2. The 3′-primer sequence completed the 44-mer programmed on the synthesizer. A “split and pool” was used at each position indicated by an asterisk in order to synthesize the combinatorial region for the library of libraries. The letter N indicates a mixture of four bases (A, C, G and T). Five of the beads were randomly selected from the library and “one bead one PCR” was run, cloned and sequenced. The results listed below indicated the successful construction of the library of libraries.

| | | |
|---|---|---|
| E45-2-1: | 5′-GG AG GA CT TT CC AC-3′ | SEQ ID NO.:112 |
| E45-2-2: | 5′-GG AG GA CA TT GC AC-3′ | SEQ ID NO.:113 |
| E45-2-4: | 5′-GG AG GA CC TT CC AC-3′ | SEQ ID NO.:114 |
| E45-2-5: | 5′-GG AG GA CC TT GC AC-3′ | SEQ ID NO.:115 |
| E45-2-11: | 5′-GG AG GA CN TT TC AC-3′ | SEQ ID NO.:116 |
| E45-2-12: | 5′-GG AG GA CC TT TC AC-3′ | SEQ ID NO.:117 |
| E45-3-1: | 5′-GG GA TG GT CA GG AC-3′ | SEQ ID NO.:118 |
| E45-3-3: | 5′-GG GC GG AT CA GG AC-3′ | SEQ ID NO.:119 |
| E45-3-5: | 5′-GG GA AG AT CA GG AC-3′ | SEQ ID NO.:120 |
| E45-3-6: | 5′-GG GG TG AT CA GG AC-3′ | SEQ ID NO.:121 |
| E45-3-11: | 5′-GG AG TG CT CA GG CA-3′ | SEQ ID NO.:122 |
| E45-6-1: | 5′-GG AG CG GT GT CC AC-3′ | SEQ ID NO.:123 |
| E45-6-2: | 5′-GG GA GG GA TT AC CA-3′ | SEQ ID NO.:124 |
| E45-6-3: | 5′-GG AG CG GT TT GC CA-3′ | SEQ ID NO.:125 |
| E45-6-10: | 5′-GG AG CG AT TT CC CA-3′ | SEQ ID NO.:126 |
| E45-6-11 | 5′-GG AG AG GT TT TC CA-3′ | SEQ ID NO.:127 |
| E45-7-1: | 5′-AT AG GG CA CA GG AC-3′ | SEQ ID NO.:128 |
| E45-7-2: | 5′-AT AG NG CC CA GG AC-3′ | SEQ ID NO.:129 |
| E45-7-5: | 5′-AT AG GG CG CA GG AC-3′ | SEQ ID NO.:130 |
| E45-8-1: | 5′-GG AG GG CC CA GC AC-3′ | SEQ ID NO.:131 |
| E45-8-2: | 5′-GG AG AG CA CA TC AC-3′ | SEQ ID NO.:132 |
| E45-8-3: | 5′-GG AG CG CG CA CC AC-3′ | SEQ ID NO.:133 |
| E45-8-4: | 5′-GG AG CG CG CA GC AC-3′ | SEQ ID NO.:134 |
| E45-8-5: | 5′-GG AG GG CT CA GC AC-3′ | SEQ ID NO.:135 |
| E45-8-6: | 5′-GG AG AG CA CA AC AC-3′ | SEQ ID NO.:136 |
| E45-8-10: | 5′-GG AG CG CG CA TC AC-3′ | SEQ ID NO.:137 |
| E45-8-11: | 5′-GG AG AG CG CA CC AC-3′ | SEQ ID NO.:138 |

For proteins in which there are no known sequence to design the library, the user of the present invention begins with a single-strand (ss) DNA or RNA thioaptamers with at least 30 nts in the randomized or combinatorial regions. Using the methodology created and developed by the present inventors for creating both duplex and ss DNA and RNA thioaptamer libraries by both enzymatic and bead-based methods. One such technique is the one-bead, one-ODN library ligation reaction in which short (15 nucleotides) 5′- and 3′-sequences are sufficient to serve as primers for bead-based PCR (Yang, et al., 2002). To achieve even longer combinatorial segments, it is possible to eliminate entirely one of the primer segments. High quality one-bead one-oligo libraries were contructed by join two pieces of DNA based on an enzymatic ligation reaction or using highly active phosphorothioate towards 5′-iodo groups on the ODN. Standard phosphoramidite chemistry was used for synthesis of 5′ monophosphate ODN (5′-P(o)CCAGGAGATTCCAC-GGATCCGGTGGTCTGT-bead) SEQ ID NO.: 139. The fully protected ODN with the non-cleavable linker beads were treated with concentrated ammonia at 37° C. for 21 hours to remove the protecting groups while allowing the ODN to remain attached to the beads. A selected single bead was mixed with the following components: 3 μl of 40 μM 15 mer oligonucleotide (5′-CCTACTCGCGAATTC-3′, 3 μl of 10× ligation buffer, 3 μl of DMSO, 2 μl of T4 RNA ligase and 19 μl of ddH₂O. The reaction was performed at 5° C. for 17 hrs. The supernatant was removed carefully and washed with water. The single bead PCR reaction was run under established conditions. The PCR products were analyzed on a 15% native polyacrylamide gel. The PCR product was cloned using the TA Cloning procedure (Invitrogen) and sequenced on an ABI Prism 310 Genetic Analyzer (Applied Biosystems). The desired sequence (5′-CCTACTCGCGAATTC-P(o)CCAGGAGATTCCAC-GGATCCGGTGGTCTGT-bead) SEQ ID NO.: 140 was obtained.

These results show that the additional nucleic acid sequences may be added to the one-bead, one-ODN library with high quality and efficiency while maintaining the integrity of the library. The ligation reaction allows longer random regions of aptamers to be synthesized on the beads with higher yield since a primer region does not have to be stepwise synthesized onto the bead sequence. The beads were screened for the ability to bind the appropriate protein (such as the various NF-κB dimers or AP1 dimers) labeled with the Alexa Fluor 488 dye (Molecular Probes) or by binding fluorophor labeled antibodies as previously described. After thoroughly washing the protein-bound beads with PBS and 0.1% Tween 20 to minimize nonspecific binding, the beads are sorted using a multicolor flow cytometry and cell/bead sorting to visualize and sort the protein-bound thioaptamer beads and select the tightest binding thioaptamer-protein complexes. The most intensely stained beads will be retrieved. Initially, the inventors concentrated on the NFκB and AP-I dimers, but these methods may be applied to other proteins involved in the immune response. Multicolor flow cytometry was capable of sorting at speeds of 10⁸ beads per hour or viewed in terms of assays for thioaptamers binding to target proteins, 10⁸ assays per hour.

High throughput sorting (HTS) of homo- and heterodimers to thioaptamers by multi-color flow cytometry using multi-color flow cytometry HTS may be used to select thioaptamers that bind preferentially to heterodimers of proteins. As described above, one monomer is tagged fluorescently with a dye (cy3 for example) and a different monomer with another dye (cy5 for example). Both proteins are mixed together and allowed to bind to the bead thioaptamer library. Next, two-color flow cytometry is used to compare cy3/cy5 color levels of each bead. To select homodimers that have high affinity for homodimer A.A, beads that have high cy3 levels and low cy5 levels are selected. Conversely, high cy5/low cy3 indicates a thioaptamer sequence with selectivity for the B.B dimer. For heterodimers, beads are selected for cy3/cy5 levels close to 1. SELDI MS may be used to determine which proteins have been bound to selected combinatory thioaptamer beads and also used with single bead PCR to identify which bead(s) in the combinatorial library have bound to protein(s).

More than 2 dyes and multi-color flow cytometry may be used to select various multimers. Thus, for NF-κB, at least 3 of the 5 different monomeric forms of the protein are combined, each with a different fluorphor and use 3-color flow cytometry to select thioaptamers that have high affinity and selectivity to homodimers A.A, B.B, C.C and various heterodimeric forms from the libraries. In principle, there are few limits to the number of detectable markers (e.g., fluorochromes) that may be used with the present invention, e.g., 5-color flow cytometry may be used.

Sequencing may also be performed directly on the bead. Each individually selected bead is washed thoroughly with 8 M urea (pH 7.2) to remove the protein and directly used for “one-bead one-PCR” amplification using the 5′ and 3′ end primers (Yang, et al., 2002). The PCR products are TA cloned and sequenced as previously described to create hybrid thioaptamers with normal phosphate, monothiophosphate, and dithiophosphate mixed backbones as well, keeping the total thiophosphate backbone below 80% to minimize “non-specific” sticking.

The current approach demonstrated in the above examples requires a different nucleotide sequence to identify a backbone modification. Thioaptamer libraries were also created that only differ in the position of phosphate or dithioate but not in its base sequence. It has been shown that the positions of thiophosphates in a mixed backbone S—ODN can be determined by reaction of the S—ODN with iodoethanol followed by base catalyzed cleavage of the thiophosphate triester. This approach was used to identifying the location of monothio- and dithiophosphate linkages, independent of base sequence.

Massively parallel, thioaptamer bead-based High Throughput Screen of the host and pathogen proteome may be used with the thioselection technology (both enzymatic [S]-ODN and synthetic [S]—ODN/[S₂]—ODN) to develop thioaptamers targeting very important proteins (e.g., NF-κB and AP-1) to identify promising therapeutic leads. Up to 1000's of different proteins in human and pathogen proteomes by using a massively parallel, thioaptamer bead-based HTS of the proteomes with specialized high-throughput multicolor flow cytometry/bead sorting in conjunction with SELDI™ mass-spectrometric methods to identify potential new therapeutic targets both of proteins involved in the immune response to BT viruses as well as viral proteins (Gorenstein et al., patent submitted, 2002). Thioaptamers may be identified to inhibit the differentially expressed proteins in host-pathogen interactions as well as underlying immune response processes and so ameliorate cytopathological immune responses resulting in shock or to enhance “innate immunity” to help mount a more effective immune response.

Mass spectrometric protein detection technology can be used to identify bound proteins using HTS of thioaptamer beads. This approach has significant advantages, since MS is more sensitive than fluorescent imaging and will be very useful for low-abundance proteins. In addition, if more than one protein binds to a given thioaptamer bead, then it will be possible to identify and quantify these proteins by SELDI. This is particularly helpful for identifying non-covalent dimers such as NF-κB or AP-1 (there are 22 different monomeric forms of AP-1 and thus in principle 100's of different combinations of dimers possible).

Thioaptamer proteomic arrays were used to demonstrate the use of ProteinChip array technology (e.g., Ciphergen) for protein identification of modified thioaptamer beads or surfaces. SELDI MS combines the well-established principles of solid-phase extraction and time-of-flight mass spectrometry in a process known as surface enhanced laser desorption/ionization time-of-flight mass spectrometry. ProteinChip Arrays may be customized by covalently attaching affinity reagents such as the modified thioaptamers to the spot surface. If the biological marker to be detected is known and thioaptamer affinity reagents are available, affinity surfaces can be designed to make use of this specific thioaptamer-protein interaction. Also, because SELDI uses mass spectrometric detection, several assays can be multiplexed easily by taking advantage of the unique masses of each bound protein.

High-throughput screening (HTS) of thioaptamer libraries by flow cytometry and SELDI. Bead-based methods were used to identify both thioaptamer sequences and binding proteins in parallel, without the need to select one thioaptamer for each purified protein. A number of [S]—ODN or [S₂]—ODN combinatorial libraries are synthesized, each containing 10⁶ to 10⁹ different, but related members (or a library of library with up to 10¹⁴ sequences). The solid-phase split synthesis described herein may be used to create thioaptamer-bound bead libraries (one bead, one sequence or one library) as above. Each library can be sufficiently different to provide high affinity and selectivity to a small number of cellular proteins (such as AP-1 or NF-κB-type sequences). One or more of the thioaptamer library beads are incubated with cellular extracts, washed thoroughly to remove weakly bound proteins and the bound proteins visualized by direct fluorescent staining with cy3, cy5, SYPRO Ruby, or other newer dyes for high sensitivity (sub-nanogram). Fluorescently stained beads can be sorted in the high-speed cellibead sorter for the top 10² or more beads which have the highest amount of bound protein. The beads selected with the greatest amount of protein bound will then be analyzed by SELDI MALDI-TOF mass spectrometric techniques determine which proteins are bound to each bead; even if more than one protein binds to the bead, the thioaptamer may be used to identify a select group of proteins in cell extracts. The beads selected are then analyzed by SELDI methods to identify if a fairly limited number of different proteins are bound to the specific bead. Alternatively, proteolysis of the proteins on the bead with trypsin and analysis of the peptide fragments by LC MS/MS QTOF2 can be used to identify the proteins on each bead. After removal of protein from the beads by detergent and urea, the thioaptamer sequence on the bead can be determined by the PCR “one bead sequencing” method disclosed herein. Thus, a random library of “sticky beads” is selected and an extract containing the complete proteome to identify both the thioaptamer sequence on the single beads and the protein(s) bound.

HTS of combinatorial libraries to protein mixtures. Besides using cell extracts, known mixtures of hundreds of commercially available proteins (cytokines, transcription factors, etc.) may be applied to the mixture of thioaptamer bead libraries. HTS cell/bead sorting is used followed by MS identification of bound proteins. This involves direct SELDI determination of the protein or peptide fragmentation methods followed by MS identification of bound proteins. A major advantage in using thioaptamers rather than beads with proteins or monoclonal antibodies attached to them is that proteolysis and MS peptide identification is not complicated by proteolysis of bait proteins or Mab's. This approach can be used in parallel with other commercially available antibodies for virtually any protein (particularly AP-1), and serves as an alternative to the more general screening of the complete proteome and identification by SELDI MS methods alone. Once the sequences of the thioaptamers are identified, these are synthesized in larger quantities as reagents for diagnostics and therapeutics.

HTS of Thioaptamers Targeting Differentially Expressed Proteins in the Proteome in virus infected cells. The thioaptamer-based multi-color flow cytometry HTS may also be used for targeting differentially expressed proteins within the host and pathogen proteomes, combined with MS detection (SELDI). The thioaptamer bead-based combinatorial library can be used in conjunction with fluorescent tagging of proteins followed by SELDI MS to identify proteins differentially expressed in control vs. virus infected cells. In this simple two-color assay, a combinatorial library (or a combinatorial library of libraries) of thioaptamer beads may be synthesized, each bead with a single thioaptamer sequence (or a combinatorial library of thioaptamer sequences on each bead). Up to 10⁸ beads can be created with a single thioaptamer sequence on each bead. Cell extracts of a sample such as uninfected cells is labeled fluorescently with a dye (cy3 for example) as carried out previously and a virus-infected cell extract is then labeled fluorescently with another dye (cy5 for example). Both cell extracts are mixed together and allowed to bind to the bead thioaptamer library. Next, two-color flow cytometry is used to compare cy3/cy5 color levels of each bead. If cy3/cy5 level differs significantly (>2-fold) from 1, then the be was captured. To determine which protein(s) have been bound to selected thioaptamer bead, SELDI MS will be used to characterize the bound target further. SELDI MS can be used to determine which proteins have been bound to selected combinatory thioaptamer libraries and also used with single bead PCR to identify which bead(s) in the combinatorial library have bound to protein(s). As shown above, Ciphergen's ProteinChip epoxy modified surfaces may be used to covalently attach 5′-amino-linker thioaptamers to beads. Ciphergen's ProteinChip array technology allows for solid-phase extraction to desorb more weakly bound proteins to thioaptamer surfaces, followed by surface enhanced laser desorption/ionization time-of-flight mass spectrometry (SELDI-MS). Other diseases besides viral infections may be similarly targeted.

HTS of thioaptamers targeting differentially expressed proteins in the proteome in virus infected cells relative to treated cells (“High Throughput Pharmacoproteomics”). In this embodiment, three-color thioaptamer library bead sorting is used. In this three-color assay, a combinatorial library (or a combinatorial library of libraries) of thioaptamer beads is synthesized, each bead with a single thioaptamer sequence (or a combinatorial library of thioaptamer sequences on each bead). Up to 10⁸ beads with a single thioaptamer sequence on each bead (or 10¹⁴ sequences on the library of libraries) are made. Uninfected cell extracts (or control extracts) are labeled fluorescently with a cy3 for example. A virus-infected cell extract (or any disease cell extract such as cancerous cells) is labeled fluorescently with cy5, and then a thioaptamer therapeutic treated, virus infected (or other disease) cell culture is labeled with a third dye. The three proteome cell extracts are mixed together in equal total protein quantities and allowed to bind to the bead thioaptamer library (or library of libraries). Three-color flow cytometry is used to compare cy3/cy5/dye 3 color levels of each bead. If cy3/cy5 level differs from 1 (uninfected vs. infected) and cy5/Sypro Ruby differs from 1 (infected vs. infected and treated) differs from 1, then the bead can be captured. Such a control assures that the thioaptamer drug previously identified as a promising lead does affect specific protein levels. To determine which protein(s) have been bound to selected thioaptamer beads, SELDI MS can be used to characterize the proteins bound to the target bead.

In one embodiment of the invention a complex of combinatorial libraries are created in which multiple transcription factor-like sequences with varying thiophosphate substitution patterns are concatenated in a single long sequence so that it can bind to multiple transcription factors such as NF-κB, AP-1, SP-1, GRE, SRE, etc., requiring a thioaptamer sequence of at least 20-40-mers. These embodiments provide an attractive approach to defining therapeutic strategies in which multiple proteins can be targeted with multiple thioaptamers. Such a combination (adjuvant) of drug therapeutics is needed to improve immune responses in cancer, AIDS, etc. Mammalian protein signaling pathways are often redundant so that if one pathway is affected, another can take over control. By perturbing multiple, highly interwoven pathways, a greater opportunity to modulate the immune response network is made available.

HT flow cytometry and bead selection. High-throughput screening (HTS) of thioaptamer beads using high-speed multicolor flow cytometry/cell sorting is used. In principle, more than 10¹⁰ beads could be screened within a single day, and specific bead subpopulations could be sorted for subsequent proteomics analysis. This group also has considerable experience in HTS of cells and bacteria (as well as beads) for subsequent molecular characterizations by PCR and gene expression microarray analysis.

Advanced HTS technologies may be used for large library screening and functional genomics. Single-cell (or bead) sorting of rare subpopulations may be used to isolate single beads from combinatorial libraries. A special high speed sorter uses a unique two-stage signal processing system, configured in hardware as a single layer neural network, which allows for sophisticated cell or bead classifications based on multivariate statistics or learning through neural networks.

A 6-color high-speed flow cytometer/cell sorter is configured in hardware and software as a single-layer neural network that can also be used to generate real-time sort decisions on the basis of multivariate statistical classification functions. While it can perform the usual two-way sorts it is commonly used in “straight-ahead” sorting mode to allow for extremely high sort recovery and purity at high throughput rates or to efficiently sort single cells for cloning or for subsequent molecular characterizations by PCR.

Multi-color flow cytometry as a quantitation and validation tool for proteomics. These capabilities can also be used to sort for thioaptamers that bind heterodimers or more complex protein mixtures. By using different fluorescently labeled dyes bound to specific proteins, beads are sorted simultaneously that bind homodimers and heterodimers. A covalently labeled p50 with Alexa-Fluor 488 dye was isolated (data not shown) and carried out 1- and 2-color thioaptamer bead sorting.

Production of large quantities of hybrid dithiophosphate aptamer. Using chemistry developed independently in both Caruthers′ and Gorenstein's laboratory, the most promising dithioate hybrid backbone aptamers show good in vitro and in vivo binding to the targets will be synthesized (Cho, et al., 1993; Farschtschi & Gorenstein, 1988; Gorenstein, et al., 1990; Gorenstein, et al., 1992; Piotto, et al., 1991) on a 5-10 μmole scale and purified (Mono Q; Yang et al., 1999; 2002).

Preparation of nuclear and cytoplasmic extracts was conducted at various times after virus infection, and parallel uninfected control cultures of 5×10⁷ cells are harvested and collected by centrifugation. Cell pellets are resuspended and washed in phosphate buffered saline (PBS). Next, cells are lysed and the cytoplasmic and nuclear fractions isolated. The nuclei are purified by centrifugation through a cushion of 2M sucrose before protein extraction. The protein content in all fractions will be determined by BCA Assay according the manufacturer's directions (Pierce, Rockford, Ill.).

Mass spectrometric identification of bound proteins. As demonstrated above, sorted “positive” beads can be subjected to SELDI-MS analysis to confirm the identity of the proteins bound to the thioaptamer beads of the present invention (via MALDI MS molecular ion characterization). In cases where the “positive” bead's thioaptamer might have bound not only the target protein but other proteins in a sample, e.g., a secondary or even tertiary, etc. protein, SELDI-MS may be used to identify this event through the detection of multiple molecular ions.

Liquid Chromatography/Tandem Mass Spectrometry (LC/MS/MS). For proteins which cannot be identified from the MI, proteolysis and multidimensional LC applying 2D chromatographic separation of peptides is used on-line with MS analysis (Link et al., 1999; Washburn et al., 2001). This LC tandem MS approach is carried out using strong cation exchange (SCX) chromatography combined with reversed-phase (RP) chromatography. Using a salt step gradient, tryptic peptides of complexes are eluted from the SCX column onto the RP column, and contaminants of salts and buffers are washed to waste using a diverter valve. Peptides are subsequently eluted from the RF column directly into the MS, either for mass fingerprinting, or for MS/MS sequence analysis. This LC tandem MS procedure is very useful for small amounts (femtomol) of complex. Yet another procedure is tandem LC/tandem MS. The proteomes can be either human, GP, hampster or mouse-human and mouse genome databases are available.

LC or 2D SDS-PAGE and MS. These techniques are currently the major analytical tools used to identify proteins in the proteome. Thioaptamer bead libraries may be used to differentially screen the proteomes, using 2D gel analysis for differential analysis of protein expression. To improve the comparative analysis of gel imaging imaging software may be used to improve result resolution, e.g., using Nonlinear USA, Inc. (Progenesis). The automated imaging features of this 2D imaging software reduce gel evaluation times substantially and are an important step towards hands-free analysis.

2D gel electrophoresis. 2D PAGE can be conducted essentially as first described by (O'Farrell, 1975). High-throughput may be employed Pharmacia's IPGphor multiple sample IEF device or the first dimension, and Biorad's multiple gel SDS-PAGE systems (Protean Plus and Criterion dodeca cells) for the second. Gels will be stained with either SYPRO Ruby for high sensitivity (sub-nanogram) or Coomassie Blue when less sensitivity is required. Image analysis of gels will be achieved with a Perkin Elmer (PE) ProEXPRESS Proteomic Imaging System using Nonlinear's Progenesis imaging software. A Genomic Solutions' robotics recently purchased is utilized for protein spot picking and for sample trypsin hydrolysis (Proteomic Protein Picker), and sample clean-up, and sample application to MALDI plates (ProPrep 4 Block System). Mass fingerprinting for protein identification may use an Applied Biosystems (AB) matrix-assisted laser desorption/ionization (MALDI) time-of-flight (TOF) Voyager DE STR MS. Proteins will be identified with the Voyager's Prospector software. De novo sequencing and analysis of posttranslational modifications can be achieved by electrospray (ESI) MS/MS (capillary LC nanoflow option).

Isotope-coded affinity tags (ICAT). Some differential protein expression use isotope-coded affinity tags (ICATs) for quantitative analysis of complex protein mixtures (Gygi, et al., 1999). In this procedure, there is an option to fractionate proteins before to proteolysis decreases the complexity of proteins analyzed.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method for isolating pathogen-specific aptamers, comprising the steps of:
contacting a pathogen sample comprising a target with an aptamer library; and
determining the sequence of the one or more aptamers that bind specifically to the target in the pathogen sample.

2. The method of claim 1, wherein the aptamer library is immobilized on a substrate.

3. The method of claim 1, wherein the aptamer library comprises one or more thio-modified aptamers.

4. The method of claim 1, wherein the aptamer library comprises one or more partially thio-modified aptamers.

5. The method of claim 2, wherein the substrate comprises one or more beads.

6. The method of claim 2, wherein the substrate comprises a chip.

7. The method of claim 1, wherein the aptamer library comprises a one-bead, one aptamer library.

8. The method of claim 1, wherein the aptamer library comprises a library of bead libraries.

9. The method of claim 1, wherein the pathogen sample is a selected from the group consisting of viruses, prokaryotes and eukaryotes.

10. The method of claim 9, wherein the prokaryote is a bioterror bacterium.

11. The method of claim 1, wherein the aptamer binds to a surface molecule of the pathogen.

12. The method of claim 10, wherein the surface molecule comprises a cell surface protein, a lipid, a carbohydrate, an envelope protein or an envelope glycoprotein.

13. The method of claim 1, wherein the sample comprises a biological fluid, an aqueous solution, an organic solution, or an air sample.

14. The method of claim 1, wherein the sample comprises one or more environmental samples.

15. The method of claim 1, further comprising the step of performing a surface enhanced laser desorption ionization mass spectroscopy analysis of the sample.

16. The method of claim 1, further comprising the step of performing a SELDI/MALDI-TOF analysis of the sample.

17. The method of claim 1, further comprising the step of isolating the one or more beads that bind specifically to the pathogen using flow cytometry.

18. The method of claim 1, wherein the pathogens are bioterror agents.

19. The method of claim 1, wherein the pathogen sample comprises a bioterror agent selected from the group consisting of Bacillus, Yersinia, Francisella, Vibrio, Brucella, Clostridium.

20. The method of claim 1, wherein the pathogen sample comprises a bioterror agent selected from the group consisting of smallpox viruses, hemorrhagic fever viruses, and neuropathologic viruses.

21. The method of claim 1, wherein the pathogen sample comprises a toxin selected from the group consisting of Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins, mixtures thereof or active subunits thereof.

22. The method of claim 1, wherein the pathogen sample comprises a Biosafety Level 4 pathogen.

23. The method of claim 1, wherein the method is adapted for high-throughput screening by automating the step of contacting one or more samples to one or more aptamers.

24. The method of claim 1, wherein the method is adapted for remote screening of one or more pathogens.

25. The method of claim 1, wherein the aptamer library comprises an S—ODN aptamer library.

26. The method of claim 1, wherein the aptamer library comprises an S₂—ODN aptamer library.

27. The method of claim 1, wherein the aptamer library is attached to a one or more beads by a hexaethyleneglycol linker.

28. The method of claim 1, wherein the aptamer library comprises a nuclear transcription factor protein binding site sequence.

29. The method of claim 1, wherein the aptamers are isosteric and isopolar.

30. The method of claim 1, wherein the aptamers are achiral.

31. The method of claim 1, wherein each of the aptamers are the same length.

32. The method of claim 1, wherein each of the aptamers further comprises a detectable marker.

33. The method of claim 1, wherein the pathogen further comprises a detectable marker.

34. The method of claim 1, wherein the aptamers further comprise one or more fluorophors attached to the 5′ end, the 3′ end or internally within the aptamers.

35. The method of claim 1, wherein the aptamer further comprise the complementary strand to the aptamer.

36. The method of claim 1, wherein the library is double-stranded.

37. The method of claim 1, wherein the library is a peptide nucleic acid library.

38. The method of claim 1, wherein the aptamer comprises both modified and unmodified nucleotides.

39. The method of claim 1, wherein the aptamer comprises both modified and unmodified nucleotides and the aptamer is partially thio-phosphate modified.

40. The method of claim 1, wherein the complementary strand of the modified nucleotide aptamer is selected from the group consisting of dATP(αS), dTTP(αS), dCTP(αS), dGTP(αS).

41. The method of claim 1, wherein the aptamer is selected by amplifying an aptamer library enzymatically using a mix of four nucleotides, wherein at least a portion of at least one and no more than three of the nucleotides in the mix is thiophosphate-modified, to form a partially thiophosphate-modified oligonucleotide combinatorial library.

42. The method of claim 1, wherein no more than three adjacent phosphate sites of an individual aptamer in the aptamer library are replaced with phosphorothioate groups.

43. The method of claim 1, wherein the aptamer library is created by a split and pool combinatorial synthesis chemistry.

44. The method of claim 1, wherein the aptamers are selected from the group consisting of the isolated nucleic acids having SEQ ID NOS. 30 through 107 and combinations thereof.

45. A aptamer library for use in the method of claim 1.

46. A pathogen-specific aptamer comprising a partially thio-modified aptamer that specifically binds to a pathogen.

47. The aptamer of claim 46, wherein the pathogen-specific aptamer is selected from SEQ ID

48. The aptamer of claim 46, wherein the pathogens are bioterror agents.

49. The aptamer of claim 46, wherein the pathogen comprises a bioterror agent selected from the group consisting of Bacillus, Yersinia, Francisella, Vibrio, Brucella, Clostridium.

50. The aptamer of claim 46, wherein the pathogen comprises a bioterror agent selected from the group consisting of smallpox viruses, hemorrhagic fever viruses, and neuropathologic viruses.

51. The aptamer of claim 46, wherein the pathogen comprises a toxin selected from the group consisting of Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins, mixtures thereof or active subunits thereof.

52. The aptamer of claim 46, wherein the pathogen comprises a Biosafety Level 4 pathogen.

53. The aptamer of claim 46, wherein the pathogen comprises a flavivirus.

54. The aptamer of claim 53, wherein the flavivirus pathogen comprises Hepatitis C virus, Yellow Fever virus, Dengue Virus, Japanese Encephalitis virus, West Nile virus, St. Louis encephalitis virus, Murray Valley encephalitis, tick-borne encephalitis, Omsk hemorrhagic fever virus and Langat virus.

55. The aptamer of claim 46, wherein the pathogen comprises a hepadnavirus.

56. The aptamer of claim 46, wherein the pathogen comprises a coronavirus.

57. A method for isolating a pathogen comprising the steps of:
contacting a library of thioaptamer beads with a labeled pathogen sample; and
sorting the thioaptamer-beads that bind specifically to the labeled pathogen by flow cytometry.

58. The method of claim 57, wherein the library of thioaptamer beads is further defined as comprising a one-bead, one-aptamer library.

59. The method of claim 57, wherein the library of thioaptamer beads is further defined as a library of libraries library.

60. The method of claim 59, wherein the library of thioaptamer beads is further defined as a library of libraries library comprising between about 100 to about 10¹² beads with each bead comprising about 10⁵ different thioaptamers.

61. The method of claim 57, further comprising the step of labeling the pathogen sample with a detectable marker specific to a known surface protein of the pathogen.

62. The method of claim 57, further comprising the step of washing uncomplexed sample material off the thioaptamer beads to improve the signal-to-noise ratio prior to the sorting step.

63. A method for identifying thioaptamers that bind to a target protein comprising the steps of:
deproteinating with urea to remove bound protein from one or more thioaptamer beads that have bound specifically to the target protein; and
sequencing the aptamer to obtain the sequence of the aptamer on the one or more beads that bound the target protein.

64. A method for identifying thioaptamers that bind to a target protein comprising the steps of:
(a) deproteinating with urea to remove bound protein from one or more thioaptamer beads that have bound specifically to the target protein,
(b) subjecting the deproteinated beads to PCR to produce a PCR product; and
(c) sequencing the PCR product to obtain the sequence of the aptamer on the one or more beads that bound the target protein.

65. The method of claim 64, wherein the target protein comprises a toxin.

66. The method of claim 64 wherein the target protein comprises a portion of a virus, a viral surface protein, a viral envelope protein, a viral glycoprotein, a viral structural protein or portions thereof.

67. The method of claim 64, wherein the target protein comprises an isolated protein.

68. The method of claim 64, wherein the target protein comprises part of an environmental sample.

69. The method of claim 64, wherein the target protein comprises part of a biological sample.

70. The method of claim 64, further comprising the step of repeating steps (a)-(c).

71. The method of claim 64, wherein the target protein comprises a portion of a pathogen.

72. The method of claim 64, wherein the target protein is selected from the group consisting of Aflatoxins, Botulinum toxins, Clostridium toxins, Conotoxins, Ricins, Saxitoxins, Shiga toxins. Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins.

73. The method of claim 64, wherein the target protein comprises a portion of one or more bioterror agent.

74. The method of claim 64, wherein the pathogen sample comprises a bioterror agent selected from the group consisting of Bacillus, Yersinia, Francisella, Vibrio, Brucella, Clostridium.

75. The method of claim 64, wherein the pathogen sample comprises a bioterror agent selected from the group consisting of smallpox viruses, flavivirus, coronavirus, hemorrhagic fever viruses, and neuropathologic viruses.

76. A method for determining which thioaptamers bound to a target protein comprising the steps of:
(a) deproteinating with urea to remove bound protein one or more thio-aptamer beads that have bound specifically to the target protein,
(b) subjecting the deproteinated beads to PCR to produce a PCR product;
(c) sequencing the PCR product to obtain the sequence of the aptamer on the one or more beads that bound the target protein; and
(d) contacting a one bead-one aptamer library comprising all the aptamers the thio-aptamer beads of step (a) to identify one or more thio-aptamers that bind specifically to the target protein.

77. The method of claim 76, further comprising the step of repeating (a)-(d).

78. A method for isolating a pathogen comprising the steps of:
preparing a pathogen-specific aptamer array to screen one or more samples for the presence of a pathogen that binds specifically to the pathogen-specific aptamer array.

79. A pathogen-specific aptamer array comprising:
one or more isolated nucleic acid aptamers that bind specifically to a pathogen on an array, wherein a pathogen that binds to the isolated nucleic acid aptamers on the array is detected to identify the presence of the pathogen in a sample.

80. A pathogen-specific aptamer screening system comprising:
one or more beads comprising an immobilized pathogen-specific aptamer, wherein a pathogen that binds specifically to the immobilized pathogen-specific aptamer is detected upon binding to the immobilized pathogen-specific aptamer.

81. The system of claim 80, wherein the one or more beads are contacted with a sample suspected of comprising one or more pathogens.

82. The system of claim 80, wherein the one or more beads are contacted with a sample suspected of comprising one or more pathogens and the beads are sorted based on the pathogen binding to the aptamer.

83. The system of claim 80, wherein the one or more beads are contacted with a sample suspected of comprising one or more pathogens and sorted manually.

84. The system of claim 80, wherein the one or more beads are contacted with a sample suspected of comprising one or more pathogens and the one or more beads are sorted magnetically.

85. The system of claim 80, wherein the one or more beads are contacted with a sample suspected of comprising one or more pathogens and sorted using an automated bead sorting apparatus.

86. The system of claim 80, wherein the one or more beads are contacted with a sample suspected of comprising one or more pathogens and sorted using a flow cytometer.

87. The system of claim 80, further comprising contacting the sample suspected of comprising one or more pathogens labeled with a fluorescent label.

88. The system of claim 80, further comprising contacting the sample suspected of comprising one or more pathogens labeled with a radiolabel.

89. The system of claim 80, further comprising:
a labeled sample suspected of comprising one or more pathogens, wherein the pathogens are contacted with the beads; and
a bead isolation system for separating the beads that that have bound a pathogen from those that have not.

90. The system of claim 80, further comprising:
a labeled sample suspected of comprising one or more pathogens, wherein the pathogens are contacted with the beads;
a bead isolation system for separating the beads that that have bound a pathogen from those that have not; and
identification of the pathogen bound to a sorted bead by SELDI mass spectrometry analysis.

91. The system of claim 80, further comprising:
a labeled sample suspected of comprising one or more pathogens, wherein the pathogens are contacted with the beads;
a bead isolation system for separating the beads that that have bound a pathogen from those that have not; and
identification of the pathogen bound to a sorted bead by cloning and sequencing of the aptamer.

92. The system of claim 80, further comprising:
a labeled sample suspected of comprising one or more pathogens, wherein the pathogens are contacted with the beads;
a bead isolation system for separating the beads that that have bound a pathogen from those that have not;
deproteination of the pathogen on the one or more beads; and
identification of the pathogen bound by sequencing of the pathogen.

93. A method for concentrating a pathogen out of a sample, comprising:
immobilizing a pathogen-specific aptamer on a substrate; and
exposing a sample suspected of comprising the pathogen to the immobilized aptamers.

94. The method of claim 93, wherein the sample comprises an environmental sample.

95. The method of claim 93, wherein the sample comprises a biological sample.

96. The method of claim 93, wherein the sample is selected from the group consisting of a water, a soil, a waste and an air sample or mixtures thereof.

97. The method of claim 93, further comprising the step of identifying the pathogen by ELISA.

98. The method of claim 93, further comprising the step of identifying a protein of a pathogen by ELISA, wherein the protein bound the aptamer.

99. The method of claim 93, further comprising the step of determining the identity of the pathogen bound to the pathogen-specific aptamer by SELDI-mass spectrometry analysis.

100. The method of claim 93, further comprising the step of identifying the pathogen bound to the pathogen-specific aptamer by cloning and sequencing of the aptamer.

101. The method of claim 93, further comprising the steps of:
deproteinating the pathogen bound to the pathogen-specific aptamer; and
identifying the pathogen bound by cloning and sequencing of the pathogen.

102. The method of claim 93, further comprising the steps of:
flowing over the aptamers a sample suspected of comprising a pathogen.

103. The method of claim 93, wherein a pathogen in the sample is detected using surface plasmon resonance.

104. The method of claim 93, wherein a pathogen in the sample is detected using a capacitance coupled device.

105. The method of claim 93, wherein the substrate comprises a semiconductor.

106. The method of claim 93, wherein the substrate comprises a semiconductor array.

107. The method of claim 93, wherein the substrate comprises a multi-well plate.

108. The method of claim 93, wherein the aptamer is immobilized onto the substrate using a linker.

110. A method for identifying a pathogen comprising the steps of:
immobilizing a pathogen-specific aptamer on a substrate;
exposing a sample suspected of comprising at least a portion of the pathogen to the immobilized aptamers; and
identifying the aptamer that bound the portion of the pathogen, wherein the aptamer is specific to at least a portion of the pathogen.

111. A filter for concentrating a pathogen, comprising a pathogen-specific aptamer immobilized on a substrate, wherein a sample suspected of comprising the pathogen is exposed to the immobilized pathogen-specific aptamers to concentrate the pathogen.

112. The filter of claim 111, wherein the substrate comprises a paper filter.

113. The filter of claim 111, wherein the substrate comprises a silicon-based material.

114. The filter of claim 111, wherein the substrate comprises a capacitance-coupled device.

115. The filter of claim 111, wherein the substrate comprises a three-dimensional matrix.

116. The filter of claim 111, wherein the substrate comprises a hydrophobic material.

117. The filter of claim 111, wherein the substrate comprises a hydrophilic material.

118. A method of concentrating a pathogen comprising the step of:
immobilizing a pathogen-specific partially thio-modified aptamer on a substrate, wherein a sample suspected of comprising a pathogen is exposed to the immobilized pathogen-specific aptamers to concentrate the pathogen.

119. A cancer-specific aptamer screening system comprising:
one or more beads comprising an immobilized cancer-specific aptamer, wherein at least a portion of a cancer cell that binds specifically to the immobilized cancer-specific aptamer is detected.

120. The system of claim 119, wherein the one or more beads are contacted with a sample suspected of comprising one or more cancer cells.

121. The system of claim 119, wherein the one or more beads are contacted with a sample suspected of comprising one or more labeled cancer cell and the beads are sorted based on detection of the labeled cancer cell.

122. The system of claim 119, wherein the one or more beads are contacted with a sample suspected of comprising one or more cancer cells and the beads sorted based on cancer cell-aptamer binding.

123. The system of claim 119, wherein the one or more beads are contacted with a sample suspected of comprising one or more cancer cells and sorted using a bead sorting system.

124. The system of claim 119, wherein the one or more beads are contacted with a sample suspected of comprising one or more cancer cells and sorted using a flow cytometer.

125. The system of claim 119, further comprising contacting the sample suspected of comprising one or more cancer cells labeled with a fluorescent label.

126. The system of claim 119, further comprising contacting the sample suspected of comprising one or more cancer cells labeled with a radiolabel.

127. The system of claim 119, further comprising:
a labeled sample suspected of comprising one or more cancer cells, wherein the cancer cells are contacted with the beads; and
a bead isolation system for separating the beads that that have bound to a portion of cancer cell from those that have not.

128. The system of claim 119, further comprising:
a labeled sample suspected of comprising one or more cancer cells, wherein the cancer cells are contacted with the beads;
a bead isolation system for separating the beads that that have bound a portion of the cancer cells from those that have not; and
a determination of the identify of the portion of the cancer cells bound to a sorted bead by mass spectrometric (SELDI) analysis.

129. The system of claim 119, further comprising:
a labeled sample suspected of comprising one or more cancer cells, wherein the cancer cells are contacted with the beads;
a bead isolation system for separating the beads that that have bound a cancer cells from those that have not; and
identification of the cancer cells bound to a sorted bead by cloning and sequencing of the aptamer.

130. The system of claim 119, further comprising:
a labeled sample suspected of comprising one or more cancer cells, wherein the cancer cells are contacted with the beads;
a bead isolation system for separating the beads that that have bound a cancer cells from those that have not;
deproteination of the cancer cells on the one or more beads; and
identification of the aptamer bound by cloning and sequencing of the aptamer.

131. The system of claim 119, wherein a bead isolation system for separating the beads that that have bound a portion of the cancer cells from those that have not by magnetic separation.

132. A thioaptamer comprising:
a cancer-specific thioaptamer, wherein the aptamer binds to a specific cancer cell aptamer epitope.

133. The thioaptamer of claim 132, wherein the cancer-specific thioaptamer further comprises a label.

134. The thioaptamer of claim 132, wherein the cancer-specific thioaptamer further comprises a radiolabel.

135. The thioaptamer of claim 132, wherein the cancer-specific thioaptamer further comprises a fluorescent label.

136. The thioaptamer of claim 132, wherein the cancer-specific thioaptamer further comprises a toxin.

137. The thioaptamer of claim 132, wherein the cancer-specific thioaptamer further comprises an aptazyme.

138. The thioaptamer of claim 132, wherein the cancer-specific thioaptamer further comprises an energy-absorbing molecule.

139. The thioaptamer of claim 132, wherein the cancer-specific thioaptamer further comprises an ionophore.

140. The thioaptamer of claim 132, further comprising a potentiator selected from the group consisting of procodazole, triprolidine, propionic acid, monensin, an anti-sense inhibitor of the RAD51 gene, bromodeoxyuridine, dipyridamole, indomethacin, a monoclonal antibody, an anti-transferrin receptor immunotoxin, metoclopramide, 7-thia-8-oxoguanosine, N-solanesyl-N,N′-bis(3,4-dimethoxybenzyl)ethylenediamine, N-[4[(4-fluorphenyl)sulfonyl]phenyl]acetamide, leucovorin, heparin, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative having potentiator activity, dimethyl sulfoxide and mixtures thereof.

141. The thioaptamer of claim 132, wherein the cancer-specific thioaptamer further comprises a liposome.

142. The thioaptamer of claim 132, wherein the cancer-specific thioaptamer further comprises a pharmaceutically acceptable salt.

143. The thioaptamer of claim 132, wherein the cancer-specific thioaptamer is lyophilized.

144. The thioaptamer of claim 132, wherein the cancer-specific thioaptamer is in injectable form.

145. The thioaptamer of claim 132, wherein the cancer-specific thioaptamer comprises one or more thio-modified nucleotides.

146. The thioaptamer of claim 132, wherein the cancer-specific thioaptamer comprises one or more partially dithio-modified nucleotides.

147. A pharmaceutical composition comprising a therapeutically effective amount of a thioaptamer specific for a virally-infected cell having SEQ ID NOS.: 30 to 107, or mixtures thereof.

148. The composition of claim 139, wherein the aptamer is specific for a flavivirus-infected cell.

149. The composition of claim 139, wherein the aptamer comprises the sequence 5′-GGC CTG TAC ACG TGT ACA CC-3′ (SEQ ID NO.: 1).

150. A method of treatment with a thioaptamer comprising:
identifying a patient in need of thioaptamer therapy and providing a therapeutically effective amount of the thioaptamer to the patient.

151. The method of claim 150, wherein the patient is infected with a pathogen.

152. A method of treating a target cell with a thioaptamer comprising:
contacting a target cell with a thioaptamer, wherein the thioaptamer binds specifically to a thioaptamer target within the target cell to modify the physiology of the target cell.

153. The method of claim 152, wherein the thioaptamer further comprises an agent that leads to the death of the target cell.

154. The method of claim 152, wherein the target cell comprises a cancer cell.

155. The method of claim 153, wherein the agent is selected from the group consisting of Aflatoxins, Botulinum toxins, Clostridium toxins, Conotoxins, Ricins, Saxitoxins, Shiga toxins, Staphylococcus aureus toxins, Tetrodotoxins, Verotoxins, Microcystins (Cyanginosin), Abrins, Cholera toxins, Tetanus toxins, Trichothecene mycotoxins, Modeccins, Volkensins, Viscum Album Lectin 1, Streptococcal toxins, Pseudomonas A toxins, Diphtheria toxins, Listeria monocytogenes toxins, Bacillus anthracis toxic complexes, Francisella tularensis toxins, whooping cough pertussis toxins, Yersinia pestis toxic complexes, Yersinia enterocolytica enterotoxins, Brucella toxins, and Pasteurella toxins, a radiologic agent, or active subunits thereof.

156. The method of claim 152, further comprising the step of adding a potentiator selected from the group consisting of procodazole, triprolidine, propionic acid, monensin, an anti-sense inhibitor of the RAD51 gene, bromodeoxyuridine, dipyridamole, indomethacin, a monoclonal antibody, an anti-transferrin receptor immunotoxin, metoclopramide, 7-thia-8-oxoguanosine, N-solanesyl-N,N′-bis(3,4-dimethoxybenzyl)ethylenediamine, N-[4[(4-fluorphenyl)sulfonyl]phenyl]acetamide, leucovorin, heparin, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative having potentiator activity, dimethyl sulfoxide and mixtures thereof

157. The method of claim 152, wherein the target cell comprises a prostate, breast, colon, lung, pancreatic, throat, liver, ovarian, skin or lymphoid cancer cell.

158. A kit for detecting a pathogen comprising:
a pathogen-specific aptamer; and
instructions for use of the pathogen-specific aptamer to detect a binding target.

159. The kit of claim 158, wherein the binding target is a protein, a glycoprotein, a lipid, a carbohydrate, a nucleic acid or combinations thereof.

160. The kit of claim 158, wherein the binding target comprises a portion of a pathogen.

161. The kit of claim 158, wherein the binding target comprises a portion of a virus, a prokaryote or a eukaryote.

162. The kit of claim 158, wherein the aptamer comprises a thioaptamer.

163. The kit of claim 158, wherein the aptamer comprises a concatenated aptamer.

164. The kit of claim 158, wherein the aptamer is immobilized on a substrate.

165. The kit of claim 158, wherein the kit further comprises a pathogen-specific antibody comprising a detectable label.

166. The kit of claim 158, wherein the kit further comprises a second pathogen-specific agent comprising a detectable label.

167. A kit for concentrating a pathogen from a sample comprising:
one or more immobilized pathogen-specific thioaptamers; and
instructions for use of the one or more immobilized pathogen-specific thioaptamers.

168. A kit for concentrating a pathogen in a sample comprising:
an immobilized pathogen-specific aptamer on a substrate; and
instructions for use of the immobilized pathogen-specific aptamer to concentrate the pathogen.

169. The kit of claim 168, wherein the pathogen-specific aptamer binds to a protein, a glycoprotein, a lipid, a carbohydrate, a nucleic acid or combinations thereof of the pathogen.

170. The kit of claim 168, wherein the pathogen comprises a portion of a virus, a prokaryote or a eukaryote.

171. The kit of claim 168, wherein the aptamer comprises a thioaptamer.

172. The kit of claim 168, wherein the aptamer comprises a concatenated aptamer.

173. The kit of claim 168, wherein the substrate comprises a paper, a hydrophobic filter, a hydrophilic filter, a semiconductor, a metal, a bead, a glass, a plastic plate or a multi-well plate.

174. A kit for identifying a target-specific aptamer comprising
a thioaptamer bead library; and
instructions for isolating one or more of the thioaptamer beads from the thioaptamer bead library after contacting the thioaptamer beads to an thioaptamer binding target.

175. A kit for treating a pathogenic infection comprising:
a vial comprising one or more pathogen-specific thioaptamers in a pharmaceutically acceptable form; and
instructions for use of the pathogen-specific thioaptamers to treat infection with a pathogen.

176. A kit for treating a cancer comprising:
a vial comprising one or more cancer cell-specific thioaptamers in a pharmaceutically acceptable form; and
instructions for use of the cancer-specific thioaptamers to treat a cancer.

177. A kit for cosmetic treatment comprising:
a vial comprising one or more cell-specific thioaptamers attached to an agent that causes cell death in a pharmaceutically acceptable form; and
instructions for use of the cell-specific thioaptamers to kill an unwanted cell.

178. A cosmetic composition comprising, in a cosmetically acceptable aqueous medium at least one target-cell specific thioaptamer, wherein the thioaptamer further comprises an agent that leads to cell death.

179. A method for cosmetic treatment for eliminating unwanted cell comprising the administration of a composition comprising at least an effective amount of a thioaptamer conjugated to an agent that causes cell death, or a salt thereof.


SEQUENCE LISTING (142 sequences)

SEQ ID NO: 1 - DNA, 20 bp, Artificial Sequence
  Description of Artificial Sequence Synthetic oligonucleotide
ggcctgtacacgtgtacacc

SEQ ID NO: 2 - DNA, 14 bp, Homo sapiens
ctgttcgggcgcca

SEQ ID NO: 3 - DNA, 10 bp, Unknown Sequence
  Description of Unknown Sequence Illustrative NF-kB consensus-like sequence
ggggacttcc

SEQ ID NO: 4 - DNA, 42 bp, Unknown Organism
  Description of Unknown Organism Illustrative NF-kB consensus-like sequence
ccaggagattccacccaggagattccacccaggagattccac

SEQ ID NO: 5 - DNA, 22 bp, Artificial Sequence
  Description of Artificial Sequence Synthetic oligonucleotide
ctgtgttcttgtgccgtgtccc

SEQ ID NO: 6 - DNA, 22 bp, Artificial Sequence
  Description of Artificial Sequence Synthetic oligonucleotide
ctgtgttcttgtgtcgtgtccc

SEQ ID NO: 7 - DNA, 22 bp, Artificial Sequence
  Description of Artificial Sequence Synthetic oligonucleotide
ctgtgttcttgtgtcgtgcccc

SEQ ID NO: 8 - DNA, 22 bp, Artificial Sequence
  Description of Artificial Sequence Synthetic oligonucleotide
ccgtgttcttgtgccgtgtccc

SEQ ID NO: 9 - DNA, 22 bp, Artificial Sequence
  Description of Artificial Sequence Synthetic oligonucleotide
ccgtgttcttgtgtcgtgtccc

SEQ ID NO: 10 - DNA, 23 bp, Artificial Sequence
  Description of Artificial Sequence Synthetic oligonucleotide
cggggtgttgtcctgtgctctcc

SEQ ID NO: 11 - DNA, 23 bp, Artificial Sequence
  Description of Artificial Sequence Synthetic oligonucleotide
cggggtgttctcctgtgctctcc

SEQ ID NO: 12 - DNA, 23 bp, Artificial Sequence
  Description of Artificial Sequence Synthetic oligonucleotide
cggggtggtgtggcgaggcggcc

SEQ ID NO: 13 - DNA, 23 bp, Artificial Sequence
  Description of Artificial Sequence Synthetic oligonucleotide
cggggtggtgcggcgaggcggcc

SEQ ID NO: 14 - DNA, 23 bp, Artificial Sequence
  Description of Artificial Sequence Synthetic oligonucleotide
cggggtgtgctgctgcgggcggc

SEQ ID NO: 15 - DNA, 23 bp, Artificial Sequence
  Description of Artificial Sequence Synthetic oligonucleotide
cggggtgtgctgctgcgggcggc

SEQ ID NO: 16 - DNA, 22 bp, Artificial Sequence
  Description of Artificial Sequence Synthetic oligonucleotide
ctgtgytcttgtgtygtgtccc

SEQ ID NO: 17 - DNA, 22 bp, Artificial Sequence
  Description of Artificial Sequence Synthetic oligonucleotide
ctgtgagtcgactgatgacggt

SEQ ID NO: 18 - DNA, 15 bp, Artificial Sequence
  Description of Artificial Sequence Synthetic oligonucleotide primer
ggatccggtggtctg

SEQ ID NO: 19 - DNA, 15 bp, Artificial Sequence
  Description of Artificial Sequence Synthetic oligonucleotide primer
cctactcgcgaattc

SEQ ID NO: 20 - RNA, 31 nt, Artificial Sequence
  Description of Artificial Sequence Synthetic oligonucleotide
gauccugaaacuguuuuaagguuggccgauc

SEQ ID NO: 21 - RNA, 31 nt, Artificial Sequence
  Description of Artificial Sequence Synthetic oligonucleotide
cuaggacuuggcacaaccgucacacugcuau

SEQ ID NO: 22 - DNA, 21 bp, Artificial Sequence
  Description of Artificial Sequence Synthetic oligonucleotide
tgtgcagggactgatgacggt

SEQ ID NO: 23 - DNA, 22 bp, Artificial Sequence
  Description of Artificial Sequence Synthetic oligonucleotide
atgcacatctcaggatgacggt

SEQ ID NO: 24 - DNA, 22 bp, Artificial Sequence
  Description of Artificial Sequence Synthetic oligonucleotide
agttgcaggtcaggacccattt

SEQ ID NO: 25 - DNA, 14 bp, Artificial Sequence
  Description of Artificial Sequence Synthetic oligonucleotide
ccaggagattccac

SEQ ID NO: 26 - DNA, 14 bp, Artificial Sequence
  Description of Artificial Sequence Synthetic oligonucleotide
ccagtgactcagtg

SEQ ID NO: 27 - DNA, 14 bp, Artificial Sequence
  Description of Artificial Sequence Synthetic oligonucleotide
ttgcgcgcaacatg

SEQ ID NO: 28 - DNA, 14 bp, Artificial Sequence
  Description of Artificial Sequence Synthetic oligonucleotide
ccagtgactcagtg

SEQ ID NO: 29 - DNA, 14 bp, Artificial Sequence
  Description of Artificial Sequence Synthetic oligonucleotide
ttgcgcgcaacatg

SEQ ID NO: 30 - DNA, 30 bp, Artificial Sequence
  Description of Artificial Sequence Synthetic oligonucleotide
gagctgagtcccaccttttctgcgccgctc

SEQ ID NO: 31 - DNA, 30 bp, Artificial Sequence
  Description of Artificial Sequence Synthetic oligonucleotide
aacgaatcgtttgtgaacgacaaacttttc

SEQ ID NO: 32 - DNA, 30 bp, Artificial Sequence
  Description of Artificial Sequence Synthetic oligonucleotide
tgcgaccacacctgtagtcatggcttaaat

SEQ ID NO: 33 - DNA, 30 bp, Artificial Sequence
  Description of Artificial Sequence Synthetic oligonucleotide
ctatatcagctatcaacaggaaaaactgag

SEQ ID NO: 34 - DNA, 30 bp, Artificial Sequence
  Description of Artificial Sequence Synthetic oligonucleotide
gctgtatacccatccatgcgatgcccctac

SEQ ID NO: 35 - DNA, 30 bp, Artificial Sequence
  Description of Artificial Sequence Synthetic oligonucleotide
gcctgtcgtgtgtatatgcacattcacctg

SEQ ID NO: 36 - DNA, 30 bp, Artificial Sequence
  Description of Artificial Sequence Synthetic oligonucleotide
cgcccaaacttgcacagctacacgaatagc

SEQ ID NO: 37 - DNA, 30 bp, Artificial Sequence
  Description of Artificial Sequence Synthetic oligonucleotide
agttggccacagcaaaaactnccctcgcgg

SEQ ID NO: 38 - DNA, 30 bp, Artificial Sequence
  Description of Artificial Sequence Synthetic oligonucleotide
ggcggttctgtccatgcccatactccccac

SEQ ID NO: 39 - DNA, 30 bp, Artificial Sequence
  Description of Artificial Sequence Synthetic oligonucleotide
cctccttccacctctaggcggatagagggt

SEQ ID NO: 40 - DNA, 29 bp, Artificial Sequence
  Description of Artificial Sequence Synthetic oligonucleotide
gtctgaatttacgccttctatccgctccc

SEQ ID NO: 41 - DNA, 30 bp, Artificial Sequence
  Description of Artificial Sequence Synthetic oligonucleotide
atgttgcagctccgattcaatgccgcacac

SEQ ID NO: 42 - DNA, 30 bp, Artificial Sequence
  Description of Artificial Sequence Synthetic oligonucleotide
ggcctgtatacacgtgtacacccacacagc

SEQ ID NO: 43 - DNA, 12 bp, Artificial Sequence
  Description of Artificial Sequence Synthetic oligonucleotide
ggcgtgtacacc

SEQ ID NO: 44 - DNA, 11 bp, Artificial Sequence
  Description of Artificial Sequence Synthetic oligonucleotide
gcctgtacacc

SEQ ID NO: 45 - DNA, 20 bp, Artificial Sequence
  Description of Artificial Sequence Synthetic oligonucleotide
ggcctgtacacgtgtacacc

SEQ ID NO: 46 - DNA, 12 bp, Artificial Sequence
  Description of Artificial Sequence Synthetic oligonucleotide
ggcctgtacacg

SEQ ID NO: 47 - DNA, 20 bp, Artificial Sequence
  Description of Artificial Sequence Synthetic oligonucleotide
ggcctgtacacgtgtacacc

SEQ ID NO: 48 - DNA, 20 bp, Artificial Sequence
  Description of Artificial Sequence Synthetic oligonucleotide
ggcggctgtacatgtacacc

SEQ ID NO: 49 - DNA, 12 bp, Artificial Sequence
  Description of Artificial Sequence Synthetic oligonucleotide
ggcctgtacacc

SEQ ID NO: 50 - DNA, 20 bp, Artificial Sequence
  Description of Artificial Sequence Synthetic oligonucleotide
ggcccgtacacgtgtacacc

SEQ ID NO: 51 - DNA, 20 bp, Artificial Sequence
  Description of Artificial Sequence Synthetic oligonucleotide
ggcctgtacacgtgtacacc

SEQ ID NO: 52 - DNA, 20 bp, Artificial Sequence
  Description of Artificial Sequence Synthetic oligonucleotide
ggcccgtacacgtgtacacc

SEQ ID NO: 53 - DNA, 30 bp, Artificial Sequence
  Description of Artificial Sequence Synthetic oligonucleotide
accgtgcagatggagtgggaatccagtacc

SEQ ID NO: 54 - DNA, 30 bp, Artificial Sequence
  Description of Artificial Sequence Synthetic oligonucleotide
ttgttgtccataacgtcgcaacacaagcac

SEQ ID NO: 55 - DNA, 30 bp, Artificial Sequence
  Description of Artificial Sequence Synthetic oligonucleotide
cggttggggagcgcaatcaagatgggacgc

SEQ ID NO: 56 - DNA, 30 bp, Artificial Sequence
  Description of Artificial Sequence Synthetic oligonucleotide
gcgtgcatctacacatacctgcactatgca

SEQ ID NO: 57 - DNA, 33 bp, Artificial Sequence
  Description of Artificial Sequence Synthetic oligonucleotide
ggaatgcgngngngnacnggatnntnccaccan

SEQ ID NO: 58 - DNA, 31 bp, Artificial Sequence
  Description of Artificial Sequence Synthetic oligonucleotide
tgcgcncgnggaccccctcccaancttcant

SEQ ID NO: 59 - DNA, 30 bp, Artificial Sequence
  Description of Artificial Sequence Synthetic oligonucleotide
nnncgnncntggaggggtnaagngngacnc

SEQ ID NO: 60 - DNA, 30 bp, Artificial Sequence
  Description of Artificial Sequence Synthetic oligonucleotide
gtggttgaatctacgcgacaactctgtccc

SEQ ID NO: 61 - DNA, 30 bp, Artificial Sequence
  Description of Artificial Sequence Synthetic oligonucleotide
gtgaacacaagttggatgtagggcgccagg

SEQ ID NO: 62 - DNA, 30 bp, Artificial Sequence
  Description of Artificial Sequence Synthetic oligonucleotide
tccagcgcaatacctacctaggcagatcac

SEQ ID NO: 63 - DNA, 30 bp, Artificial Sequence
  Description of Artificial Sequence Synthetic oligonucleotide
tgcccacatgcagtagtacaatcccgaccc

SEQ ID NO: 64 - DNA, 30 bp, Artificial Sequence
  Description of Artificial Sequence Synthetic oligonucleotide
tccgcaattcagcattcccatgcatacagg

SEQ ID NO: 65 - DNA, 30 bp, Artificial Sequence
  Description of Artificial Sequence Synthetic oligonucleotide
ctgtacgcccagagcagacattacccgcac

SEQ ID NO: 66 - DNA, 30 bp, Artificial Sequence
  Description of Artificial Sequence Synthetic oligonucleotide
gcgtgtacgttcaacccaagcactggacac

SEQ ID NO: 67 - DNA, 30 bp, Artificial Sequence
  Description of Artificial Sequence Synthetic oligonucleotide
gccttccacaacagcggaatgcacaagcac

SEQ ID NO: 68 - DNA, 30 bp, Artificial Sequence
  Description of Artificial Sequence Synthetic oligonucleotide
ggttgacattgctgttccatccagtgaacg

SEQ ID NO: 69 - DNA, 30 bp, Artificial Sequence
  Description of Artificial Sequence Synthetic oligonucleotide
cccaccgacgtttccttggtgtaccagcac

SEQ ID NO: 70 - DNA, 30 bp, Artificial Sequence
  Description of Artificial Sequence Synthetic oligonucleotide
tggtntcccnaccactgnacaccttgaagc

SEQ ID NO: 71 - DNA, 30 bp, Artificial Sequence
  Description of Artificial Sequence Synthetic oligonucleotide
ggggcgcgcgttgacactgtacactgcacc

SEQ ID NO: 72 - DNA, 30 bp, Artificial Sequence
  Description of Artificial Sequence Synthetic oligonucleotide
ccgcgtcatatagcctcatcaagtggaccg

SEQ ID NO: 73 - DNA, 30 bp, Artificial Sequence
  Description of Artificial Sequence Synthetic oligonucleotide
gtggtagaccacactagaccacatcctaac

SEQ ID NO: 74 - DNA, 30 bp, Artificial Sequence
  Description of Artificial Sequence Synthetic oligonucleotide
gcctgcagctccagtgtgcacactcaccac

SEQ ID NO: 75 - DNA, 30 bp, Artificial Sequence
  Description of Artificial Sequence Synthetic oligonucleotide
nnnngcagcannncaatncannnccccccn

SEQ ID NO: 76 - DNA, 13 bp, Artificial Sequence
  Description of Artificial Sequence Synthetic oligonucleotide
tccagtctgcacc

SEQ ID NO: 77 - DNA, 30 bp, Artificial Sequence
  Description of Artificial Sequence Synthetic oligonucleotide
gtgnanncntggtgncanctccaacacnnn

SEQ ID NO: 78 - DNA, 14 bp, Artificial Sequence
  Description of Artificial Sequence Synthetic oligonucleotide
gctgtccacaccac

SEQ ID NO: 79 - DNA, 23 bp, Artificial Sequence
  Description of Artificial Sequence Synthetic oligonucleotide
gtgtgcctacnctgcaacacgtg

SEQ ID NO: 80 - DNA, 27 bp, Artificial Sequence
  Description of Artificial Sequence Synthetic oligonucleotide
gcgtgcacactacaagtccacacgtca

SEQ ID NO: 81 - DNA, 29 bp, Artificial Sequence
  Description of Artificial Sequence Synthetic oligonucleotide
gctgtcctgtgtacacggtgcaatgcaac

SEQ ID NO: 82 - DNA, 30 bp, Artificial Sequence
  Description of Artificial Sequence Synthetic oligonucleotide
gccgtccatacagtttaacgcgaacccagc

SEQ ID NO: 83 - DNA, 30 bp, Artificial Sequence
  Description of Artificial Sequence Synthetic oligonucleotide
tcgtgtccatgcccgaacagaccacaacag

SEQ ID NO: 84 - DNA, 30 bp, Artificial Sequence
  Description of Artificial Sequence Synthetic oligonucleotide
tgctgccaacacaaccagactccccaggcg

SEQ ID NO: 85 - DNA, 30 bp, Artificial Sequence
  Description of Artificial Sequence Synthetic oligonucleotide
gcgtgttgttgtacagtccccagttgtacc

SEQ ID NO: 86 - DNA, 19 bp, Artificial Sequence
  Description of Artificial Sequence Synthetic oligonucleotide
gcgtgtgcacagtccaacc

SEQ ID NO: 87 - DNA, 30 bp, Artificial Sequence
  Description of Artificial Sequence Synthetic oligonucleotide
gcgctgtatatccatgtgtggtcctaacgc

SEQ ID NO: 88 - DNA, 20 bp, Artificial Sequence
  Description of Artificial Sequence Synthetic oligonucleotide
gtgtatcgtgtggtgcagcc

SEQ ID NO: 89 - DNA, 29 bp, Artificial Sequence
  Description of Artificial Sequence Synthetic oligonucleotide
tgcatccataccacgatgtacnnaacaac

SEQ ID NO: 90 - DNA, 30 bp, Artificial Sequence
  Description of Artificial Sequence Synthetic oligonucleotide
tgctccaacgtctccatccaacgcgagagg

SEQ ID NO: 91 - DNA, 30 bp, Artificial Sequence
  Description of Artificial Sequence Synthetic oligonucleotide
gcgtgccgacattcacagtgttcanggaac

SEQ ID NO: 92 - DNA, 28 bp, Artificial Sequence
  Description of Artificial Sequence Synthetic oligonucleotide
gctgtacacctggactggacaccagcac

SEQ ID NO: 93 - DNA, 19 bp, Artificial Sequence
  Description of Artificial Sequence Synthetic oligonucleotide
gcgtgtgcacagtccaaca

SEQ ID NO: 94 - DNA, 16 bp, Artificial Sequence
  Description of Artificial Sequence Synthetic oligonucleotide
gcctccacacgcacag

SEQ ID NO: 95 - DNA, 26 bp, Artificial Sequence
  Description of Artificial Sequence Synthetic oligonucleotide
gcgtgtccacgtgcacaccccaccgc

SEQ ID NO: 96 - DNA, 19 bp, Artificial Sequence
  Description of Artificial Sequence Synthetic oligonucleotide
gcgtgtgcacagtccaacc

SEQ ID NO: 97 - DNA, 24 bp, Artificial Sequence
  Description of Artificial Sequence Synthetic oligonucleotide
gcctgcacacctgtacaccgcggc

SEQ ID NO: 98 - DNA, 30 bp, Artificial Sequence
  Description of Artificial Sequence Synthetic oligonucleotide
gcgtgtgcacactgtactgtacaccccagg

SEQ ID NO: 99 - DNA, 12 bp, Artificial Sequence
  Description of Artificial Sequence Synthetic oligonucleotide
tctatccacggc

SEQ ID NO: 100 - DNA, 19 bp, Artificial Sequence
  Description of Artificial Sequence Synthetic oligonucleotide
gcctgtatacactcccagc

SEQ ID NO: 101 - DNA, 19 bp, Artificial Sequence
  Description of Artificial Sequence Synthetic oligonucleotide
gcgtgtgcacagtccaacc

SEQ ID NO: 102 - DNA, 30 bp, Artificial Sequence
  Description of Artificial Sequence Synthetic oligonucleotide
tggtgccctactgcatgccgcccccacacg

SEQ ID NO: 103 - DNA, 19 bp, Artificial Sequence
  Description of Artificial Sequence Synthetic oligonucleotide
cgctgtgcacagtccaacc

SEQ ID NO: 104 - DNA, 19 bp, Artificial Sequence
  Description of Artificial Sequence Synthetic oligonucleotide
gcgtgtgcacagtccccac

SEQ ID NO: 105 - DNA, 30 bp, Artificial Sequence
  Description of Artificial Sequence Synthetic oligonucleotide
gcgttgcatgtgancncccagtaccacagg

SEQ ID NO: 106 - DNA, 30 bp, Artificial Sequence
  Description of Artificial Sequence Synthetic oligonucleotide
gcgtgcacttggacacctccacatcgaccc

SEQ ID NO: 107 - DNA, 30 bp, Artificial Sequence
  Description of Artificial Sequence Synthetic oligonucleotide
nttgtgctggngcagngnacatgnacacgc

SEQ ID NO: 108 - DNA, 15 bp, Artificial Sequence
  Description of Artificial Sequence Synthetic oligonucleotide primer
ggatccggtggtctg

SEQ ID NO: 109 - DNA, 15 bp, Artificial Sequence
  Description of Artificial Sequence Synthetic oligonucleotide primer
cctactcgcgaattc

SEQ ID NO: 110 - DNA, 14 bp, Artificial Sequence
  Description of Artificial Sequence Synthetic oligonucleotide
atgngaatttncca

SEQ ID NO: 111 - DNA, 14 bp, Artificial Sequence
  Description of Artificial Sequence Synthetic oligonucleotide
ggagngcncaggac

SEQ ID NO: 112 - DNA, 14 bp, Artificial Sequence
  Description of Artificial Sequence Synthetic oligonucleotide
ggaggactttccac

SEQ ID NO: 113 - DNA, 14 bp, Artificial Sequence
  Description of Artificial Sequence Synthetic oligonucleotide
ggaggacattgcac

SEQ ID NO: 114 - DNA, 14 bp, Artificial Sequence
  Description of Artificial Sequence Synthetic oligonucleotide
ggaggaccttccac

SEQ ID NO: 115 - DNA, 14 bp, Artificial Sequence
  Description of Artificial Sequence Synthetic oligonucleotide
ggaggaccttgcac

SEQ ID NO: 116 - DNA, 14 bp, Artificial Sequence
  Description of Artificial Sequence Synthetic oligonucleotide
ggaggacntttcac

SEQ ID NO: 117 - DNA, 14 bp, Artificial Sequence
  Description of Artificial Sequence Synthetic oligonucleotide
ggaggacctttcac

SEQ ID NO: 118 - DNA, 14 bp, Artificial Sequence
  Description of Artificial Sequence Synthetic oligonucleotide
gggatggtcaggac

SEQ ID NO: 119 - DNA, 14 bp, Artificial Sequence
  Description of Artificial Sequence Synthetic oligonucleotide
gggcggatcaggac

SEQ ID NO: 120 - DNA, 14 bp, Artificial Sequence
  Description of Artificial Sequence Synthetic oligonucleotide
gggaagatcaggac

SEQ ID NO: 121 - DNA, 14 bp, Artificial Sequence
  Description of Artificial Sequence Synthetic oligonucleotide
ggggtgatcaggac

SEQ ID NO: 122 - DNA, 14 bp, Artificial Sequence
  Description of Artificial Sequence Synthetic oligonucleotide
ggagtgctcaggca

SEQ ID NO: 123 - DNA, 14 bp, Artificial Sequence
  Description of Artificial Sequence Synthetic oligonucleotide
ggagcggtgtccac

SEQ ID NO: 124 - DNA, 14 bp, Artificial Sequence
  Description of Artificial Sequence Synthetic oligonucleotide
gggagggattacca

SEQ ID NO: 125 - DNA, 14 bp, Artificial Sequence
  Description of Artificial Sequence Synthetic oligonucleotide
ggagcggtttgcca

SEQ ID NO: 126 - DNA, 14 bp, Artificial Sequence
  Description of Artificial Sequence Synthetic oligonucleotide
ggagcgatttccca

SEQ ID NO: 127 - DNA, 14 bp, Artificial Sequence
  Description of Artificial Sequence Synthetic oligonucleotide
ggagaggttttcca

SEQ ID NO: 128 - DNA, 14 bp, Artificial Sequence
  Description of Artificial Sequence Synthetic oligonucleotide
atagggcacaggac

SEQ ID NO: 129 - DNA, 14 bp, Artificial Sequence
  Description of Artificial Sequence Synthetic oligonucleotide
atagngcccaggac

SEQ ID NO: 130 - DNA, 14 bp, Artificial Sequence
  Description of Artificial Sequence Synthetic oligonucleotide
atagggcgcaggac

SEQ ID NO: 131 - DNA, 14 bp, Artificial Sequence
  Description of Artificial Sequence Synthetic oligonucleotide
ggagggcccagcac

SEQ ID NO: 132 - DNA, 14 bp, Artificial Sequence
  Description of Artificial Sequence Synthetic oligonucleotide
ggagagcacatcac

SEQ ID NO: 133 - DNA, 14 bp, Artificial Sequence
  Description of Artificial Sequence Synthetic oligonucleotide
ggagcgcgcaccac

SEQ ID NO: 134 - DNA, 14 bp, Artificial Sequence
  Description of Artificial Sequence Synthetic oligonucleotide
ggagcgcgcagcac

SEQ ID NO: 135 - DNA, 14 bp, Artificial Sequence
  Description of Artificial Sequence Synthetic oligonucleotide
ggagggctcagcac

SEQ ID NO: 136 - DNA, 14 bp, Artificial Sequence
  Description of Artificial Sequence Synthetic oligonucleotide
ggagagcacaacac

SEQ ID NO: 137 - DNA, 14 bp, Artificial Sequence
  Description of Artificial Sequence Synthetic oligonucleotide
ggagcgcgcatcac

SEQ ID NO: 138 - DNA, 14 bp, Artificial Sequence
  Description of Artificial Sequence Synthetic oligonucleotide
ggagagcgcaccac

SEQ ID NO: 139 - DNA, 30 bp, Artificial Sequence
  Description of Artificial Sequence Synthetic oligonucleotide
ccaggagattccacggatccggtggtctgt

SEQ ID NO: 140 - DNA, 45 bp, Artificial Sequence
  Description of Artificial Sequence Synthetic oligonucleotide
cctactcgcgaattcccaggagattccacggatccggtggtctgt

SEQ ID NO: 141 - DNA, 14 bp, Artificial Sequence
  Description of Artificial Sequence Synthetic poly-C oligonucleotide
cccccccccccccc

SEQ ID NO: 142 - DNA, 20 bp, Artificial Sequence
  Description of Artificial Sequence Synthetic oligonucleotide
ggcctgtacacgtgtacacc